(19) United States
(12) Patent Application Publication
(10) Pub. No.: US 2008/0306244 A1
(43) Pub. Date: Dec. 11, 2008

(54) Renta: an HIV immunogen and uses thereof

(76) Inventors: Tomas Hanke, Oxford (GB); Andrew James McMichael, Beckley (GB)

Correspondence Address: FROMMER LAWRENCE & HAUG, 745 FIFTH AVENUE- 10TH FL., NEW YORK, NY, 10151, US

(21) Appl. No.: 11/436,958

(22) Filed: May 12, 2006

Related U.S. Application Data
(63) Continuation-in-part of application No. PCT/US04/37699, filed on Nov. 12, 2004.
(60) Provisional application No. 60/519,420, filed on Nov. 12, 2003.

(51) Int. Cl. C07K 14/00

(52) U.S. Cl. 530/350

(57) ABSTRACT

The present invention provides artificial fusion proteins (AFPs) designed to elicit an anti-HIV immune response, as well as nucleic acid molecules and expression vectors encoding those proteins. The AFPs of the invention may comprise domains from various HIV proteins, including Reverse Trancriptase (RT), Env (gp41), Nef and Tat proteins, as well as at least one HIV CTL epitope associated with long-term, non-progression to AIDS; these domains are biologically-inactivated for one or more of the normal activity of those proteins or are partial protein sequences (and similarly biologically-inactivated). RENTA is an AFP in which the HIV domains are from an HIV Clade A consensus sequence and contains additional domains, useful for example, in monitoring expression levels or laboratory animal immune responses. Such domains are optionally included in the AFPs. Other aspects of the invention may include compositions for and methods of inducing an anti-HIV immune response in a subject, preferably using a DNA prime-MVA boost strategy, and preferably to induce a cell-mediated immune response.

### CROSS-REFERENCE TO RELATED APPLICATIONS

This application is a continuation-in-part application of international patent application Serial No. PCT/US2004/037699 filed Nov. 12, 2004, which claims priority from U.S. Provisional Patent Application Ser. No. 60/519,420, filed on Nov. 12, 2003.

Each of these applications and each of the documents cited in each of these applications (“application cited documents”), and each document referenced or cited in the application cited documents, either in the text or during the prosecution of those applications, as well as all arguments in support of patentability advanced during such prosecution, are hereby incorporated herein by reference. Various documents are also cited in this text (“application cited documents”). Each of the application cited documents, and each document cited or referenced in the application cited documents, is hereby incorporated herein by reference.

### FIELD OF THE INVENTION

This invention relates to artificial fusion proteins (AFPs) designed to elicit an anti-HIV immune response in a subject as well as nucleic acid molecules and expression vectors encoding those proteins. The AFPs, as well as nucleic acids and expression vectors encoding these proteins, can be administered alone or in combination to a subject to generate an anti-HIV immune response. The AFPs of the invention comprise domains from various HIV proteins, including Reverse Trancriptase (RT), Env (gp41), Nef and Tat proteins as well as at least one human HIV CTL epitope associated with long-term, non-progression to AIDS. The HIV proteins that form the domains are biologically inactivated for one or more of the normal activity of those proteins or are partial protein sequences (and similarly biologically-inactivated). RENTA is an AFP in which the HIV domains are from an HIV Clade A consensus sequence. RENTA also contains additional domains useful, for example, in monitoring protein expression levels or laboratory animal immune responses. Such domains are optionally included in the AFPs. Other aspects of the invention include compositions for and methods of inducing an anti-HIV immune response in a subject, preferably using a DNA prime-MVA boost strategy and preferably to induce a cell-mediated immune response.

### BACKGROUND OF THE INVENTION

As the world enters the third decade of the acquired immunodeficiency syndrome (AIDS) pandemic, evidence of its devastating impact is undeniable. In December 2002, 42 million people worldwide were living with HIV/AIDS and new infections were occurring at a rate of roughly 16,000 new infections daily. Five million people were newly infected in 2002 and 3.1 million people succumbed to AIDS in 2002. Of the 42 million people infected worldwide, 29.4 million live in sub-Saharan Africa, while 6 million live in south and southeast Asia (AIDS epidemic update, December 2002). Countries in these regions cannot afford the drugs that are currently used to treat infected people, and even if the drug prices were reduced, the costs associated with their clinical use are prohibitive. The consequences are a drastic lowering of life expectancy and enormous human social and economic problems. Thus, the development of a prophylactic vaccine that is cheaply and readily available is an urgent necessity.

AIDS is caused by human immunodeficiency virus (HIV) and is characterized by several clinical features including wasting syndromes, central nervous system degeneration and profound immunosuppression that results in opportunistic infections and malignancies. HIV is a member of the lentivirus family of animal retroviruses, which include the visna virus of sheep and the bovine, feline, and simian immunodeficiency viruses (SIV). Two closely related types of HIV, designated HIV-1 and HIV-2, have been identified thus far, of which HIV-1 is by far the most common cause of AIDS. However, HIV-2, which differs in genomic structure and antigenicity, causes a similar clinical syndrome.

The different isolates of HIV-1 have been classified into three groups: M (main), O (outlier) and N (non-M, non-O). The HIV-1 M group dominates the global HIV pandemic (Gaschen et al., (2002) Science 296: 2354-2360). Since the HIV-1 M group began its expansion in humans roughly 70 years ago (Korber et al., Retroviral Immunology, Pantaleo et al., eds., Humana Press, Totowa, N.J., 2001, pp. 1-31), it has diversified rapidly (Jung et al., (2002) Nature 418: 144). The HIV-1 M group consists of a number of different clades (also known as subtypes) as well as variants resulting from the combination of two or more clades, known as circulating recombinant forms (CRFs). Subtypes are defined as having genomes that are at least 25% unique (AIDS epidemic update, December 2002). Eleven clades have been identified and a letter designates each subtype. When clades combine with each other and are successfully established in the environment, as can occur when all individual is infected with two different HIV subtypes, the resulting virus is known as a CRF. Thus far, roughly 13 CRFs have been identified. HIV-1 clades also exhibit geographical preference. For example, Clade A, the second-most prevalent clade, is prevalent in West Africa, while Clade B is common in Europe, the Americas and Australia. Clade C, the most common subtype, is widespread in southern Africa, India and Ethiopia (AIDS epidemic update, December 2002). This genetic variability of HIV creates a scientific challenge to vaccine development.

An infectious HIV particle consists of two identical strands of RNA, each approximately 9.2 kb long, packaged within a core of viral proteins. This core structure is surrounded by a phospholipid bilayer envelope derived from the host cell membrane that also includes virally-encoded membrane proteins (Abbas et al., Cellular and Molecular Immunology, 4th edition, W.B. Saunders Company, 2000, p. 454). The HIV genome has the characteristic 5′-LTR-gag-pol-env-LTR-3′ organization of the retrovirus family. Long terminal repeats (LTRs) at each end of the viral genome serve as binding sites for transcriptional regulatory proteins from the host and regulate viral integration into the host genome, viral gene expression, and viral replication. The HIV genome encodes several structural regulatory proteins. The gag gene encodes core structural proteins of the nucleocapsid core and matrix. The pol gene encodes RT, integrase, and viral protease enzymes required for viral replication. The tat gene encodes a protein that is required for elongation of viral transcripts. The rev gene encodes a protein that promotes the nuclear export of incompletely spliced or unspliced viral RNAs. The vif gene product enhances the infectivity of viral particles. The vpr gene product promotes the nuclear import of viral DNA and regulates G2 cell cycle arrest. The vpu and nef genes encode proteins that down regulate host cell CD4 expression and enhance release of virus from infected cells. The env gene encodes the viral envelope glycoprotein that is translated as a 160-kilodalton (kDa) precursor (gp160) and cleaved by a cellular protease to yield the external 120-kDa envelope glycoprotein (gp120) and the transmembrane 41-kDa envelope glycoprotein (gp41), which are required for the infection of cells (Abbas, pp. 454-456).

HIV infection initiates with gp120 on the viral particle binding to the CD4 and chemokine receptor molecules (e.g., CXCR4, CCR5) on the cell membrane of target cells such as CD4+ T-cells, macrophages and dendritic cells. The bound virus fuses with the target cell and reverse transcribes the RNA genome. The resulting viral DNA integrates into the cellular genome, where it directs the production of new viral RNA, and thereby viral proteins and new virions. These virions bud from the infected cell membrane and establish productive infections in other cells. This process also kills the originally infected cell. HIV can also kill cells indirectly because the CD4 receptor on uninfected T-cells has a strong affinity for gp120 expressed on the surface of infected cells. In this case, the uninfected cells bind, via the CD4 receptor-gp120 interaction, to infected cells and fuse to form a syncytium, which cannot survive. Destruction of CD4+ T-lymphocytes, which are critical to immune defense, is a major cause of the progressive immune dysfunction that is the hallmark of AIDS disease progression. The loss of CD4+ T cells seriously impairs the body's ability to fight most invaders, but it has a particularly severe impact on the defenses against viruses, fungi, parasites and certain bacteria, including mycobacteria.

One hope for controlling the AIDS pandemic is the development of a safe, effective, accessible prophylactic HIV vaccine. At present, acceptable HIV vaccines may seem only partially effective when measured against traditional vaccine standards. For example, an acceptable HIV vaccine may be effective just for some people or for a limited time period. Alternatively, such a vaccine may not stop HIV infection, but thwart progression to AIDS in immunized individuals who later contract the virus. While such vaccines may be less than ideal, partial protection can be a valuable public health tool until better products are developed. Indeed, the Salk polio vaccine, introduced in 1955, was only 60% effective, but managed to bring polio in the U.S. under significant control.

Moreover, traditional approaches to vaccine development, such as immunization with live attenuated virus, killed virus or viral subunits, are not proving feasible for HIV. For example, in the macaque-SIV model, live attenuated vaccines cause persistent infection, with some macaques developing AIDS. As another example, neutralizing antibodies to gp120 exist for laboratory-adapted HIV isolates (Berman et al., (1990) Nature 345: 622-625; Fultz et al., (1992) Science 256: 1687-1690). However, it has been difficult to generate effective neutralizing antibodies to clinical isolates of virus. Combinations of traditional and new approaches with novel immunogens designed to elicit humoral and/or cellular immunity may prove necessary and are being actively sought.

An acceptable and effective HIV vaccine may need to stimulate both neutralizing antibodies and cell-mediated immune responses at both systemic and mucosal sites. With the difficulties encountered for neutralizing antibodies, another approach to HIV vaccine development is to induce cell-mediated immune responses. Such responses are predominantly mediated by cytotoxic T lymphocytes (CTLs). CTLs, also known as CD8+ T-cells, participate in an organism's defense in at least two different ways: by killing virus-infected cells and by secreting a variety of cytokines and chemokines that directly or indirectly contribute to the suppression of virus replication. The induction and maintenance of strong CD8+ T cell responses require “help” provided by CD4+ T-lymphocytes (helper T-cells).

CTL recognize peptides that originate from both surface and inner structural and non-structural HIV proteins. Unlike antibodies, they cannot prevent cell-free HIV from infecting host cells. Therefore, the vaccine-induced prophylactic CTL will have to act fast. For that, they may have to be in sufficient numbers, which may or may not require persistent vaccine stimulation or regular re-vaccinations. Preferably, vaccine-induced CTLs cells should recognize early and/or abundant HIV proteins of the transmitting virus/clade, target multiple CTL epitopes in functionally conserved protein regions to make it hard for HIV to escape, and kill target cells efficiently.

CTLs also play specific roles in the control of HIV and SIV infections (McMichael et al, (2001) Nature 410: 980-987). HIV-specific CTLs appear shortly after infection and peak a few days after the primary viremia (Ogg et al., (1998) Science 279:2103-2106). As HIV-specific CTLs reach maximal numbers, up to 10% of all CD8+ T-cells, the level of virus falls. Interestingly, viremia does not decrease when macaques infected with SIV are treated with anti-CD8 antibodies during acute infection. (Matano et al., (1998) J. Virol. 72:164-169; Schmitz et al., (1999) Science 283: 857-860; Jin et al., (1999) J. Exp. Med. 189:991-998; Lifson et al., (2001) J. Virol. 75:10187-10199). Further, infusion of anti-CD8 antibodies during chronic infection leads to an immediate increase in viremia, which falls once CD8+ T-cells return. Additional evidence of CTLs playing a role in HIV infection comes from a Nairobi female sex-worker cohort who has been highly exposed to HIV, yet remain persistently seronegative. The cohort expresses neither defective virus receptor genes, nor anti-HIV immunoglobulin G. However, HIV resistance in this group is associated with systemic HIV-1-specific helper T-cells and CTLs as well as cervical HIV-1-specific CTLs, which were not present in lower-risk control women (Kaul et al., (2001) J. Clin. Invest. 107:1303-1310).

To induce CTL, a prime-boost immunization strategy using plasmid DNA encoding an immunogen as a priming immunization, followed by a boosting immunization with a recombinant virus encoding the same immunogen, has demonstrated efficacy to stimulate CD8+ T cell responses in mice (Hanke et al., (1998a) Vaccine 16:439-445; Schneider et al., (1998) Nat. Med. 4: 397-402; Kent et al., (1998) J. Virol. 72:10180-10188). This strategy has been confirmed and extended for non-human primates (Hanke et al, (1999) J. Virol 73:7524-7532; Allen et al., (2000a) J. Immunol. 164: 4968-4978; Amara et al., (2001) Science 292:69-74; Allen et al., (2002) J. Virol. 76:10507-10511; Shiver et al., (2002) Nature 415:331-335) and humans (McConkey et al., (2003) Nat. Med. 9:729-35). WO 98/56919 discloses a prime-boost immunization strategy to generate a CTL-mediated immune response against malarial and other antigens, such as viral and tumour antigens. This immunization strategy uses priming and boosting compositions, which deliver the same CTL epitope in different vectors, where the vector for the boosting composition is a replication-defective poxvirus vector.

In particular, successive immunization with plasmid DNA and modified vaccinia virus Ankara (MVA) vector expressing a common immunogen induce T cell responses (Hanke 1998a; Schneider; Hanke 1999; Allen 2000a; Amara; Allen 2002; Shiver). For clinical trials, the immunogen HIVA was constructed. HIVA contains consensus HIV Clade A gag p24/p17 sequences and a string of selected Clade A CTL epitopes (WO 01/47955; Hanke et al., Nat. Med. 6:951-55, 2000; Hanke et al., Vaccine 20:1995-1998, 2002a). The HIVA DNA and MVA vaccines were shown to be immunogenic in mice (Hanke 2000; Hanke et al., (2003) J. Gen. Virol. 84:361-368; Hanke et al., (2002b) Vaccine 21:108-114) and rhesus macaques (Wee et al., (2002) J. Gen. Virol. 83:75-80) and has lead to the first HIV-1 Clade A-derived vaccine tested in humans. The HIVA immunogen does not contain the envelope (env) and focuses solely on the induction of cell-mediated immune responses, allowing assessment of their role in the protection against HIV infection and/or disease and addition of a component to stimulate neutralizing antibody formation when available.

Another aspect of vaccine development is to find formulations capable of inducing CTL responses specific for multiple HIV epitopes. Such vaccines could make it relatively difficult for HIV to escape and would have a better chance to suppress HIV replication. Theoretically, several smaller immunogens delivered individually by separate vaccine vectors would be advantageous over one large multigenic protein expressed from a single vector, because the former immunogens may reach separate antigen-presenting cells and each induce at least one immunodominant response (Singh et al., J. Immunol. 168:379-391). With a multigenic protein, unless cross-priming plays a role in immune stimulation, each component is produced by one cell and thus competes with the others for presentation. Hence, a balance is needed between the breadth of elicited immune responses and practicalities of vaccine development and production, the former increasing and the latter decreasing the number of vaccine components.

Citation or identification of any document in this application is not an admission that such document is available as prior art to the present invention.

### SUMMARY OF THE INVENTION

The AFPs of the present invention may be non-naturally occurring proteins that may comprise multiple HIV domains and one or more human CTL epitopes associated with long term nonprogression to AIDS. In particular, the AFPs of the invention may comprise a) an HIV tat domain which lacks the nuclear localization signal, the integrin interaction domain and transactivation activity; b) one or more HIV reverse transcriptase domains, each of which lacks polymerase activity; c) an HIV nef domain which can not be myristylated; d) two CTL-rich domains from HIV gp41, wherein the first domain consists essentially of amino acids 699-742 of SEQ ID NO: 1 or the equivalent amino acids from gp41 of an HIV isolate or an HIV consensus sequence, and wherein the second domain consists essentially of amino acids 743-843 of SEQ ID NO: 1 or the equivalent amino acids from gp41 of an HIV isolate or an HIV consensus sequence; and e) one or more human HIV CTL epitopes associated with long term non-progression to AIDS (also referred to herein as the “human CTL epitope”). The AFPs stimulate an HIV-specific CTL response. Moreover, the domains can be, but are not necessarily selected so that the AFP stimulates an immune response to a pre-determined HIV clade.

These domains can be present from amino (N) to carboxyl (C) terminus of the AFP in any order that does not recreate a naturally-occurring HIV protein or otherwise create a protein encoded in an HIV genome.

In one embodiment, the order of domains, from N to C terminus, is HIV tat domain, first HIV reverse transcriptase domain, HIV nef domain, second HIV reverse transcriptase domain, the first CTL-rich domain from HIV gp41, the second CTL-rich domain from HIV gp41 and the human HIV CTL epitope. The domains of the AFPs are optionally, and independently, separated from each other with intervening sequences. The amino acid sequences for each of the HIV tat, reverse transcriptase, nef, and CTL-rich env domains and each human HIV CTL epitope are preferably from an HIV consensus sequence for the same HIV Clade, and more preferably from an HIV Clade A consensus sequence.

The AFPs of the invention can optionally comprise one or more additional domains useful for monitoring expression levels of the AFP in cells or laboratory animals and/or immune responses to the AFP in laboratory animal, such as mice, non-human primates, rats, rabbits and the like.

Preferred AFPs of the invention include an AFP comprising amino acids 1-843 of SEQ ID NO: 1 as well as an AFP comprising amino acids 1-871 of SEQ ID NO: 1. The latter protein is known as RENTA and described below. A schematic diagram of RENTA is shown in FIG. 1A; the amino acid sequence (SEQ ID NO: 1) and nucleotide sequence (in SEQ ID NO: 2) of RENTA is shown in FIGS. 2 and 3, respectively.

Another aspect of the invention provides isolated nucleic acids encoding an AFP of the invention and expression vectors comprising a nucleic acid encoding an AFP of the invention operably linked to at least one nucleic acid control sequence. Such expression vectors include, but are not limited to, plasmid vectors (for prokaryotic and/or eukaryotic cells), viral vectors, insect vectors, yeast vectors and bacterial vectors (including Mycobacterial vectors and Bacillus vectors). Preferred vectors include pTHr (Hanke et al., Vaccine 16:426-435, 1998b; Hanke 2000) and modified vaccinia Ankara (MVA), which is a vaccinia vector. The codon usage for the AFP coding sequence is preferably that of highly expressed genes of the target organism or host cell in which the expression vector is being used, i.e., the organism or cell in which mRNA translation occurs. When the expression vectors or nucleic acids are used for immunization in humans, the codon usage is preferably that of highly expressed human genes. Preferred expression vectors of the invention with an encoded AFP are pTHr.RENTA and MVA.RENTA.

The invention also includes host cells containing an expression vector of the invention as well as methods of preparing AFPs by culturing those host cells for a time and under conditions sufficient to express the AFP, and recovering the AFP.

Yet another aspect of the invention relates to methods for expressing an AFP of the invention in animal cells by introducing an expression vector of the invention into the animal cells and culturing those cells under conditions sufficient to express said AFP. The expression vector can be introduced by any appropriate method including, but not limited to, transfection, transformation, infection and the like.

A further aspect of the invention relates to methods for introducing into and expressing an AFP of the invention in an animal by delivering an expression vector of the invention into the animal to thereby obtain expression of the AFP in the animal. Any delivery method can be used including intramuscular, intravenous, intradermal, mucosal, topical or other delivery method, such as the Powderject method (a needle-less particle delivery system to the skin) for delivering expression vector immunogens or protein immunogens.

Still another aspect of the invention provides methods for inducing an immune response in an animal by delivering an expression vector of the invention into the animal, so that the encoded AFP is expressed at a level sufficient to stimulate an immune response to the AFP. Similarly, the invention provides methods to induce an immune response in an animal by delivering the AFP itself into the animal in an amount sufficient to stimulate an immune response to AFP. Any delivery method can be used, e.g., as described in the preceding paragraph. Any combination of immunogens of the invention (e.g., expression vectors or proteins) can be used with any immunization schedule to induce an immune response to HIV.

Yet another aspect of the invention relates to methods of stimulating an immune response against HIV in a human by administering an AFP of the invention, a nucleic acid of the invention and/or an expression vector of the invention one or more times to a subject, wherein the AFP is administered in an amount or expressed at a level sufficient to stimulate an HIV-specific CTL immune response in said subject. Such immunizations can be repeated multiple times at time intervals of at least 2 or more weeks in accordance with a desired immunization regime or strategy. The method can be used in combination with other HIV immunogens, including proteins, expression vectors and the like. When used in combination, the other HIV immunogens can be administered at the same time or at different times as part of an overall immunization regime, e.g., as part of a prime-boost regimen or other immunization protocol. Many other HIV immunogens are known in the art, one such preferred immunogen is HIVA, which can be administered as a protein, on a plasmid (e.g., pTHr.HIVA) or in a viral vector (e.g., MVA.HIVA). A schematic representation of HIVA is shown in FIG. 1B.

For example, one method of stimulating an immune response against HIV in a human subject comprises administering at least one priming dose of an HIV immunogen and at least one boosting dose of an HIV immunogen, wherein the immunogen in each dose can be the same or different, provided that at least one of the immunogens is an AFP of the invention, a nucleic acid encoding an AFP of the invention or an expression vector encoding an AFP of the invention, and wherein the immunogens are administered in an amount or expressed at a level sufficient to stimulate an HIV-specific immune response in the subject. The HIV-specific immune response can include an HIV-specific CTL immune response. Again, such immunizations can be done at intervals, preferably of 2 weeks or more, including 6 weeks or longer intervals.

In one embodiment, pTHr.RENTA is administered one or more times as the priming dose. In another embodiment, MVA.RENTA is administered one or more times as the boosting dose, with or without the priming dose having been pTHr.RENTA.

A still further aspect of the invention provides an immunogenic composition comprising an AFP of the invention, a nucleic acid encoding the AFP or an expression vector encoding the AFP in admixture with a pharmaceutically acceptable carrier. The immunogenic composition is useful as formulated or as a component for prophylactic or therapeutic vaccination against HIV. The composition can optionally include an adjuvant such as mineral salts, polynucleotides, polyarginines, ISCOMs, saponins, monophosphoryl lipid A, imiquimod, CCR-5 inhibitors, toxins, polyphosphazenes, cytokines, immunoregulatory proteins, immunostimulatory fusion proteins, co-stimulatory molecules, and combinations thereof. Mineral salts include, but are not limited to, AIK(SO₄)₂. AlNa(SO₄)₂, AlNH(SO₄)₂, silica, alum, Al(OH)₃, Ca₃(PO₄)₂, kaolin, or carbon. Useful immunostimulatory polynucleotides include, but are not limited to, CpG oligonucleotides with or without immune stimulating complexes (ISCOMs), CpG oligonucleotides with or without polyarginine, poly IC or poly AU acids. Toxins include cholera toxin. Saponins include, but are not limited to, QS21, QS17 or QS7. An example of a useful immunostimulatory fusion protein is the fusion protein of IL-2 with the Fc fragment of immunoglobulin. Useful immunoregulatory molecules include, but are not limited to, CD40L and CD1a ligand. Cytokines useful as adjuvants include, but are not limited to, IL-2, IL-4, GM-CSF, IL-12, IGF-1, IFN-α, IFN-β, and IFN-γ. Combinations of adjuvants can also be used.

These and other embodiments are disclosed or are obvious from and encompassed by, the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIGS. 1A and 1B present a schematic representation of RENTA and HIVA, respectively. RENTA is described herein. HIVA contains portions of the HIV gag, p17 and p24 proteins from a consensus sequence for HIV Clade A and a string of Clade A CTL epitopes. HIVA is described in WO01/47955. Similar to RENTA, HIVA also has one monkey CTL epitope (Mamu), one mouse CTL epitope (P18-I10, discussed below and indicated as H-2 in the drawing) and a monoclonal antibody (mAb) epitope (Pk).

FIG. 2 provides the amino acid sequence (SEQ ID NO: 1) of RENTA in one letter amino acid code. The HIV proteins from which the amino acid sequences originate are marked and also indicated by upper case. Amino acids in lower case originate from the restriction enzyme linker.

FIG. 3 provides the nucleic acid sequence (SEQ ID NO: 2) of the HindIII-XbaI restriction fragment containing the RENTA open reading frame (ORF). The sequences in lower case identify the restriction enzyme linkers used in the construction of RENTA. The first codon (ATG) in RENTA begins at nucleotide 25.

FIG. 4 depicts the HIV tat domain in RENTA and corresponding consensus sequences from Clades A-A 1-A2, B, C and D. These consensus sequences, and those depicted in FIGS. 5-8, were obtained from manual alignments of sequences in the Los Alamos HIV database. For all sequences, a dash indicates sequence identity and an internal asterisk (*) or blank space (at the ends) indicates the corresponding amino acid is missing.

For the HIV tat domain, the consensus sequences have been altered so that the corresponding domains lack the nuclear localization signal, the integrin interaction domain and transactivation activity. The solid dot (•) indicates the NLS deletion and the bold, lower case g indicates point mutations associated with loss of transactivation activity (i.e., by changing cysteine to glycine at those positions).

FIG. 5 depicts the carboxyl-terminal HIV reverse transcriptase (C-RT) domain in RENTA and corresponding consensus sequences from Clades A-A1-A2, B, C and D.

FIG. 6 depicts the HIV nef domain in RENTA and corresponding consensus sequences from Clades A-A 1-A2, B, C and D.

FIG. 7 depicts the amino-terminal HIV reverse transcriptase (N-RT) domain in RENTA and corresponding consensus sequences from Clades A-A 1-A2, B, C and D.

FIG. 8 depicts the first HIV env domain (amino acids 557-600 of gp41, which correspond to amino acids 699-742 of SEQ ID NO: 1) and second HIV env domain (amino acids 765-856 of gp41 which correspond to amino acids 743-843 of SEQ ID NO: 1) in RENTA and the corresponding consensus sequences from Clades A-A 1-A2, B, C and D.

FIG. 9 shows an immunoblot of polypeptides from DNA-transfected and MVA-infected cells using the anti-Pk mAb for detection. Relative molecular masses of protein markers are indicated.

FIG. 10 graphically illustrates the amount of ³H-acetylchloramphenicol produced as a function of time in a standard chloramphenicol acetyltransferase (CAT) assay for human 293T cells transiently transfected with LTR-CAT plasmid alone (white, left box); LTR-CAT and CMV-Tat plasmids (grey, middle box) or LTR-CAT and pTHr.RENTA plasmids (black, right box).

FIG. 11 illustrates the surface expression of HLA Class 1 molecules (top panels) and CD4 molecules (bottom panels) as assessed by mAb staining and fluorescence-activated cell sorting (FACS) of human peripheral blood mononuclear cells (PBMCs) expressing GFP alone (left panels), GFP and wild type Nef (center panels) or GFP and RENTA (right columns).

FIG. 12A graphically illustrates the percentage of specific lysis as a function of effector target cell ratio in a ⁵¹Cr-release assay for mice immunized with pTHr.RENTA (left panel) or MVA.RENTA (right panel) using pb9 peptide-pulsed (solid circle) or unpulsed (open circle) target cells.

FIG. 12B graphically illustrates the percentage of specific lysis as a function of effector:target cell ratio in a ⁵¹Cr-release assay for mice immunized according to the DNA prime-MVA boost regime of Example 5 for HIVA alone (top left panel), RENTA alone (top right panel) or mixed HIVA/RENTA (bottom panels). The CTL responses against an HIVA CTL epitope (P18-I10) are shown in the two left panels by diamonds for P18-I10 peptide-pulsed (closed) or unpulsed (open) target cells. The CTL responses against a RENTA CTL epitope (pb9) are shown in the two right panels by circles for pb9 peptide-pulsed (closed) or unpulsed (open) target cells.

FIG. 12C graphically illustrates the results of an ELISPOT assay and shows relative IFNγ production (as spot-forming units; SFU) stimulated by the pb9 peptide for RENTA (hatched boxes) or by the P18-I10 peptide for HIVA (open boxes) for each of the three prime-boost regimens of Example 5, from left to right, RENTA only, HIVA only or mixed HIVA/RENTA.

FIG. 13 graphically illustrates the effects of physically separating immunizations in a DNA prime-MVA boost protocol as assessed using an intracellular IFN-γ staining assay (panel A), an H-2D^{d}/P18-I10 tetramers assay (panel B), an IFN-γ ELISPOT assay (panel C), and a ⁵¹Cr-release assay (panel D). Mice received immunizations as follows: pTHr.HIVA DNA and MVA.HIVA into the left leg and pTHr.RENTA DNA and MVA.RENTA into the right leg (SS); each plasmid into a separate leg and mixed MVAs into both legs (SM); mixed plasmids into both legs and each MVA into a separate leg (MS); or mixed plasmids and mixed MVAs into both legs (MM). The details of the assays, results and abbreviations are provided in Example 7. Panel A shows the percentage of CD8+ cells producing IFN-y for the indicated peptides or peptide pools. Panel B shows the percentage of CD3+ and CD8+ cells reactive with H-2D^{d}/P8-I10 tetramers. Panel C shows relative IFN-γ production as SFU in the ELISPOT assay for the indicated peptides. Panel D shows the ⁵¹Cr-release assay using splenocytes from prime-boost regimes SS (grey circles), SM (grey squares), MS (black circles) and MM (black squares) with target P815 cells unpulsed (open) or pulsed (solid) with the peptide indicated at the top of the graph.

FIG. 14A shows FACS plots with the percentage of CD8+ cells reactive with Mamu-A*01/Tat tetramers for Monkeys 1 and 2 immunized with pTHr.HIVA and pTHr.RENTA only as described in Example 8 and blood drawn at week 16.

FIG. 14B shows FACS graphs with the percentage of CD8+ cells reactive with Mamu-A*O1/Tat tetramers (top panels) or reactive with Mamu-A*01/Gag tetramers (bottom panels) for Monkeys 1 and 5 immunized with pTHr.HIVA and pTHr.RENTA (as primes) followed by MVA.HIVA and MVA.RENTA (as boosts) as described in Example 8 and blood drawn at week 22.

FIG. 14C shows relative IFN-γ production as SPU in the ELISPOT assay for the indicated peptides using splenocytes from Monkey 1 immunized and bled as for FIG. 14B.

FIG. 14D shows a standard ⁵¹Cr-release assay after a 2-week peptide restimulation in vitro of PBMC (week 26). Dark blue-Tat peptide; light blue-Gag peptide; orange and red —HIVA peptide pools 1+2 and 3+4, respectively; and dark green, light green and purple —RENTA peptide pools 1+3, 4+5, and 2+6, respectively.

FIG. 15 shows the expression of the RENTA chimeric protein in human 293T cells from pTHr.RENTA (a-d, g and h) and MVA.RENTA (i) was detected using immunofluorescence and mAb to the indicated subdomains. For (a-d), the nuclei are shown in blue, Tat, RT and PK in red and Nef in green. Colocalization of a related fusion protein containing unmutated Tat (red anti-Pk; e and f) and RENTA (red anti-Pk; g and h) with lysosomal/late endosomal marker (green; e and g) and the Golgi matrix protein (green; f and h) in transfected 293T cells. The arrows indicate the presence of a recombinant protein in the nucleus, consistent with the NLS of unmutated Tat.

FIG. 16 are representative examples of intracellular cytokine and H-2D^{d}/P18-I10 tetramer staining of mouse splenocytes. Panel (a) shows IFN-γ production by splenocytes isolated from a mouse immunized using mixed HIVA and RENTA vaccines in a DNA prime and MVA boost regimen, and a naïve mouse as a control. The breadth of vaccine-elicited immune responses was assessed by using individual epitope peptides or overlapping peptide pools across RENTA indicated above. Inserted numbers indicate IFN-γ producing cells as a percentage of CD8+ splenocytes. Panel (b) shows the effect of separate or mixed deliveries of the HIVA and RENTA vaccines on immunogenicity. Inserted numbers give the percentage of CD3+ and CD8+ splenocytes reactive with the tetramer.

FIG. 17 shows HIVA and RENTA co-immunization in mice. Groups of BALB/c mice were co-immunized with increasing doses of mixed vaccines: A—Naïve; B—6.25 μg DNA prime—2×10³ pfu MVA boost; C—12.5 μg DNA prime—2×10⁴ pfu MVA boost; D—25.0 μg DNA prime—2×10⁵ pfu MVA boost; E—50.0 μg DNA prime—2×10⁶ pfu MVA boost; F-100 μg DNA prime—2×10⁷ pfu MVA boost. Red and blue are MHC Class I-restricted peptide epitopes in HIVA and RENTA, respectively, designated H, P, G1, M, RT2, and E.

FIG. 18 shows responses in Monkey 1 on week 36 (28 weeks after the first MVA administration). T-cell responses to both novel and previously identified CTL epitopes were identified in frozen PBMC samples restimulated with indicated peptides in an intracellular cytokine staining assay. Red and black epitopes are derived from HIVA and RENTA immunogens, respectively.

FIG. 19 depicts HIV-specific responses induced by a combined HIVA+RENTA vaccination on week 70. Fresh PBMC showed vaccine-induced T-cell responses in an IFN-γ ELISPOT assay one year after vaccine administration. Monkey 4—Judd; monkey 5—Jill; monkey 1—Joe; monkey 2—Jig. Red and black epitopes are derived from HIVA and RENTA immunogens, respectively.

### DETAILED DESCRIPTION

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The present invention relates to AFPs for promoting immune responses to HIV in a human subject. These AFPs are non-naturally occurring proteins that comprise multiple HIV domains and one or more human CTL epitopes associated with long term non-progression to AIDS. The AFPs of the invention can optionally comprise one or more additional domains useful for monitoring expression levels of an AFP in cells or laboratory animals and/or immune responses to the AFP in laboratory animals.

In particular, the AFPs of the invention comprise (a) an HIV tat domain which lacks the nuclear localization signal, the integrin interaction domain and transactivation activity; (b) one or more HIV reverse transcriptase domains, each of which lacks polymerase activity; (C) an HIV nef domain which cannot be myristylated; (d) two CTL-rich domains from HIV gp41, wherein one domain consists essentially of amino acids 699-742 of SEQ ID NO: 1 or the equivalent amino acids from HIV gp41 or an HIV gp41 consensus sequence, and wherein the second domain consists essentially of amino acids 743-843 of SEQ ID NO: 1 or the equivalent amino acids from HIV gp41 or an HIV gp41 consensus sequence; and (e) one or more human HIV CTL epitopes associated with long term non-progression to AIDS. The amino acid sequence of the HIV domains can he selected so that the AFP predominantly stimulates an immune response to a pre-determined HIV Clade. For example, if an immune response against HIV Clade A is desired, then the amino acid sequences for Tat, RT, Nef, and the CTL-rich domains of gp41 are preferably the Clade A consensus sequences for each of those proteins. While not required, the human CTL epitopes associated with long term non-progression to AIDS are preferably active against the same pre-determined Clade.

More particularly, an “artificial fusion protein” or “AFP” as used herein is a protein or polypeptide (these terms are used interchangeably) which does not naturally occur in nature, i.e., AFPs are the product of a design process and the entire AFP as designed is not naturally encoded in the genome of an organism. An AFP of the invention must have at least two distinct protein domains arranged in a non-naturally occurring manner, i.e., the two domains are arranged (or fused together) in a manner not normally found in a single protein. For domains originating from different proteins, the arrangement (or order of joining) is flexible. If the two domains are from the same protein or from a single polyprotein, such as a viral polyprotein, the domains are joined together in a manner to provide a primary linear structural arrangement that differs from the original primary structure associated with those domains, as they are encoded in the protein is in the genome of the organism from which the domains are derived. For example, contiguous domains from a single protein can be joined in reverse order or can be separated by an intervening domain. For example, an AFP could be made by figuratively cutting a protein in half and reordering the coding sequence for (or fusing) the fragments so that the sequence normally found at the carboxy end of the protein is now at the amino terminus of the AFP and the original amino-terminal amino acid is in the middle of the protein.

The domains of the AFPs can be joined by any means, including, without limitation, by covalent bonds, such as a peptide bond or via insertion of a chemical linker, or by non-covalent bonds, such as an ionic bond. Preferably, the domains of the AFPs are joined by covalent bonds. As used herein, “domain” means a region or sequence of amino acids from a protein or polypeptide without regard to whether that region or sequence forms a particular structural or functional unit. However, the selection of particular amino acids as a domain does not preclude that domain from also being a structural and/or functional unit of the protein or polypeptide or from having been selected on the basis of its structure or function.

The size of the domain can vary from a few (less than 10) to many hundreds of amino acids, with the actual domain size based on the reason that particular domain is included in the AFP. For example, a domain that serves as a spacer may range from 2-3 amino acids to 10-15 amino acids, with the exact number of amino acids determined as needed, e.g., to facilitate cloning sites, to avoid frameshifts in the reading frames of the coding sequences, to provide a particular distance between domains, or for any combination of these or other reasons. As another example, a domain whose function is to encode CTL epitopes may range from 5-12 amino acids if a single epitope is encoded, or may be several hundred amino acids if multiple epitopes are encoded. If desired, a domain in the AFP can consist of an entire protein or modified versions of an entire protein, again as dictated by the reason for including that domain in the AFP.

The amino acid sequence of a domain is determined by the nature of the individual domain of the present invention and described in detail below. In this regard, those sequences include naturally-occurring sequences, modified sequences, consensus sequences and the like. Sequence modifications can be achieved by deleting, inserting or changing one or more amino acids. New domains can be made by changing the normal arrangement of amino acids, e.g., by transposing different parts of the protein.

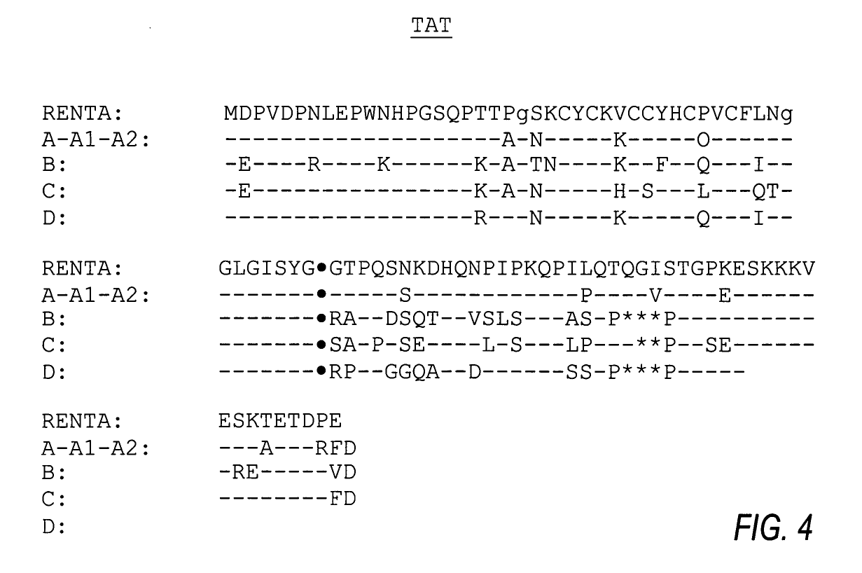

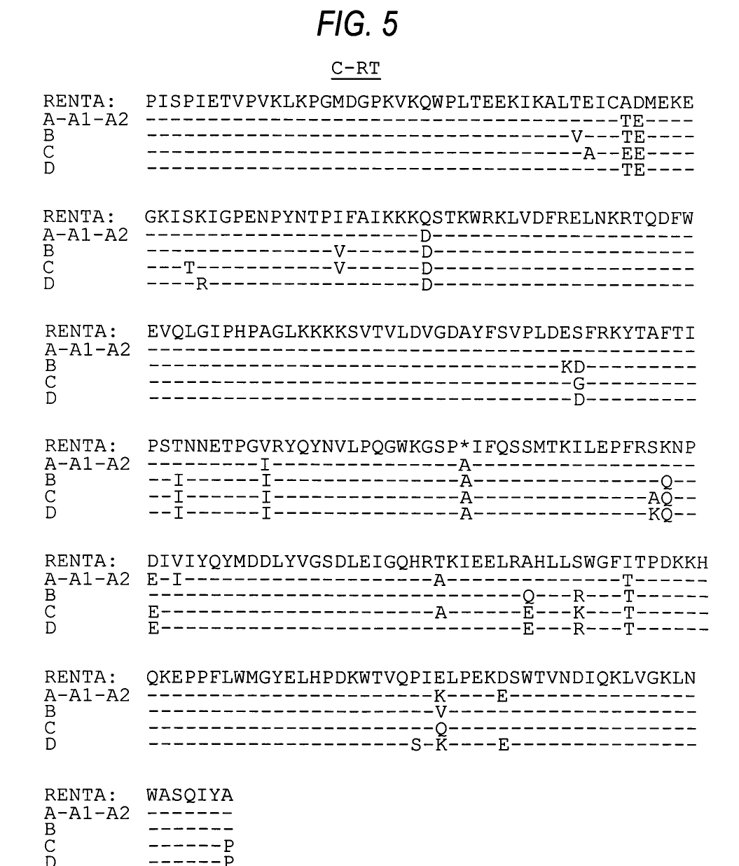

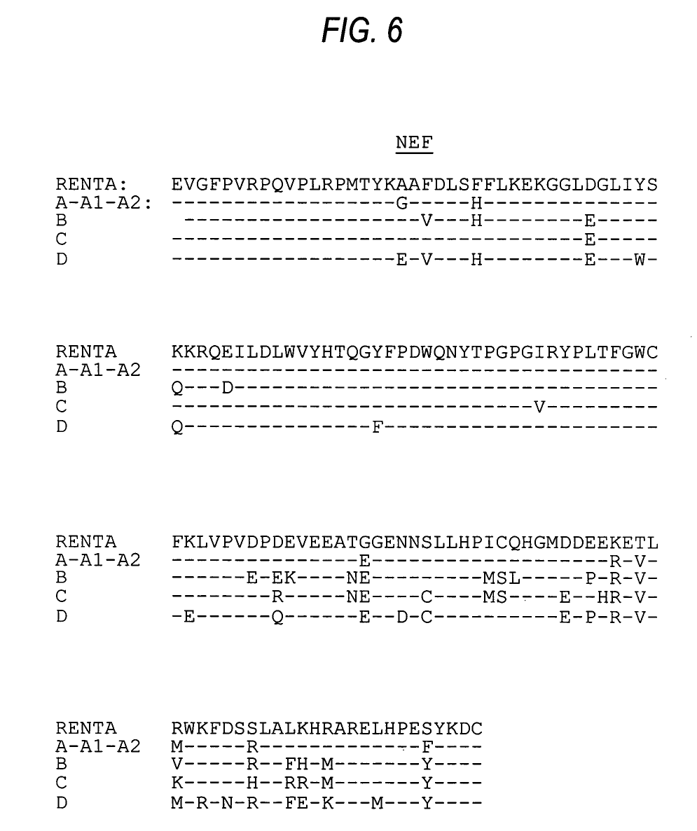

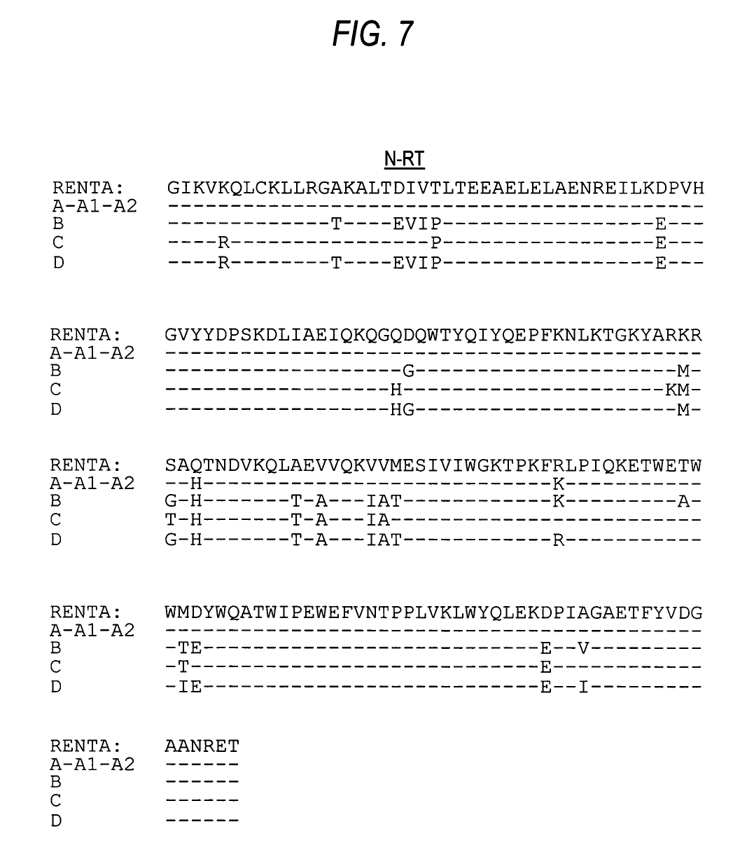

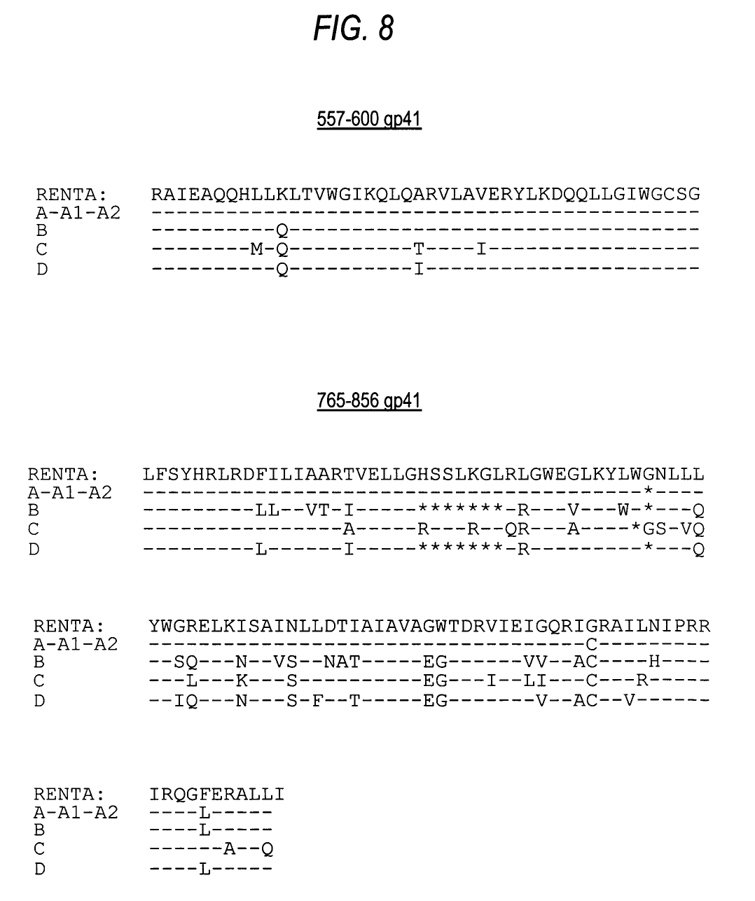

The amino acid sequence for the reverse transcriptase, env/gp41, nef, and tat domains in the AFPs of the invention can be from a consensus sequence for a specific Clade to preferentially generate an immune response to that specific Clade. Alternatively, the amino acid sequences of the domains can be selected to generate an immune response against any of the other HV clades, by using amino acid sequences conserved within, and characteristic of, the selected Clade. For example, consensus sequences as of 2002 across clades A-A 1-A2, B, C and D for domains of HIV reverse transcriptase, gp41, tat and nef in RENTA as used in the present invention are provided in FIGS. 4 through 8. HIV Clades include clades A, B, C, D, H, F, G, H, I, J, and K. Consensus sequences from CRFs can also be used.

The simplest form of a consensus sequence is created by picking the most frequent amino acid at each position of a protein in a set of aligned protein sequences. Thus, as the number of proteins being compared increases, the consensus sequence can change. The consensus sequence for HIV proteins from different clades is regularly updated by the Los Alamos HIV database and is readily available to the public. While these compilations may evolve over time as additional isolates of HIV are analyzed and as Clade groupings are altered, this evolution does not affect the use of consensus sequences in the present invention. Any of these published consensus sequences or any consensus sequence derived from a desired group of sequences can be used in the invention.

To select the equivalent or corresponding amino acids for the domains of the invention (these terms are used interchangeably), one of skill in the art aligns the candidate HIV isolate or consensus sequence with the indicated amino acids of SEQ ID. NO: 1 and thereby determines the corresponding sequence, making allowances for deletions and insertions of amino acids in that region of sequence. It is well know that such alignments may not yield precisely the same length amino acid sequences due to well known HIV variation. Consequently, the domains for equivalent sequences generally vary in size from 1 to 15 amino acids (or fewer, preferably from 1-10 or 1-5 amino acids and more preferably 1, 2 or 3 amino acids) to accommodate small insertions and deletions. Such insertions and deletions can be occur within or at the ends of the equivalent sequence, provided that such length alterations are those one of skill in the art would obtain in maximizing the alignment between the candidate HIV sequence and the indicated portions of SEQ ID NO: 1. Alignment techniques, including manual methods or computerized algorithms, are known to those of skill in the art.

The domains of the AFPs can be arranged in a variety of different ways (e.g., in a linear order from N- to C-terminus or via chemical crosslinking) without significantly affecting the immunogenic character of the AFP. Accordingly, the AFPs can have the domains arranged in any order that preserves immunogenicity, preserves the required characteristics of the individual domains (e.g., abolishes the relevant biological activity), and does not recreate a naturally-occurring protein.

The AFPs can be synthesized by conventional chemical techniques, such as solid phase synthesis or produced by recombinant DNA technology, preferably the latter. Individually-produced domains can be purified and joined by chemical cross-linking or any other method known in the art. Methods of synthesis, recombinant DNA techniques to produce proteins and chemical cross-linking methods are well known to those of skill in the art. Hence, the invention includes methods of preparing AFPs by culturing a host cell containing an expression vector of the invention (see below) for a time and under conditions sufficient to express the AFP, and recovering the AFP. Methods useful to recover, and/or purify the AFP to homogeneity can be determined by those of skill in the art.

The domains and intervening sequences of the AFPs of the invention are described in detail below under headings A-G. Heading H provides a description of RENTA, a preferred embodiment of the present invention.

The HIV tat domain of the AFPs lacks the nuclear localization signal, the integrin interaction domain and transactivation activity of the HIV Tat protein (“Tat”), but can otherwise contain the remainder of Tat. Any HIV tat domain that lacks the preceding activities and otherwise retains significant CTL-inducing ability can be used in the AFPs of the invention and is thus an HIV tat domain of the invention. Preferably, at least 75% of the Tat protein sequence is present in the HIV tat domain. Provided that the indicated biological activities of Tat are lacking, there can be from about 80%, 85%, 90% or 95% of the full Tat protein sequence in the HIV tat domain.

To produce the HIV tat domain of the invention, all or part of the nuclear localization signal sequence (“NLS”) of Tat is deleted. The Tat NLS is Arg-Lys-Lys-Arg-Arg-Gln-Arg-Arg-Arg (in one letter code, RKKRRQRRR) (SEQ ID NO: 3). This deletion is sufficient to abolish or significantly reduce nuclear localization of Tat or an AFP containing such a modified Tat domain. The loss of NLS activity can be measured, for example, by transiently transfecting cells with an AFP and assessing the AFP's subcellular localization using immunofluorescence. AFPs with an HIV tat domain that lacks NLS activity do not show the expected nuclear staining patterns. Such immunofluorescence methods are known to those of skill in the art and can use any antibody specific for the AFP or any domain of the AFP. Controls can be used when measuring activity but may not be necessary. Similarly, the integrin interaction activity of Tat depends on the presence of an RGD (arg-gly-asp) sequence in the protein (Barillari et al., (2002) Clin. Microbiol. Rev. 15:310-326). This sequence is not present in the HIV tat domains of the invention. Assays for measuring integrin binding are known in the art.

Transactivation activity of Tat is associated with Cys22 and Lys41 (Ruben et al., (1989) J. Virol. 63: 1-8). Accordingly, mutating these two amino acids can lead to loss of transactivation activity. For example, changing these two amino acids to glycine reduces or abolishes Tat's transactivation activity. A decrease or loss of transactivation activity can be measured, for example, using an HIV-1 LTR-chloramphenicol acetyltransferase (CAT) reporter in a standard CAT assay. Loss of CAT activity driven from the LTR promoter in the presence of the AFP (with a mutated Tat) when compared to CAT activity driven from the LTR promoter in the presence of a wild-type Tat demonstrates that the HIV tat domain of the AFP lacks transactivation activity (see, Example 2). In this regard, CAT assays are well-known in the art (e.g., Seed et al., (1988) Gene 67:271-277). If additional changes or other changes are needed to reduce or abolish transactivation activity, those changes can be introduced into the HIV tat domain of the AFP and tested in the manner described here and in Example 2. Any sequence alteration that abolishes transactivation activity in Tat is contemplated. In a preferred embodiment, the HIV tat domain comprises amino acids 1-92 of SEQ ID NO: 1 or a corresponding domain (as altered) from another HIV Clade consensus sequence.

Each HIV reverse transcriptase domain of the AFP lacks polymerase activity. Preferably, the AFP contains the entire RT with the protein divided in a manner to significantly decrease or abolish reverse transcriptase activity for each domain. One arrangement involves splitting RT into two domains. A preferred arrangement involves “swapping” or transposing of the N- and C-terminal halves of the protein, such that the sequences found at the C-terminus of the native protein are positioned closer to the N-terminus of the fusion protein than the sequences found at the N-terminus of the native RT protein. In some embodiments, one or more additional domains, such as a gp41 domain, the tat domain and the like, are interposed between two reverse transcriptase domain sequences. One way to reduce or abolish activity is to partition the protein such that the N-terminal portion and C-terminal portion are separated in the region encoding the active site of RT, so that neither half possesses polymerase activity. Two preferred HIV reverse transcriptase domains comprise (1) amino acids 1-271 or 1-272 (an amino terminal region) of RT from an HIV Clade A consensus sequence and (2) amino acids 273-450 (a carboxyl terminal region) of RT from an HIV Clade A consensus sequence, or a corresponding domain from another HIV clade consensus sequence.

The HIV nef domain of the AFPs is not myristylated, and preferably, includes at least about 50% to about 60% of the sequence of the native nef protein (“Nef”) to provide many CTL epitopes. For example, deletion of approximately 25% of the N-terminal portion of Nef prevents its myristylation and its ability to down-regulate CD4 and HLA class I molecules while retaining many of its CTL epitopes. In a preferred embodiment, the HIV nef domain comprises amino acids 65-206 of Nef from an HIV Clade A consensus sequence, or a corresponding domain from another HIV Clade consensus sequence.

The AFPs of the invention contain two CTL-rich domains from HIV gp41. These domains are also referred to herein as first and second CTL-rich HIV env domains. The first domain consists essentially of amino acids 699-742 of SEQ ID NO: 1 or the equivalent amino acids from HIV gp41 or an HIV gp41 consensus sequence. The second domain consists essentially of amino acids 743-843 of SEQ ID NO: 1 or the equivalent amino acids from HIV gp41 or an gp41 HIV consensus sequence. Amino acids 699-742 of SEQ ID NO: 1 correspond to amino acids 557-600 of the gp41 portion of gp160 from an HIV Clade A consensus sequence, and amino acids 743-843 of SEQ ID NO: 1 correspond to amino acids 765-856 of the gp41 portion of gp160 from an HIV Clade A consensus sequence.

As used herein, the term “human CTL epitope” refers to an epitope that is recognized and responded to by the CTLs of at least a portion of the human population. The human CTL epitopes included in the AFPs are associated with long-term non-progression to AIDS (Kaul; Rowland-Jones et al., (1998) J. Clin. Invest. 102: 1758-1765; Dorrell et al., (2000) AIDS 14: 1117-1122). A list of 14 human CTL epitopes associated with long-term non-progression, any of which are suitable for inclusion in the AFPs of the invention, is shown in Table 1. Of these, the epitopes that are derived from Clade A HIV proteins and restricted to HLA A*6802 are preferred. A preferred HLA A*6802 is DTVLEDINL (SEQ ID NO: 4). At least one, and preferably no more than six human CTL epitopes (over and above those present in the other HIV domains in the AFPs), is included in the AFPs of the invention. In general and preferably, the human CTL epitope(s) associated with long-term non-progression to AIDS are from the same Clade as the other HIV domains of the AFPs.

TABLE 1
CD8+ T-cell epitopes associated with
long-term non-progression to AIDS

| | HLA Class I | HIV-1 Protein | HIV | SEQ |
|---|---|---|---|---|
| Epitope | Restriction | of Origin | Clade | ID NO. |
| TPGPGVRYPL | B7 (*8101) | Nef | B | 5 |
| SPRTLNAWV | B7 (*8101) | p24 | B | 6 |
| DTVLEDINL | A*6802 | Pol | A | 4 |
| ETAYFILKL | A*6802 | Pol | A | 7 |
| SLYNTVATL | A2 | p17 | B | 8 |
| AIFQSSMTK | A33 | Pol | B | 9 |
| YPLTFGWCF | B18 | Nef | D | 10 |
| ALKHRAYEL | A2 | Nef | A/D | 11 |
| LSPRTLNAW | B57/58 | p24 | A | 12 |
| VSFEPIPIHY | A29 | gpl20 | B | 13 |
| KIRLRPGGK | A3 | p17 | B | 14 |
| DLNMMLNIV | B14 | p24 | A | 15 |
| DRFWKTLRA | B14 | p24 | B | 16 |
| ATPQDLNMML | B53 | p24 | A | 17 |

The AFPs of the invention can have additional, non-HIV domains to aid in characterization and monitoring of the AFP. Preferably such domains are at the N and/or C-termini of the AFP, but they can also be interposed between the HIV and human CTL domains of the AFP. For example, the additional domains can encode intra- or extracellular signals or sites that affect processing of the polypeptide (e.g., to include a protease cleavage site, signal sequence for intracellular localization or trafficking, or other such sequence), sites to aid protein purification and/or sites to aid protein localization. Sites useful for protein purification or localization include sequences that enable affinity binding. For example, epitopes recognized by antibodies (e.g., Pk, Flag, HA, myc, GST or H is) that are well known in the art can be included (Harlow et al., Using Antibodies: A Laboratory Manual, Cold Spring Harbor Laboratory Press, 1998). The additional domains can also be immunogenic in a laboratory animal (e.g., simian or murine CTL epitopes) and thereby provide an additional way to monitor the AFP during developmental research, preclinical studies and possibly during clinical use. When such additional immunogenic domains are used, the number of such domains should be minimized, preferably to no more than 3 or 4, to avoid interference with or competition for stimulation of HIV-specific immune responses.

In a preferred embodiment, the AFPs have a domain with at least one non-human CTL epitope that is recognized by the immune system of one or more laboratory animals, such as mice, non-human primates (including chimpanzees, rhesus macaques and other monkeys and the like), rabbits, rats, or other suitable laboratory animals. Inclusion of a non-human CTL epitope allows assessment of the quality, reproducibility, and/or stability of different batches of the AFPs using a laboratory test animal. Examples of such epitopes include the amino acid sequence STPESANL (SEQ ID NO: 18) which is a Mamu-A*01-restricted epitope from simian immunodeficiency virus (SIV) tat protein that is recognized by rhesus monkey CTLs and referred to here as “the SIV tat CTL epitope” (Allen et al., (2000b) Nature 407:386-390). Another example is SYIPSAEKI (SEQ ID NO: 19) which is a murine H-2 K^{d}-restricted CTL epitope from Plasmodium berghei and is also called the pb9 epitope (Romero et al., (1989) Nature 341: 323-326). Other suitable epitopes are known, e.g., the amino acid sequence ACTPYDINQML (SEQ ID NO: 20), which contains an epitope from SIV gag p27 recognized by rhesus macaque monkey CTLs (referred to herein as “the SIV gag p27 epitope”); and the sequence RGPGRAFVTI, a murine H-2 k-restricted CTL epitope from HIV gp41 protein which is also known as the P18-I10 epitope. Suitable non-human CTL epitopes are known or can be readily determined by those of skill in the art using techniques known for identifying CTL in laboratory animals.

The AFPs can also comprise a domain that is a small tag or marker to allow for detection of expression, localization, quantification of the amount of AFP and/or purification of the AFP. Suitable tags include, but are not limited to, epitopes recognized by mAbs, such as the epitope IPNPLLGLD (SEQ ID NO: 21) recognized by the Pk mAb (Hanke et al., (1992) J. Gen. Virol. 73:653-660); the epitope YPYDVPDYA (SEQ ID NO: 22) recognized by HA antibody; the epitope DYKDDDDK (SEQ ID NO: 23) recognized by Flag antibody; the epitope YTDIEMNRLGK (SEQ ID NO: 24) recognized by the VSV-G Tag antibody and the epitope EYMPME (SEQ ID NO: 25) recognized by the Glu-Glu antibody. Those of skill in the art can readily select suitable tags and markers for inclusion in an AFP.

The HIV domains and the human CTL epitopes of the AFPs can be contiguous within the protein. Alternatively, they can be separated by intervening amino acid sequences. The intervening amino acid sequences are generally non-HIV sequences, but can also comprise a small number of additional HIV amino acids. Intervening sequences, if present, range from 1-20 amino acids per intervening sequence domain and are preferably less than 10 amino acids, and even more preferably from 2-5 amino acids in length. For example, intervening sequences can be linkers, spacers or other sequences that optimize the expression levels of the AFPs. The intervening sequences can be used to optimize immunogenicity. Intervening sequences can also be added as a convenience to allow inclusion of useful restriction sites or to ensure that the domains of the AFPs are joined “in-frame” (e.g., for recombinantly-produced AFPs).

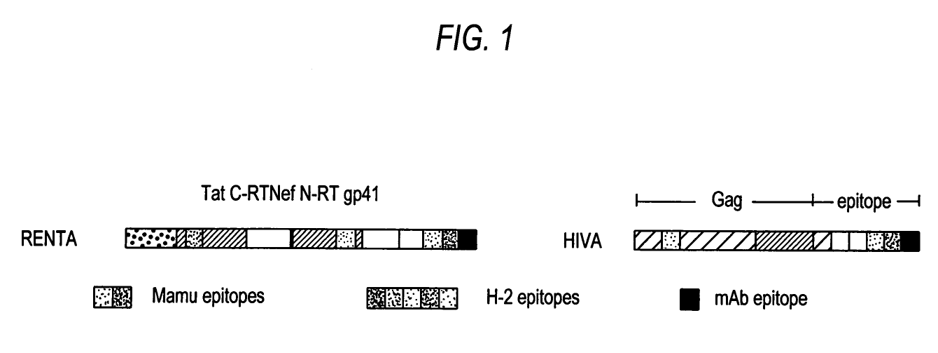

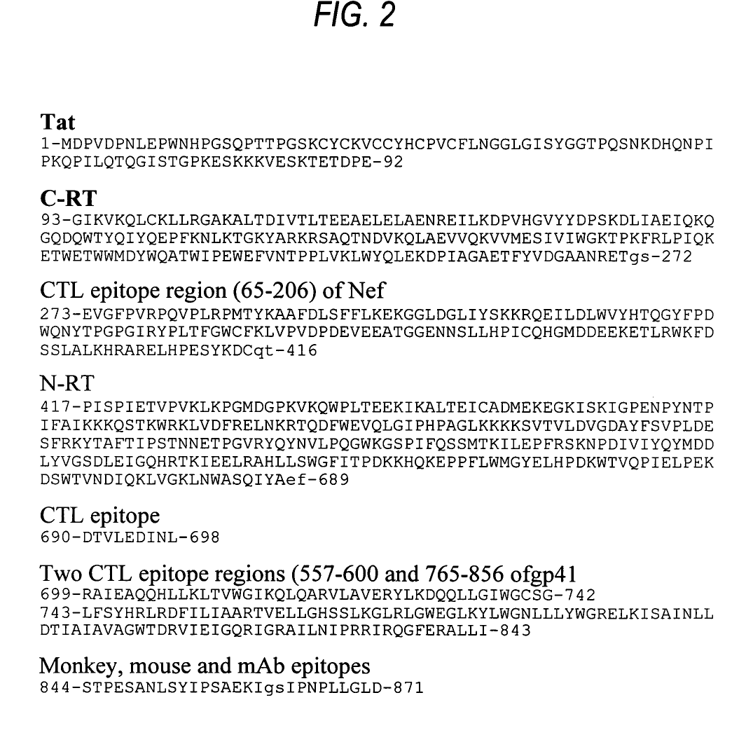

One example of an AFP of the invention is RENTA. RENTA is an AFP having 871 amino acids with 7 HIV domains and three additional domains. A schematic diagram of RENTA is shown in FIG. 1A and its amino acid sequence in FIG. 2. The RENTA protein, from amino to carboxyl terminus, comprises an HIV tat domain, a first HIV reverse transcriptase domain (the approximately carboxyl-terminal half), an HIV nef domain, a second HIV reverse transcriptase domain (the approximately amino-terminal half), a human HIV CTL epitope associated with long-term non-progression to AIDS, a first CTL-rich domain from gp41 (having amino acids 699-742 of SEQ ID NO: 1), a second CTL-rich domain from gp41 (having amino acids 743-843 of SEQ ID NO: 1), the SIV tat CTL epitope, the murine CTL epitope pb9, and the mAb epitope Pk. RENTA also contains intervening sequences. The correlation of domains and intervening sequences for the 871 amino acids of RENTA are shown in FIG. 2 (and in SEQ ID NO: 1) and as follows:
amino acids 1-92, the HIV tat domain;
amino acids 93-270, the first HIV reverse transcriptase domain (carboxyl-half);
amino acids 273-414, the HIV nef domain;
amino acids 417-687, the second HIV reverse transcriptase domain (amino-half);
amino acids 690-698, the human CTL epitope associated with long-term non-progression to AIDS;
amino acids 699-742, the first CTL-rich env domain;
amino acids 743-843, the second CTL-rich env domain;
amino acids 844-851, the SUV tat CTL epitope;
amino acids 852-860, the murine CTL epitope pb9;
amino acids 863-871, the mAb epitope Pk; and
amino acids 271-272, 415-416, 688-689 and 861-862, each pair being an intervening sequence.

Another aspect of the invention relates to nucleic acid molecules encoding AFPs of the invention. “Nucleic acid molecules” or “nucleic acid” as used herein means any deoxyribonucleic acid (DNA) or ribonucleic acid (RNA), including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex. The nucleic acid molecules of the invention have a nucleotide sequence that encodes the AFPs and can be designed to employ codons that are used in highly-expressed genes of the organism in which the AFP gene is expressed (or to be expressed). Typically, the nucleic acid has the entire coding sequence of the AFP as a single ORF, that is, without introns.

In a preferred embodiment, the codons encoding the AFP are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the AFPs in human cells. In other embodiments, for example, when the AFP is expressed in bacteria, yeast or other expression system, the codon usage pattern is altered to represent the codon bias for highly expressed genes in the organism in which the AFP is being expressed. Codon usage patterns are known in the literature for highly expressed genes of many species (e.g., Nakamura et al., (1996) Nucl. Acids Res. 24: 214-215; Wang et al, (1998) Mol. Biotechnol. 10: 103-106; McEwan et al. (1998) Biotechniques 24:131-136).

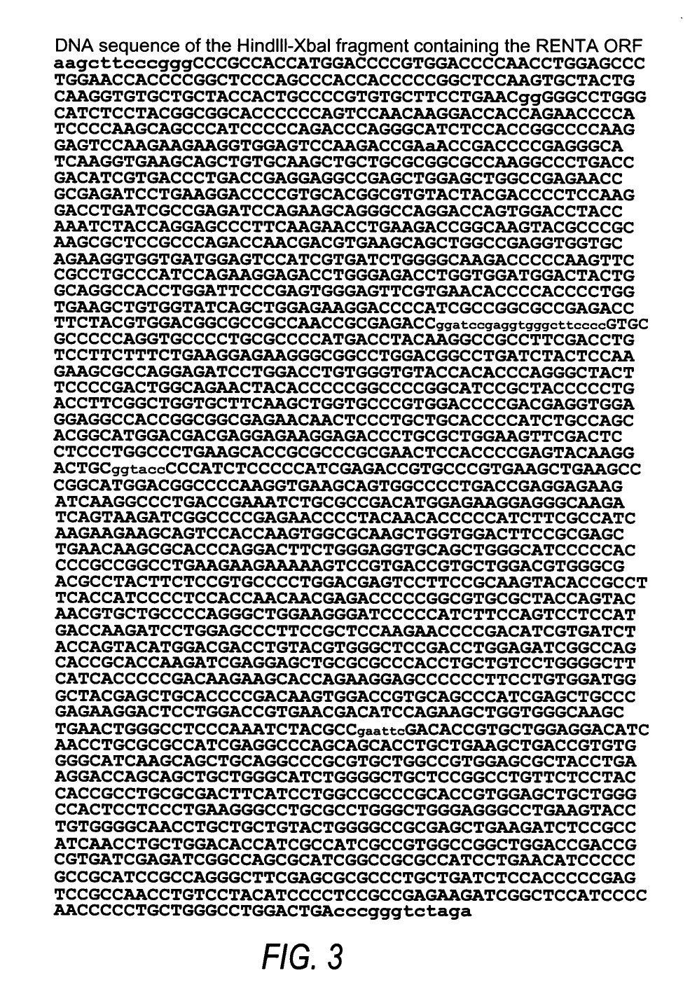

The nucleic acid sequence for RENTA is provided in FIG. 3. In one embodiment of the invention, the nucleic acid of the invention comprises the nucleotides encoding the RENTA coding sequence as shown in FIG. 3 (beginning at nucleotide 25 of SEQ ID NO: 2 and continuing to the stop codon). In another embodiment of the invention, the nucleic acid of the invention consists essentially of the sequence shown in FIG. 3.

Nucleic acid molecules encoding the AFPs of the invention can be incorporated into expression vectors and used to immunize subjects or used to express the protein in vitro, typically for protein production or for RNA production.

Expression vectors are well-known in the art and for the present invention share the common feature of having a protein coding sequence operably linked to regulatory control sequences that direct transcription and translation of the protein. Expression vectors are known and available for many organisms, including bacteria, fungi, yeast, animals (including mammals and particularly humans), birds, insects, plants and the like. Animals include, but are not limited to, mammals (humans, primates, etc.), commercial or farm animals (fish, chickens, cows, cattle, pigs, sheep, goats, turkeys, etc.), research animals (mice, rats, rabbits, etc.) and pets (dogs, cats, parakeets and other pet birds, fish, etc.).

Accordingly, expression vectors of the present invention have the coding sequence for an AFP of the invention operably linked to transcriptional and/or translational control sequences, depending on whether protein is being expressed or RNA is being produced. The expression vectors of the invention are useful to achieve expression of the AFP or a nucleic acid encoding the AFP in a particular host cell, including production of DNA or RNA encoding the AFP. Similarly, the expression vectors of the invention include plasmid, liposomal, microorganism and viral vectors useful to deliver the AFP (as protein or nucleic acid) to a host subject.

Expression vectors of the invention include plasmids, viral vectors, bacterial vectors, insect vectors, yeast vectors, mammalian cell vectors and the like. Whether the expression vector is capable of replication or self-amplification depends on the vector employed and the reason for its selection. Such characteristics can be readily determined by the skilled artisan when considering the requirements for expressing the AFP under the identified circumstances.

Expression vectors of the invention include those used for the expression of the AFPs in a laboratory animal, a mammal or, preferably, a human subject. These vectors are particularly useful for immunizing the animal, mammal or human subject to stimulate an immune response against the encoded AFP. Expression vectors useful in this regard include bacterial vectors, viral vectors, plasmids and liposomal formulations using nucleic acid (from plasmids or viruses). For bacterial vectors, the preferred vectors are attenuated to prevent proliferation of the bacterial carrier in the host or to only allowed self-limiting proliferation that will not lead to disease or other detrimental pathological effect. Killed bacteria are also useful. Viral vectors are preferably replication-defective, again to provide safety of use in the host. Plasmids, when used, can lack an origin of replication that functions in humans.

One example of a useful plasmid expression vector is the pTHr vector (Hanke 2000a) which controls expression using an enhancer/promoter/intron A cassette from the human cytomegalovirus immediate early protein and a bovine polyadenylation site. This plasmid uses a repressor-titration system for bacterial selection and does not carry any antibiotic-resistance genes (U.S. Pat. No. 5,972,708). Such a system lowers the total amount of DNA needed for delivery and increases the safety of the plasmid. Any plasmid vector safe for use in humans, mammals or laboratory animals is contemplated for use as well as any plasmid vector useful for protein purification from prokaryotic or eukaryotic expression systems.

Viral expression vectors are well known to those skilled in the art and include, for example, viruses such as adenoviruses, adeno-associated viruses (AAV), alphaviruses, retroviruses and poxviruses, including vaccinia viruses and particularly, the modified vaccinia Ankara virus (MVA; ATCC Accession No. VR-1566). Such viruses, when used as expression vectors are innately non-pathogenic in the selected host humans or have been modified to render them non-pathogenic in the selected host. For example, replication-defective adenoviruses and alphaviruses are well known and can be used as gene delivery vectors. A preferred viral vector is MVA, which is a highly attenuated vaccinia strain which fails to replicate in most mammalian cells (Mayr et al., (1975) Infection 105:6-14). AFPs can be cloned into many sites of the MVA and used to immunize a subject, especially a human subject, and generate an HIV-specific immune response against the encoded AFP. Useful MVA cloning sites, for example include the thymidine kinase and deletion III loci (Chakrabarti et al., (1985) Mol. Cell. Biol. 5: 3403-3409; Meyer, H. et al (1991) J. Gen. Virol. 72: 1031-8; Altenburger, W. et al (1989) Arch. Virol. 105(1-2): 15-27).

Other viral vectors useful for delivering the AFPs include alphavirus vectors, particularly those based on the replicons of Semliki Forest Virus (SFV), Sindbis virus and Venezuelan Equine Encephalitis virus (VEE) (see, e.g., Smerdou et al., (2000) Gene. Ther. Regul. 1:33-63; Lundstrom et al., (2002) Technol. Cancer Res. Treat. 1: 83-88; Hanke 2003). Alphavirus replicons are useful expression vectors and can refer to RNA or DNA comprising those portions of the alphavirus genomic RNA essential for transcription and export of a primary RNA transcript from the cell nucleus to the cytoplasm, for cytoplasmic amplification of the transported RNA and for RNA expression of a heterologous nucleic acid sequence. Thus, the replicon encodes and expresses those non-structural proteins needed for cytoplasmic amplification of the alphavirus RNA and expression of the subgenomic RNA, as well as an AFP of the invention. It is further preferable that the alphavirus replicon cannot be encapsidated to produce alphavirus particles or virions. This can be achieved by replicons, which lack one or more of the alphavirus structural genes, and preferably all of the structural genes, such as occurs with a one-helper or two-helper alphavirus vector system. In a preferred embodiment, alphavirus replicons are capable of being transcribed from a eukaryotic expression cassette and processed into RNA molecules with authentic alphavirus-like 5′ and 3′ ends.

Alphavirus replicons and expression vectors containing them are well known in the art and many vectors containing a wide range of alphavirus replicons have been described. Examples of such replicons can be found, e.g., in U.S. Pat. Nos. 5,739,026; 5,766,602; 5,789,245; 5,792,462; 5,814,482; 5,843,723; and 6,531,313; and in Polo et al., (1998) Nature Biotechnol. 16: 517-518 and Berglund et al., (1998) Nature Biotechnol. 16: 562-565. Alphavirus replicons can be prepared from any alphavirus or any mixture of alphavirus nucleic acid sequences. In this regard, the preferred alphavirus replicons are derived from Sindbis virus, SFV, VEE or Ross River virus.

Other viral expression vectors include flaviviruses (WO02/072835), such as yellow fever virus, Dengue virus and Japanese encephalitis virus, poxviruses such as vaccinia virus (U.S. Pat. No. 5,505,941), avipoxviruses such as fowlpox virus (Kent;) and canary pox virus (Clements-Mann et al., (1998) J. Infect. Dis. 177: 1230-1246; Egan et al., (1995) J. Infect. Dis. 171: 1623-1627; U.S. Pat. No. 6,340,462), including attenuated avipoxviruses such as TROVAC (U.S. Pat. No. 5,766,599) and ALVAC (U.S. Pat. No. 7,756,103), picornaviruses such as poliovirus (U.S. Pat. Nos. 6,780,618; 6,255,104; WO92/014489) and rhinovirus, herpesviruses (WO87/000862; WO 87/04463; WO97/014808) such as Varicella zoster virus (VZV; WO97/004804), NYVAC (New York vaccinia virus with 18 gene deletions selected to decrease pathogenicity) (Hel et al., (2001) J. Immunol. 167: 7180-7191; U.S. Pat. Nos. 5,494,807; 5,762,938; 5,364,773); Adenovirus (AdV; WO95/02697; WO95/11984; WO95/27071; WO95/34671), adeno-associated virus (AAV; U.S. Pat. Nos. 4,797,368; 5,474,935), influenza virus (WO03/068923; WO02/008434; WO00/053786), cauliflower mosaic virus (U.S. Pat. No. 4,407,956), tobacco mosaic virus (TMV)(Palmer et al, (1999) Arch. Virol. 144: 1345-1360; WO93/003161) and NS1 tubules of bluetongue virus (Adler et al., (1998) Med. Microbial. Immunol. (Berl) 187: 91-96). Many of these vectors are readily available and conditions applicable for their use are well-known to the skilled artisan.

Expression vectors of the invention also include bacterial expression vectors for administration to a laboratory animal, mammal or human subject. Such bacterial expression vectors (attenuated, invasive bacteria) are bacteria that contain a plasmid or an expression cassette encoding an AFP of the invention. The expression cassette can drive expression in the bacteria or in eukaryotic cells. In the former, expression is achieved before introducing the bacterial cells into the host, whereas in the latter, expression occurs in the host and can be driven by the host cellular machinery. U.S. Pat. Nos. 5,877,159; 6,150,170; 6,500,419 and 6,531,313 describe bacterial vectors that invade animal cells without establishing a productive infection or causing disease and thus permit the introduction of a expression cassette encoding an AFP into a eukaryotic cell to obtain expression of the AFP.

Suitable bacterial expression vectors include Mycobacterium bovis, Bacillus Calmette Guerin (BCG), and attenuated strains of Salmonella (especially the “double aro” mutants of Salmonella that are being developed as vaccines for diarrheal diseases), Shigella (see Shata et al., (2000) Mol. Med. Today 6: 66-71), Neisseria and Listeria monocytogenes. Preferred Salmonella typhi strains include CVD908Δasd, CVD908ΔhtraA and CVD915. The CVD908Δasd Salmonella strain derives from CVD908 (Tacket et al., (1992) Vaccine 10: 443-446) by deletion of the asd gene that encodes the aspartate b-semialdehyde dehydrogenase (asd), an enzyme necessary for the synthesis of diaminopimelic acid (DAP) from aspartate. CVD908ΔhtrA is a S. typhi strain with the htrA gene deleted. This mutation knocks out a heat shock gene that further attenuates the strain (Tacket et al., (1997) Infect. Immunol. 65:452-456). CVD915 is an attenuated S. typhi strain that has a deletion of the guaBA locus, resulting in its attenuation (Pasetti et al., Clin. Immunol. 92:76-89, 1999). This strain has been shown to be excellent for the delivery of DNA vaccines in animal studies and is entering Phase I trials. A preferred Shigella strain is S. flexneri CVD 1207. This strain has deletions of the sen, set, virG and guaBA genes that renders it well attenuated while preserving its immunogenicity (Kotloff et al., Infect. Immunol. 68:1034-1039, 2000).

The control sequences, such as promoters and the like, in the expression vectors are often heterologous with respect to the host. The expression of the AFP nucleotide sequence in the expression vector can thus be under the control of a constitutive promoter or of an inducible promoter, which initiates transcription only when the host cell is exposed to some particular external stimulus. In the case of a multicellular organism, such as an animal, the promoter can also be specific to a particular tissue or organ.

Expression vectors of the invention are also used for preparation and purification of the AFPs of the invention. Vectors in this regard are typically used in bacteria, yeast, insect or mammalian cells. The regulatory sequences directing expression of the nucleic acid molecule encoding the AFP are chosen based on the host cell (e.g., bacterial, yeast, insect or mammalian cells) from which the expression is being directed. Appropriate regulatory sequences for a particular host cell and expression vector are well known. The expression vectors containing the AFP can be introduced into these cells by well-known methods in the art, which depend, inter alia, on the type of cell and whether the duration of expression is transient or stable. For example, calcium chloride transfection is commonly utilized for prokaryotic cells, whereas calcium phosphate treatment, lipofection or electroporation is used for many eukaryotic cells. Any transfection, infection, transformation or suitable technique for introducing an expression vector into a cell, whether prokaryotic or eukaryotic, known to the skilled artisan can be used.

There are numerous Escherichia coli vectors and cells known to one of ordinary skill in the art that are useful for expression of the AFPs of the invention. Other microbial hosts suitable for use include bacilli, such as Bacillus subtilis, and other enterobacteria, such as Salmonella, Serratia, as well as various Pseudomonas species. These prokaryotic hosts can support expression vectors, which typically contain expression control sequences operable primarily in the host cell. Any number of a variety of well-known promoters can be present, such as the lactose promoter system, a tryptophan (Trp) promoter system, a β-lactamase promoter system, or a promoter system from phage λ. The promoters will typically control expression, optionally with an operator sequence and have ribosome binding site sequences for example, for initiating and completing transcription and translation. If necessary, an amino-terminal methionine can be provided by insertion of a Met codon 5′ and in-frame with the protein. Among vectors preferred for use in bacteria include pQE70, pQE60 and pQE9, available from QIAGEN, Inc.; pBluescript vectors, Phagescript™ vectors, pNH8A, pNH16a, pNF118A, pNH46A, available from Stratagene Cloning Systems, Inc.; and ptrc99a, pKK223-3, pKK233-3, pDR540, pRIT5 available from Pharmacia Biotech, Inc. Other expression vector systems are based on β-galactosidase (p-gal; pEX), maltose binding protein (pMAL) and glutathione S-transferase (pGST) (see e.g., Smith, (1988) Gene 67: 31-40 and Abath. (1990) Peptide Research 3: 167-168).

Yeast cells can also be used to direct expression of the AFPs of the invention. There are several advantages to yeast expression systems that make use of the yeast system desirable in certain circumstances, including providing disulfide pairing, post-translational modifications, protein secretion and easy isolation when protease cleavage site is inserted upstream of from the AFP coding sequence. The Saccharomyces cerevisiae pre-pro-α-factor leader region (encoded by the MFa-I gene) is routinely used to direct protein secretion from yeast (Brake et al., (1984) Proc. Natl. Acad. Sci. USA 82: 4642-4646; U.S. Pat. No. 4,870,008). The leader region of pre-pro-α-factor contains a signal peptide and a pro-segment, which includes a recognition sequence for a yeastprotease encoded by the KEX2 gene. This enzyme cleaves the precursor protein on the carboxyl side of a Lys-Arg dipeptide cleavage-signal sequence. The AFP coding sequence can be fused in-frame to the pre-pro-α-factor leader region. This construct can then be put under the control of a strong transcription promoter, such as the alcohol dehydrogenase I promoter or a glycolytic promoter. The fusion protein coding sequence can be followed by a translation termination codon, which can be followed by transcription termination signals. Vectors useful for expression in yeast include, without limitation, the 2μ circle plasmid (Broach, J. R. et al, (1979) Gene 8(1): 121-33).

Efficient post-translational modification and expression of recombinant proteins can also be achieved in Baculovirus systems in insect cells (“Baculovirus Expression Protocols,” Humana Press Inc.; WO92/005264). These systems are well known in the art.

Mammalian cells are useful to express and purify the AFPs of the invention, especially when the protein is purified for administration to mammalian subjects. Vectors useful for the expression of proteins in mammalian cells often have strong viral promoters to direct expression and can also include other sequences that are useful for directing expression in human cells, such as enhancers, polyadenylation signals, and other signal sequences for promoting transcription, translation, i.e., internal ribosomal entry sites (IRES), and/or the processing of the AFPs of the invention. Alternatively or additionally, the plasmid in the DNA vaccine or immunogenic composition can further contain and express in an animal host cell a nucleotide sequence encoding a heterologous tPA signal sequence such as human tPA and/or a stabilizing intron, such as intron II of the rabbit β-globin gene.

Depending on the vector, selectable markers encoding antibiotic resistance may be present when used for in vitro purification, such as, but not limited to, ampicillin, neomycin, zeocin, kanamycin, bleomycin, hygromycin, chloramphenicol, among others. Selection systems that do not use antibiotic resistance genes can also be used in the expression vector and mammalian host system. Promoter sequences that can be used to direct expression of the AFPs include, but are not limited to, strong viral promoters, such as the promoter from human cytomegalovirus (CMV), the promoter from the thymidine kinase gene of herpes simplex virus (HSV), promoters from adenoviruses and composite promoters such as the EF-1a/HTLV promoter (InVitrogen) and the ferritin composite promoters comprised of the FerH or FerL core promoters (InVitrogen) among others. Among preferred eukaryotic expression vectors are pWLNEO, pSV2CAT, pOG44, pXT1 and pSG available from Stratagene; and pSVK3, pBPV, pMSG and pSVL available from Pharmacia. The AFP coding sequence can be introduced into a mammalian cell line capable of synthesizing intact proteins have been developed in the art and include, but are not limited to, CHO, COS, 293, 293T, HeLa, NIH 3T3, Jurkat, myeloma and PER.C6 cell lines. Presence of the expression vector-derived RNA in the transfected cells can be confirmed by Northern blot analysis and production of a cDNA or opposite strand RNA corresponding to the protein coding sequence can be confirmed by Southern and Northern blot analysis, respectively.

Cell transformation techniques and gene delivery methods (such as those for in vivo use to deliver genes) are well known in the art. Any such technique can be used to deliver a nucleic acid or expression vector encoding an AFP of the invention to a cell or subject, respectively.

The AFPs of the invention can be purified from bacterial, yeast, insect or mammalian cells using techniques well-known in the art. For example, the AFPs can be purified or concentrated using ammonium sulfate or ethanol precipitation, acid extraction, anion or cation exchange chromatography, phosphocellulose chromatography, hydrophobic interaction chromatography, affinity chromatography, immuno-affinity chromatography, hydroxyapatite chromatography, lectin chromatography, molecular sieve chromatography, isoelectric focusing, gel electrophoresis, combinations of these methods using monitoring techniques to follow the distribution of the AFP at each purification step as well as the purity of the AFP. Some or all of the foregoing purification steps, in various combinations or with other known methods, can also be employed to provide substantially purified, isolated AFPs of the invention. If the AFP contains an epitope recognized by a monoclonal or polyclonal antibody, then immunoaffinity purification can be used alone or in conjunction with the above techniques. For immunoaffinity chromatography, the AFP (or a cellular extract or other mixture containing the AFP) can be purified by passage through a column containing a resin, which has bound thereto antibodies specific for the antigenic peptide. Immunoaffinity purification can also be conducted in batches when the affinity reagent is bound to a solid support. Such techniques are well known in the art.

In yet another aspect, the invention provides an immunogenic composition comprising the AFPs, nucleic acids or expression vectors of the invention in admixture with an pharmaceutically acceptable carrier. Such carriers are also acceptable for immunological use. The immunogenic compositions of the invention are useful to stimulate an immune response against HIV as one or more components of a prophylactic or therapeutic vaccine against HIV for the prevention, amelioration or treatment of AIDS.

The compositions of the invention may be injectable liquid solutions or emulsions. To prepare such a composition, an AFP, nucleic acid or expression vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants include, but are not limited to mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC31; see Schellack, C. et al (2003) Proceedings of the 34^{th} Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169)7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara(D; U.S. Pat. Nos. 4,689,338; 5,238,944), and the CCR5 inhibitor CMPD167 (see Veazey, R. S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H.R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IGF-1, IFN-α, IFN-β, and IFN-γ (Boyer et al., (2002) J. Liposome Res. 121:137-142; WOO/095919), immunoregulatory proteins such as CD40L (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, on the same expression vectors as those encoding the AFP of the invention or on separate expression vectors.

The immunogenic compositions can be designed to introduce the AFP, nucleic acid or expression vector to a desired site of action and release it at an appropriate and controllable rate. Methods of preparing controlled-release formulation are known in the art. For example, controlled release preparations can be produced by the use of polymers to complex or absorb the immunogen and/or immunogenic composition. A controlled-release formulations can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulssions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the AFP, nucleic acids and expression vectors of the invention (collectively, the immunogens) in the immunogneic composition of the invention can he readily determined by those of skill in the art. For example, the dosage of the imnmnogens can vary depending on the route of administration and the size of the host. A suitable dose of AFP of the invention can range from about 1-10 μg to about 5000 mg, and is typically from about 500 pg to about 100 mg, depending inter alia on the molecular weight of the AFP, the route of delivery, the delivery means and the body mass of the recipient. A suitable dose of nucleic acid of the invention can range from about 1 μg to about 100 mg, and more typically from about 10-100 μg to about 1-10 mg again depending, inter alia, on the factors assessed for protein delivery, as well as the size of the nucleic acid molecule. The dosages for delivery of expression vectors of the invention depends additionally on the nature of the expression vector. When the vector is an RNA or DNA molecule (including plasmids or a plasmid incorporated in a lipid or other delivery particle), then the amount of expression vector in a dosage is similar to that of the nucleic acids of the invention. The dosage for bacterial expression vectors is conveniently characterized according to colony forming units (cfu). The dose will preferably range from about 10⁴ to about 10¹⁰ cfu and more preferably from about 10⁶ to about 10¹⁰ cfu, as well as from about 10⁸ to about 10⁹ cfu. The dosage for viral expression vectors depends on the nature of the vector, e.g., whether the vector is an alphavirus, an adenovirus, AAV, a vaccinia virus, a retrovirus and the like. Any of these doses can be calculated on a unit dosage basis or as an amount per kilogram body weight.

Doses for administering viral vectors are well known and can be determined by those of skill in the art if needed. By way of example, when the agent is a viral vector such as a replication-defective adenovirus, the dosage can range from about 10⁶ to about 10¹² plaque forming units (pfu), and is preferably between about 10⁸ to about 10¹⁰ pfu. For stable and efficient transduction using a recombinant AAV, the dosage can be from about 1×10⁵ WU (infectious units) of AAV per gram body weight to about 1×10⁹ IU AAV per gram body weight, and preferably from about 1×10⁶ IU AAV per gram body weight to about 1×10⁷ IU AAV per gram body weight. For vaccinia and MVA, dosages ranging from about 10⁵ to about 10¹⁰ pfu, are useful; dosages of about 10⁷ to about 10⁸ pfu are often used.

Other suitable doses can be determined by those of skill in the art. To determine appropriate doses, those of skill in the art can measure the immune response of subjects by conventional immunological techniques and adjust the dosages as appropriate. Such techniques include but are not limited to, e.g., chromium release assay, tetramer binding assays, IFN-γ ELISPOT assays and intracellular cytokine assays as well as other immunological detection assays, e.g., as detailed in Harlow.

The present invention provides methods for expressing an AFP of the invention in animal cells by introducing an expression vector of the invention into the animal cells and culturing those cells under conditions sufficient to express said AFP. The expression vector can be introduced by any appropriate method including, but not limited to, transfection, transformation, infection, electroporation, particle bombardment and the like. Such techniques are standard in the art. After introducing the expression vector, the cells are maintained under the appropriate culture conditions (i.e., for a time and at the appropriate conditions) to maintain cell viability at least until the AFP is expressed. In some instances, for example with alphavirus replicon vectors, expression of the AFP includes production of an RNA molecule encoding the AFP.

In addition, the invention provides methods for introducing and expressing an AFP of the invention in an animal by delivering an expression vector of the invention in to the animal and thereby obtaining expression of the AFP in the animal. Any delivery method can be used including intramuscular, intravenous, intradermal, mucosal, topical or other delivery method, such as the particle bombardment method by Powderject (a needle-less delivery system to the skin that is actuated by helium gas). Such techniques are well known to those of skill in the art. The expression vectors can be formulated as needed to improve stability and delivery efficiency. Once the expression vector is delivered, the ORF of the AFP is transcribed (if needed) and translated to express the encoded AFP.

Such methods for expressing AFPs in animal cells and in animals are useful, for example, as clinical or other research tools for studying the mechanisms of AFP expression, localization of AFPs and the effects of various control elements on AFP expression and localization.

In accordance with the invention, the AFPs, nucleic acids and expression vectors of the invention can serve as immunogens for inducing immune responses in animals, particularly HIV-specific CTL immune responses. Hence as used herein, the immunogen is the molecule that is delivered to the animal and that directly or indirectly leads to production of an immune response (either humoral or cellular). An HIV immunogen stimulates a response against HIV which response can be cellular or humoral. RENTA and HIVA are examples of HIV protein immunogens. pTHr.RENTA and pTHr.HIVA are examples of DNA- or plasmid-vectored HIV immunogens. MVA.RENTA and MVA.HIVA are examples of virally-vectored HIV immunogens.

The present methods are useful as research tools when immunizing laboratory animals to study the immune response to these immunogens either alone or in conjunction with other HIV immunogens, as well as with or without adjuvants. More particularly, the methods can be for prophylactic or therapeutic prevention, amelioration or treatment of HIV in humans. When provided prophylactically, the methods are ideally administered to a subject in advance of any evidence of HIV infection or in advance of any symptom due to AIDS, especially in high-risk subjects. The prophylactic administration of the immunogens can serve to prevent or attenuate AIDS in a human subject. When provided therapeutically, the methods can serve to ameliorate and treat AIDS symptoms and are advantageously used as soon after infection as possible, preferably before appearance of any symptoms of AIDS but may also be used at (or after) the onset of the disease symptoms.

The recombinant vectors express a nucleic acid molecule encoding AFPs of the present invention. In particular, the AFPs can be isolated, characterized and inserted into vector recombinants. The resulting recombinant vector is used to immunize or inoculate an animal. Expression in the subject of the AFPs, can result in an immune response in the animal to the expression products of the AFP. Thus, the recombinant vectors of the present invention may be used in an immunological composition or vaccine to provide a means to induce an immune response, which may, but need not be, protective.

To induce or stimulate an immune response, an AFP or an expression vector of the invention or AFP of the invention is delivered one or more times into the animal so that the encoded AFP is expressed at a level sufficient to stimulate an immune response to the AFP, or the AFP is provided in an amount sufficient to stimulate an immune response to AFP. Any delivery method can be used including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradermal injection of DNA using “gene gun” technology (Johnston et al., (1994) Meth. Cell Biol. 43:353-365). Alternatively, delivery routes (especially for bacterial expression vectors, e.g., attenuated Salmonella or Shigella spp.) can be oral, intranasal or by any other suitable route. Delivery also be accomplished via a mucosal surface such as the anal, vaginal or oral mucosa.

Immunization schedules (or regimens) are well known for animals (including humans) and can be readily determined for the particular animal and immunogen (whether an AFP or an expression vector). Hence, the immunogens can be administered one or more times to the animal. Preferably, there is a set time interval between administration of the immunogen. While this interval varies for every animal, typically it ranges from 10 days to several weeks, and is often 2, 4, 6 or 8 weeks. For humans, the interval is typically from 2 to 6 weeks. The immunization regimes typically have from 1 to 6 administrations of immunogen, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods include a variety of prime-boost regimens, especially DNA prime-MVA boost regimens. In these methods, one or more priming immunizations are followed by one or more boosting immunizations. The actual antigen can be the same or different for each immunization and the type of immunogen (e.g., protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

A specific embodiment of the invention provides methods of stimulating an immune response against HIV in a human by administering an AFP of the invention, a nucleic acid of the invention and/or an expression vector of the invention one or more times to a subject wherein the AFP is administered in an amount or expressed at a level sufficient to stimulate an HIV-specific CTL immune response in said subject. Such immunizations can be repeated multiple times at time intervals of at least 2, 4 or 6 weeks (or more) in accordance with a desired immunization regime. The method can be used in combination with other HIV immunogens, including proteins or expression vectors that encode such other antigens. When used in combination, the other HIV immunogens can be administered at the same time or at different times as part of an overall immunization regime, e.g., as part of a prime-boost regimen or other immunization protocol. Many other HIV immunogens are known in the art, one such preferred immunogen is HIVA (described in WO 01/47955), which can be administered as a protein, on a plasmid (e.g., pTHr.HIVA) or in a viral vector (e.g., MVA.HIVA). A schematic representation of HIVA is shown in FIG. 1B.

For example, one method of stimulating an immune response against HIV in a human subject comprises administering at least one priming dose of an HIV immunogen and at least one boosting dose of an HIV immunogen, wherein the immunogen in each dose can be the same or different, provided that at least one of the immunogens is an AFP of the invention, a nucleic acid encoding an AFP of the invention or an expression vector encoding an AFP of the invention, and wherein the immunogens are administered in an amount or expressed at a level sufficient to stimulate an HIV-specific immune response in the subject. The HIV-specific immune response can include an HIV-specific CTL immune response. Such immunizations can be done at intervals, preferably of at least 2-6 weeks.

In accordance with this method, pTHr.RENTA is administered one or more times as the priming dose or MVA.RENTA is administered one or more times as the boosting dose, with or without the priming dose having been pTHr.RENTA. As an example of using another HIV immunogen in this method, the priming dose can be pTHr.HIVA and the boosting dose can be MVA.RENTA or a mixture of MVA.RENTA and MVA.HIVA. Alternatively, the priming dose can be pTHr.RENTA and the boosting dose can be MVA.HIVA or a mixture of MVA.RENTA and MVA.HIVA. Further combinations are possible, e.g., use of pTHr.RENTA as the priming dose followed by MVA.HIVA or a mixture of MVA.RENTA and MVA.HIVA as the boosting dose, or the use of a mixture of pTHr.HIVA and pTHr.RENTA as the priming dose followed by MVA.HIVA, MVA.RENTA or a mixture of MVA.RENTA and MVA.HIVA as the boosting doses. When mixtures are used in the priming or boosting doses, the components can be mixed together for administration or administered separately. When administered separately, the components can be also be administered sequentially as multiple separate priming or boosting doses done at an interval of 2-6 weeks from each other. One example of an immunization regimen of this method is to administer two priming doses at weeks 0 and 4, each dose being a mixture of pTHr.HIVA and pTHr.RENTA, followed by administration of two boosting doses at weeks 8 and 12, each dose being a mixture of MVA.RENTA and MVA.HIVA.

The immune response induced by the methods of the invention can be assessed by standard techniques known in the art. For CTL responses, such techniques include but are not limited to, intracellular IFN-γ staining assays, tetramer assays, ELISPOT assays, and ⁵¹Cr-release assays. Other immune responses can be assessed as described in Harlow.

The present invention also comprehends compositions and methods for making and using vectors, including methods for producing gene products and/or immunological products and/or antibodies in vivo and/or in vitro and/or ex vivo (e.g., the latter two being, for instance, after isolation therefrom from cells from a host that has had a non-invasive administration according to the invention, e.g., after optional expansion of such cells), and uses for such gene and/or immunological products and/or antibodies, especially neutralizing antibodies to HIV (reviewed in Haigwood, N. L. and Stamatatos, L. (2003) 17 (Suppl 4: S67-71), including in diagnostics, assays, therapies, treatments, and the like. The resulting neutralizing antibodies can be used separately, or in combination with the AFPs of the present invention to enhance or modulate immunogenic or immunological responses to HIV, SIV, or SIV/HIV hybrids. The neutralizing antibodies can be tailored for specificity to a particular clade or circulating recombinant form.

The invention also includes the use of the vectors expressing AFPs in the research setting. The vectors can be used to transfect or infect cells or cell lines of interest to study, for example, cellular responses to gene products expressed from the heterologous sequences of interest, or signal transduction pathways mediated by proteins encoded by the heterologous sequences of interest.

In the research setting, it is often advantageous to design recombinant vectors or viruses that comprise reporter genes that can be easily detected by laboratory assays and techniques. Reporter genes are well known in the art and can comprise resistance genes to antibiotics such as, but not limited to, ampicillin, neomycin, zeocin, kanamycin, bleomycin, hygromycin, chloramphenicol, among others. Reporter genes can also comprise green fluorescent protein, the lacZ gene (which encodes β-galactosidase), luciferase, and β-glucuronidase.

The invention further relates to the product of expression of the AFP and uses thereof, such as to produce a protein in vitro, or to form antigenic, immunological or vaccine compositions for treatment, prevention, diagnosis or testing; and, to DNA from the recombinant vectors, which are useful in constructing DNA probes, antisense RNA molecules, small interfering RNA molecules (siRNA), ribozymes, and PCR primers.

The AFPs of the present invention can also be altered or modified to include sequences from SIV, or from SIV/HIV hybrids, to produce an therapeutic or prophylactic immunogenic or immunological response in non-human primates. One of the skill in the art can easily modify the AFPs of the present invention to encompass SIV sequences and CTL epitopes to induce an immune response that may, but need not be, protective.

It is to be understood and expected that variations in the principles of invention herein disclosed in exemplary embodiments may be made by one skilled in the art and it is intended that such modifications, changes, and substitutions are to be included within the scope of the present invention. All of the patents and publications cited herein are hereby incorporated by reference.

The following examples are given for the purpose of illustrating various embodiments of the invention and are not meant to limit the present invention in any fashion.

### EXAMPLES

### Example 1

### RENTA: Plasmid and MVA Construction

The RENTA gene fragment is approximately 2.6 kb and was made synthetically using HIV-1 Clade A consensus sequence for each HIV protein domain and preferred human amino acid codon usage (Andre). The RENTA ORF is preceded by a consensus Kozak sequence to −12 nucleotides (Kozak, (1987) Nucleic Acid Res. 15:8125-8148). The RENTA ORF is incorporated in a DNA expression vector, pTHr, and in a viral expression vector, modified virus Ankara (MVA). All recombinant DNA manipulations used standard procedures (Sambrook et al., Molecular Cloning; A Laboratory Manual (2nd ed.), Cold Spring Harbor Press, Cold Spring Harbor, N.Y. 1989).

pTHr.RENTA Construction: A synthetically-constructed HindIII-XbaI fragment of 2,646 by carries the RENTA ORF. This fragment has the overall structure of HindIII-SmaI-HIV tat domain-HIV C-terminal reverse transcriptase domain-BamHI-HIV nef domain-KpnI-HIV N-terminal reverse transcriptase domain-EcoRI-human CTL epitope-first HIV env domain-second HIV env domain-monkey, mouse and mAb epitopes-SmaI-XbaI. Each of the sections flanked by restriction endonuclease sites was constructed separately from partially overlapping, approximately 90-mer oligonucleotides and sequenced to verify accuracy. When a sequence error was detected, the improper nucleotide(s) was replaced with the correct nucleotide using site-directed mutagenesis. The four sections were sequentially assembled into plasmid pTH (Hanke 1998a) and as the last cloning step, the β-lactamase gene from was removed therefrom by cutting the plasmid at the BspHI sites and religating the linear fragment containing RENTA. The resulting plasmid is the pTHr.RENTA expression vector. The pTHr.RENTA plasmid uses an auxotroph repressor-titration system for bacterial selection and does not carry any antibiotic-resistance gene (Williams et al., (1998) Nucleic Acid Res. 26:2120-2124). In the pTHr vector, RENTA transcription is controlled by an efficient enhancer/promoter/intron A cassette derived from the human cytomegalovirus strain AD 169 (Whittle et al., (1987) Protein Eng. 1: 499-505) and a bovine polyadenylation site (Goodwin et al., (1992) J. Biol. Chem. 267: 16330-16334). Preparation of MVA-RENTA: the RENTA Fragment was Cut Out of pTHr.RENTA Using XmaI and ligated into the XmaI site of transfer vector pSC11 (Chakrabarti) to produce the vector pSC 11.RENTA used in the preparation of recombinant MVA.RENTA. The plasmid pSC11.RENTA carries the β-galactosidase gene.

The RENTA-coding fragment was inserted into the thymidine kinase locus of the virus genome under the P7.5 early/late promoter using plasmid pSC 11, which co-delivered a β-galactosidase gene to facilitate screening, titration and stability studies of the recombinant MVA-RENTA (Chakrabarti). This marker enzyme is commonly expressed by human enteric bacteria and has been safe in several clinical trials including healthy HIV-uninfected volunteers vaccinated with MVA.HIVA.

Briefly, recombinant MVA.RENTA virions were produced from chicken embryo fibroblasts (CEF) cells grown in Dulbeco's Modified Eagle's Medium supplemented with 10% fetal calf serum (FCS), penicillin/streptomycin and glutamine (DMEM 10) that had been infected with parental MVA at a multiplicity of infection (MOI) of 1 and transfected using Superfectin (Qiagen, Germany) with 3 μg of endotoxin-free pSC 11.RENTA. Recombinants were identified by a blue color reaction in the presence of X-gal. Recombinants were subjected to five rounds of plaque purification, after which a master virus stock was grown, purified on a 36% sucrose cushion, titered and stored at −80° C. until use. The presence of the correct RENTA ORF was confirmed by sequencing and immunofluorescent detection of the protein in MVA.RENTA-infected cells.

Preparation of pIRES2-RENTA-EGFP: The RENTA fragment was cut out of pTHr.RENTA using XmaI and ligated into the XmaI site of vector pIRES2-EGFP (Clontech, USA) for the preparation of vector pIRES2-RENTA-EGFP. The parent vector expresses enhanced green fluorescent protein (EGFP), which was used in the assays demonstrating inactivation of Nef functions

### Example 2

### RENTA Expression in Human Cells

RENTA expression was assessed in human 293T cells transiently transfected with pTHr-RENTA or infected with MVA.RENTA using immunofluorescence and immunoblotting (Western blotting).

Immunofluorescence: For the immunofluorescence studies, six-well plates containing sterile slides pre-treated with poly-L-lysine (70,000-150,000 molecular mass; Sigma) were seeded with 293T cells (2×10⁵ cells per slide). Twenty four hours later, the cell monolayers were transfected with pTHr-RENTA or infected with MVA-RENTA at an MOI of 5. After a 24-hour incubation at 37° C. with 5% CO₂, the cells were washed and their membranes were perforated. The slides were blocked with 2% FCS in phosphate-buffered saline (PBS) at 4° C. for 1 hour and incubated with a 1:200 dilution of the designated primary mAb at 4° C. overnight. The mAbs were against the Pk tag (Serotec, Oxford, UK), Nef, RT or Tat (EVA352, EVA3019 and EVA3106, respectively, provided by Centralized Facility for AIDS Reagents UK). After incubation, the slides were washed once in PBS and incubated at 4° C. overnight with a 1:500 dilution of an Alexa Fluor® 594-conjugated anti-mouse secondary antibody (Molecular Probes, Oregon, USA). The slides were again washed once with PBS, stained with DAPI (4,6-diamidino-2-phenylindole 2HCl) nuclear stain (in Vectashield® mounting medium, Vector Laboratories, USA) and photographed on a Zeiss immunofluorescence microscope at 40× magnification. For the localization studies, the slides were incubated with FITC-conjugated anti-GM130 or anti-CD63 antibodies at 4° C. overnight after incubation with the anti-Pk mAb. Following this third incubation, the slides were washed with PBS and examined on a confocal microscope.

The immunofluorescence results demonstrate that RENTA expression is detectable in human 293T cells transfected with pTHr.RENTA using mAbs against HIV Tat, RT, Nef and Pk as well as in human 293T cells infected with an MVA.RENTA using a mAb against Pk (FIG. 15). For localization studies, human 293T cells were transfected with pThr.RENTA and stained with the anti-Pk mAb followed by anti-CD63 antibody, a lysosomalalate endosomal marker or stained with the anti-Pk mAb followed by anti-GM 130 antibody, a Golgi matrix marker. The results indicate that RENTA (as assessed by the location of the Pk epitope) did not significantly co-localize with the lysosomal marker but rather appears to accumulate largely in the Golgi apparatus.

Immunoblotting: To detect RENTA expression by immunoblotting, human 293T cells were either transiently transfected with pTHr.RENTA or infected with MVA.RENTA and lysed 48 hours later in the presence of protease inhibitors. Individual polypeptides of the cell lysates were separated on SDS-polyacrylamide gels crosslinked with 15% N,N-diallyltartardiamide (DATD) using thin (0.75 mm) mini-slab gels from the Bio-Rad electrophoresis system. The separated polypeptides were transferred onto a nylon filter (Amersham International) using a semidry gel electroblotter (LKB), blocked with 209, Marvel (non-fat powdered milk) in PBS and incubated with anti-Pk mAb in PBS with 5% Marvel. Bound antibodies were detected using horseradish peroxidase (HRP)-conjugated protein A (Amersham International) in PBS with 5%, Marvel followed by enhanced chemiluminiscence detection (ECL; Amersham International).

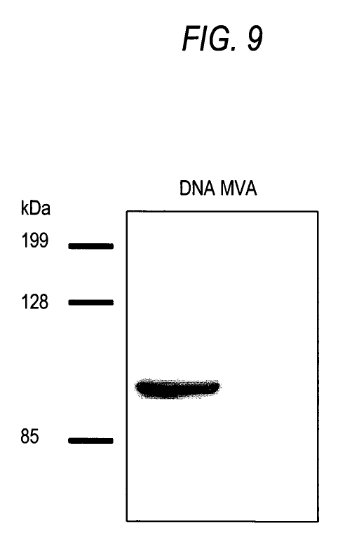

For cells transfected with pTHr.RENTA, the anti-Pk mAb detected a full-size protein of a predicted relative molecular mass of 99.4 kDa, suggesting that the majority of RENTA is not degraded (FIG. 9, left lane). However, RENTA expression in MVA-infected cells was not detected by immunoblotting (FIG. 9, right lane), a result previously experienced with some proteins expressed from recombinant MVAs (Hanke et al, (1998c) J. Gen. Virol. 79:83-90; Hanke 2000a).

### Example 3

### RENTA Characterization

Genetic stability of MVA.RENTA: The genetic stability of the inserted RENTA ORF and β-gal genes was confirmed by seven blind sequential passages of the MVA.RENTA in CEF cells. The original (passage 0) and the final (passage 7) virus stocks were then used to infect duplicate wells, of which one well was stained with neutral red and the other with 5-bromo-4-chloro-3-indolyl-β-D-galactopyranoside (X-gal) to detect MVA plaques (both empty MVA and MVA.RENTA) and the inserted (β-gal gene, respectively (Table 2). Comparison of the two titers suggested that MVA.RENTA was stable above the sensitivity of this assay. Immunofluorescence analysis of CEF cells infected with viral stocks from passages 0 and 7 indicated that the expression levels of RENTA were comparable.

TABLE 2
The Genetic Stability of MVA.RENTA

| | Blind Passage 0 | Blind Passage 7 |
|---|---|---|
| Experiment 1 | | |
| Neutral Red | 132^{a} | 60 |
| X-gal | 146 | 66 |
| Experiment 2 | | |
| Neutral Red | 175 | 104 |
| X-gal | 186 | 86 |
| | 165 | 90 |
| | 199 | 95 |
| Total Experiment 1 + 2 | | |
| Neutral Red | 493 | 250 |
| X-gal | 510 | 251 |

^{a}Data are expressed as numbers of plaques per well.

Inactivation of Tat NLS in RENTA: To determine whether the NLS deletion in the HIV tat domain affects the subcellular localization of RENTA, human 293T cells were transiently transfected with pTHr.RENTA and pTHr.RTNA as described in Example 2. The plasmid pTHr.RTNA encodes an HIV immunogen designated as RTNA that has domains from the HIV proteins Rev, Tat and Nef, with the sequences being from a consensus HIV Clade A sequence. The Tat protein in RTNA contains the NLS sequence. Subcellular localization of RENTA and RTNA was determined by immunofluorescence as described in Example 2 by staining with the anti-Pk mAb followed by anti-CD63 antibody or with the anti-Pk mAb followed by anti-GM 130 antibody. The results show that RENTA, with a mutated Tat, is not found in the nuclear compartment whereas RTNA, with a wild-type Tat, was readily found in the nuclei.

Lack of Tat TransactivationActivity in RENTA: To assess RENTA's transactivation activity, the ability of the HIV tat domain to activate expression of a CAT gene under control of the HIV-1 LTR was measured. Six-well plates seeded with human 293T cells (5×10⁵ cells per well). Twenty-four hours later, the cells were transfected with 5 μg of DNA per plasmid using SuperFect© transfection Reagent as recommended (Qiagen, Germany). A further 24 hours later, the cells were washed once with PBS, scraped from the wells, resuspended in 2 ml of 0.25 M Tris-HCl, pH 7.5 and subjected to 3 freeze-thaw cycles using a methanol/dry ice mixture and a 37° C. water bath. The lysates were chilled on ice and the supernatant collected by centrifugation for 5 minutes at 240 g at 4° C. CAT activity in 50 μl of cell lysates was assessed by the econofluor diffusion method (Morency et al., (1987) Biotechniques 5: 444-447, 1987).

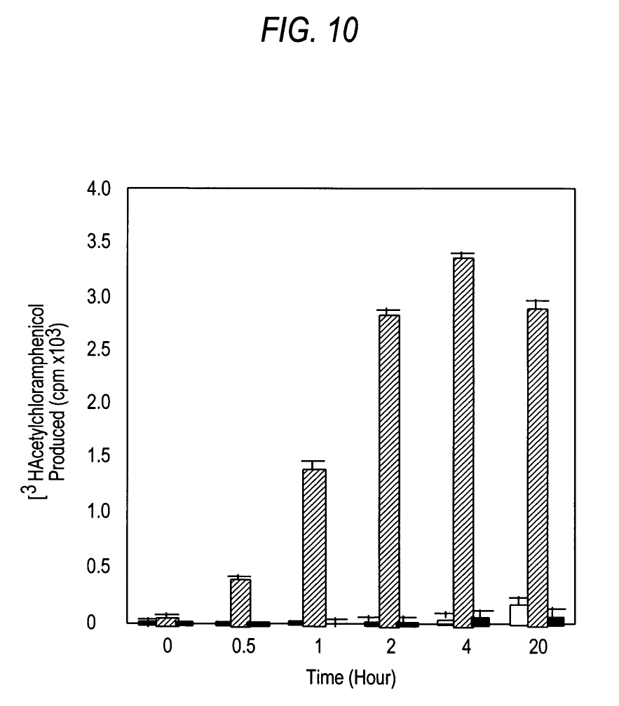

The cells were transfected with (1) plasmid pOGS210 containing wild-type HIV-1 LTR fused to CAT reporter gene, as a negative control (LTR-CAT only); (2) plasmids pOGS210 and pOGS213, containing a wild-type tat gene under control of a CMV promoter, as a positive control (LTR-CAT and CM V-Tat); or (3) plasmids pOGS210 and pThr.RENTA, with a mutated tat domain (LTR-CAT and CMV-RENTA) (Adams et al., (1988) Nucleic Acid Res. 16: 4287-4298). The results in FIG. 10 demonstrate that LTR-CAT only (as a negative control) produces little or no activity (white box), that wild type Tat transactivates CAT expression from the LTR-CAT plasmid (grey boxes) and that RENTA does not transactivate CAT expression from the LTR-CAT plasmid (black box), where the levels of CAT activity are comparable to the negative control.

Lack of CD4 and HLA class I down regulation by RENTA: HIV Nef downregulates the cell surface expression of CD4 and HLA class I molecules. To demonstrate that the HIV nef domain of RENTA lacks this activity, the RENTA ORF was subcloned as described in Example 1 into pIRES2.EGFP to produce the bi-cistronic plasmid pIRES2.RENTA-EGFP expressing green fluorescent protein (GFP) and RENTA. RENTA expression from this plasmid was confirmed by immunofluorescence. To assess cell surface expression of CD4 and the HLA class 1 molecules, 5×10⁶ human PBMCs were transfected with six μg of (1) pIRES2-EGFP, (2) pIRES2.Nef.EGFP or (3) pIRES2.RENTA.EGFP using the Nucleofector™ technology as recommended (Amaxa Biosystems, Germany). Forty-eight hours post-transfection, cells were co-stained with phycoerythrin-conjugated anti-CD4 mAb (Pharmingen) and allophycocyanin-conjugated anti-HLA-A, B, C mAb (Pharmingen), fixed and stored at 4° C. until use. The labeled cells were analysed by flow cytometry (FACS) using the CellQuest software (BD Biosciences, UK).

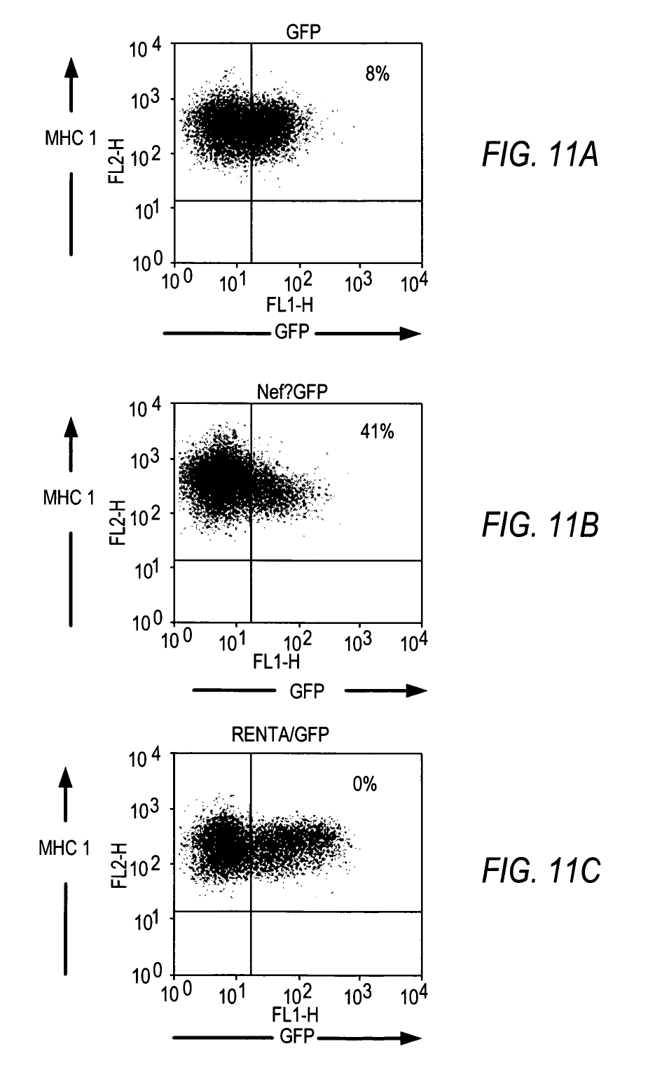

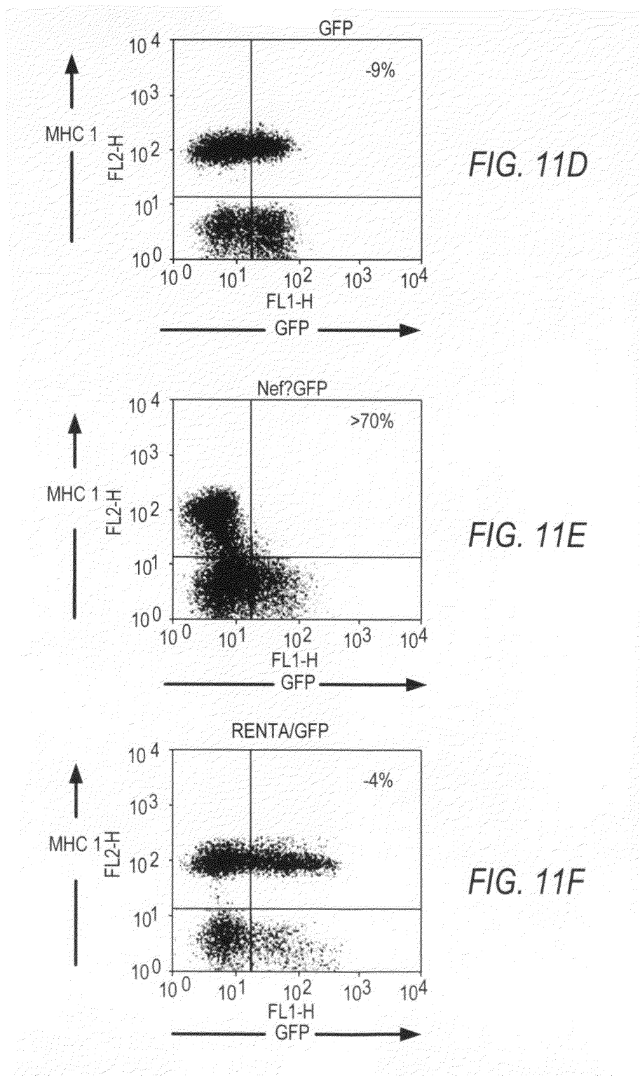

The PBMCs expressing GFP alone (FIG. 11, left panels) or GFP/RENTA (FIG. 11, right panels) did not down regulate the surface expression of HLA class I and CD4 molecules, whereas the PBMCs expressing GFP-Nef did (FIG. 11, middle panels). In FIG. 11, HLA Class I expression is shown in the upper panels and CD4 expression is shown in the lower panels. Thus, RENTA contains an HIV nef domain incapable of down regulating cell surface expression of CD4 and HLA class I molecules.

### Example 4

### RENTA Immunogenicity in Mice

Plasmid or MVA stimulated-immunity: The immunogenicity of the pTHr-RENTA and MVA-RENTA was assessed in mice using the pb9 epitope. Two groups of 5-6 week-old female BALB/c mice were injected into the anterior tibial muscles with 50 μg of endotoxin-free pTHr.RENTA in PBS or with 10⁶ pfu MVA.RENTA under general anesthesia. Ten days later, the animals were sacrificed and their spleens were removed. Individual spleens were processed through a cell strainer (Falcon) using a 2-ml syringe rubber plunger. The splenocytes from each animal were washed twice and suspended in 10 ml of lymphocyte medium (RPMI 1640 supplemented with 10% FCS penicillin/streptomycin, 20 mM HEPES and 15 mM 2-mercaptoethanol). Two ml of splenocyte suspension was used for the IFN-γ ELISPOT assay and the rest for a bulk CTL culture. All animal procedures and care strictly conformed to the U.K. Home Office Guidelines.

To prepare the bulk CTL cultures, 8 ml of the splenocyte suspension were incubated with 2 pg/ml of pb9 peptide in an humidified incubator in 5% C0₂ at 37° C. for 5 days. On the day of the CTL assay, the cells were washed 3× with RPMI and resuspended at 10⁷ cells per ml in R10 (RPMI 1640 supplemented with 10% FCS and penicillin/streptomycin) for use as effector cells in a ⁵¹Cr-release assay.

For each batch of splenocytes, the effector cells were diluted 2-fold in U-bottom wells of a 96-well plate (Costar) using R10 medium to yield effector to target ratios between 200:1 to 3:1 after addition of the target cells. Five thousand ⁵¹Cr-labeled P815 target cells in R10 medium with or without 2 pg/ml of pb9 peptide were added to the effectors and the mixture was incubated at 37° C. for 5 hours. Spontaneous and total chromium releases were estimated from wells containing target cells in medium alone or in medium with 5% Triton X-100, respectively. The percentage specific lysis was calculated as [(sample release-spontaneous release)/(total release-spontaneous release)]×100. The spontaneous release was lower than 5% of the total counts per minute.

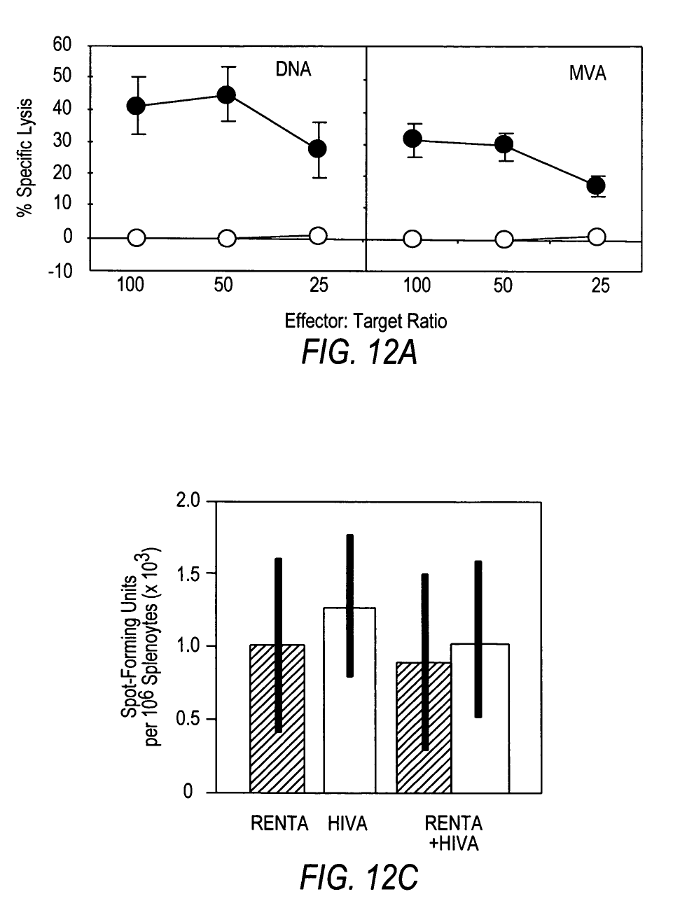

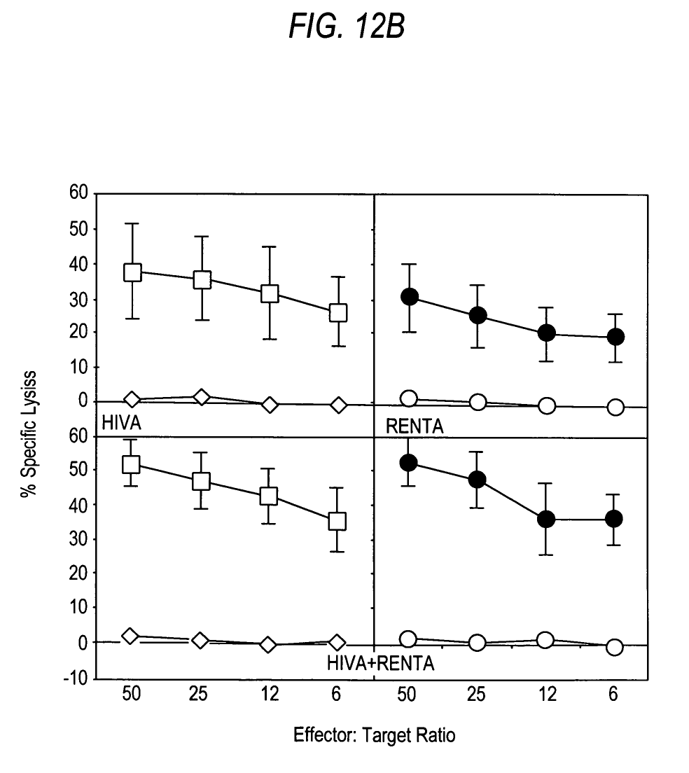

In FIG. 12A, the left panel shows the results for mice immunized with pTHr.RENTA and the right panel shows the results for mice immunized MVA.RENTA in the ⁵¹Cr-release assay with peptide-pulsed (solid circle) or unpulsed (open circle) target cells. All animals responded to the immunization and relatively high levels of lytic activities were detected.

### Example 5

### DNA Prime-MVA Boost Regimens in Mice

HIVA or RENTA alone: BALB/c mice were immunized and splenocytes isolated as described in Example 4 using 25 μg of endotoxin-free pTHr.HIVA in PBS on day 0, followed by 10⁶ pfu of MVA.HIVA on day 14, and sacrifice of the animals on day 24. Splenocytes for bulk CTL culture were prepared as in Example 4, but incubated in the presence of a HIVA specific CTL peptide, the P18-I10 epitope peptide having the amino acid sequence RGPGRAFVTI (Takahashi et al., (1988) Proc. Natl. Acad. Sci. USA 85:3105:3109). The ⁵¹Cr-release assays were conducted as in Example 4 using the P18-I10 peptide for the peptide pulse. The same protocol was followed for RENTA alone, using a pTHr.RENTA prime, MVA.RENTA boost and the pb9 peptide for bulk CTL culture and the peptide pulse.

Mixed HIVA/RENTA: BALB/c mice were immunized and splenocytes isolated as described in Example 4 using 25 μg each of endotoxin-free pTHr.HIVA and pTHr.RENTA in PBS on day 0, followed by 10⁶ pfu each of MVA.HIVA and MVA.RENTA on day 14 and sacrifice of the animals on day 24. Splenocytes for bulk CTL culture were prepared as in Example 4, but incubated in the presence of the HIVA P18-I10 peptide and/or the MVA pb9 peptide. In vitro restimulation can be done together as each peptide is presented by a different MHC. The ⁵¹Cr-release assays were conducted as described in Example 4 using the P18-I10 peptide for the peptide pulse for HIVA detection or the pb9 peptide for the peptide pulse for MVA detection.

ELISPOT Assays: The IFN-γ ELISPOT assay was carried out using the Mouse IFN-γ Secreting Cell Kit (BD Biosciences, UK) according to the manufacturer's instructions. In brief, 10⁵ isolated splenocytes depleted of red blood cells were restimulated in duplicate in anti-IFN-γ-precoated 96-well plates with R10 medium alone, R10 supplemented with concanavalin A at 4 μg/ml or R10 with the indicated peptide at 2 μg/ml for 18 hours at 37° C. in 5% CO₂. Following lysis of the cells by a 10-minute incubation in ice water, spots were visualized using sequential applications of a biotin-conjugated secondary anti-IFN-γ antibody, avidin-horseradish peroxidase and AEC (3-amino-9-ethyl-carbazole, Sigma, UK) and H₂O₂ (30%). Spots were counted using an ELISPOT reader (Autoimmun Diagnostika GmbH, Germany) and expressed as spot-forming units per 106 splenocytes.

Results: The elicited immune responses from the various prime boost regimens are shown in FIG. 12B for ⁵¹Cr-release assays and in FIG. 12C for the ELISPOT assays. T-cell responses against the HIVA P18-I10 epitope are shown by diamonds and against the RENTA pb9 epitope by circles. In FIG. 12B, (1) the upper left panel shows the HIVA only prime-boost pulsed with the P18-I10 peptide (closed) or unpulsed (open); (2) the upper right panel shows the RENTA only prime-boost pulsed with the pb9 peptide (closed) or unpulsed (open); (3) the lower left panel shows the mixed HIVA/RENTA prime-boost pulsed with the P18-I10 peptide (closed) or unpulsed (open); and (4) the lower right panel shows the mixed HIVA/RENTA prime-boost pulsed with the pb9 peptide (closed) or unpulsed (open). FIG. 12C shows the IFN-γ production stimulated by the pb9 peptide for RENTA (hatched box) or by the P18-I10 peptide for HIVA (open box) for each of the three prime boost regimens, from left to right, RENTA only, HIVA only or mixed HIVA/RENTA.

In these experiments, the respective HIVA and RENTA epitopes were approximately equally immunogenic and in the bulk peptide-restimulated cultures induced similar lytic activities (FIG. 12B) and comparable numbers of spot-forming units producing IFN-γ upon peptide stimuli (FIG. 12C). No decrease in these effector functions were observed upon combination (FIGS. 12B and 12C).

### Example 6

### Demonstration of Broad Murine T-Cell Responses

The breadth of T-cell responses induced against the RENTA immunogen when used together with the HIVA vaccines in the BALB/c mouse was examined using mice immunized via the MM HIVA/RENTA prime-boost protocol of Example 7. Induction of specific immune responses to three known RENTA epitopes was demonstrated using an ex vivo intracellular cytokine staining assay. For this assay, isolated mouse splenocytes were stimulated with the appropriate HIVA or RENTA peptide- or RENTA peptide pool-pulsed P815 cells in the presence of anti-CD28/anti-CD49d mAbs for 90 minutes at 37° C. in 5% CO₂. Brefeldin A was then added to inhibit cytokine secretion and the samples were incubated for additional 6 hours before terminating the reaction with EDTA and FACS fix solution. The cells were permeabilized and incubated with PE-conjugated anti-CD8 and FITC-conjugated anti-IFN-γ mAbs (BD PharMingen) and analyzed using FACS.

The results in Table 3 demonstrate the multi-specificity of CTL induced by RENTA where the percentage of CD8+ splenocytes producing IFN-γ are shown for naive (unimmunized) mouse splenocytes and for mixed HIVA/RENTA (MM) mouse splenocytes stimulated with P18-110 peptide, pb9 peptide, RT1 peptide, RT2 peptide and the three peptide pools RENTA1, RENTA2 and RENTA3. The RT1 peptide has the sequence RAHLLSWGF and is from the N-terminal HIV reverse transcriptase domain of RENTA; the RT2 peptide has the sequence VYYDPSKDLI and is from the C-terminal HIV reverse transcriptase domain of RENTA. The peptide pools consist of 14-16-mer peptides overlapping by 11 amino acids across the entire RENTA immunogen, where the RENTA1 pool covers amino acids 2-100 and 262417 of RENTA, the RENTA2 pool covers amino acids 407-705 of RENTA and the RENTA3 pool covers amino acids 90-272 and 695-842 of RENTA.

TABLE 3
Ex Vivo Intracellular Cytokine Production

| Peptide/Pool | Naïve | MM |
|---|---|---|
| P18-I10 | 0.05^{a} | 10.1 |
| pb9 | 0.05 | 11.9 |
| RT1 | 0.08 | 9.05 |
| RT2 | 0.04 | 0.61 |
| RENTA1 | 0.10 | 0.82 |
| RENTA2 | 0.10 | 10.0 |
| RENTA3 | 0.10 | 0.65 |

^{a}Percentage CD8+ splenocytes producing IFN-γ.

### Example 7

### Effect of Physical Separation of Immunogens in a Prime-Boost Protocol

Immunizations: The effect of mixing the HIVA and RENTA immunogens in a prime-boost protocol was examined to assess the potencies of delivering the HIVA and RENTA immunogens into the same or separate hind legs. Groups of BALB/c mice were immunized i.m. on the same schedule as described in Example 5 by priming injections of 25 μg for each plasmid and boosting injections of 5×10⁴ pfu for each MVA as follows: pTHr.HIVA DNA and MVA.HIVA into the left leg and pTHr.RENTA DNA and MVA.RENTA into the right leg (SS), each plasmid into a separate leg and mixed MVAs into both legs (SM), mixed plasmids into both legs and each MVA into a separate leg (MS) or mixed plasmids and mixed MVAs into both legs (MM). Ten days after the second immunization, the mice were sacrificed, splenocytes isolated as generally described in Example 4 and the elicited immune responses were assessed using (1) an intracellular IFN-γ staining assay, (2) an H-2D^{d}/P18-I10 tetramers assay, (3) an IFN-ELISPOT assay, and (4) a ⁵¹Cr-release assay.

Assays: (1) The intracellular IFN-γ staining assay was conducted as described in Example 6 using the same peptides and peptides pools for ex vivo peptide restimulation. (2) The tetrameric MHC/peptide complexes for H-2D^{d}/P18-I10 tetramers were prepared using standard procedures (Hanke, 1999). Briefly, both heavy and light H-2D^{d} chains were expressed in H. coli strain BL-21, purified from inclusion bodies, denatured in 8 M urea and refolded in the presence of the P18-I10 peptide. The complex was biotinylated using the BirA enzyme (Avidity) and purified on fast-performance liquid chromatography (FPLC) and monoQ ion-exchange columns. The formation of tetrameric complexes was induced by addition of chromogen-conjugated streptavidin (ExtrAvidin®; Sigma) to the refolded biotinylated monomers at molar ratio of MHC-peptide monomer:PE-streptavidin of 4:1. Labeled tetrameric complexes were stored in the dark at 4° C. until use (as described in Hanke 1999). The assay was performed by incubating unrestimulated splenocytes (fresh or thawed) with 1 μg tetrameric complex for 20 min at 4° C., incubating a further 5 min on ice, adding 1 μg each anti-CD3 and anti-CDS mAbs (each conjugated to a different color agent) and incubating for another 20 min on ice. The cells were washed twice, fixed in formaldehyde and analyzed by FACS as described in Example 3.

(3) The IFN-γ ELISPOT assay was conducted as described in Example 5 using the RT1 and RT2 peptides described in Example 6.

(4) For ⁵¹Cr-release assay, bulk CTL cultures were prepared as described in Example 4 by incubating with one of the P18-I10, pb9, RT1 or RT2 peptides. The ⁵¹Cr-release assays were also conducted as described in Example 4 using the target cells pulsed with the peptide used to prepare the bulk CTL.

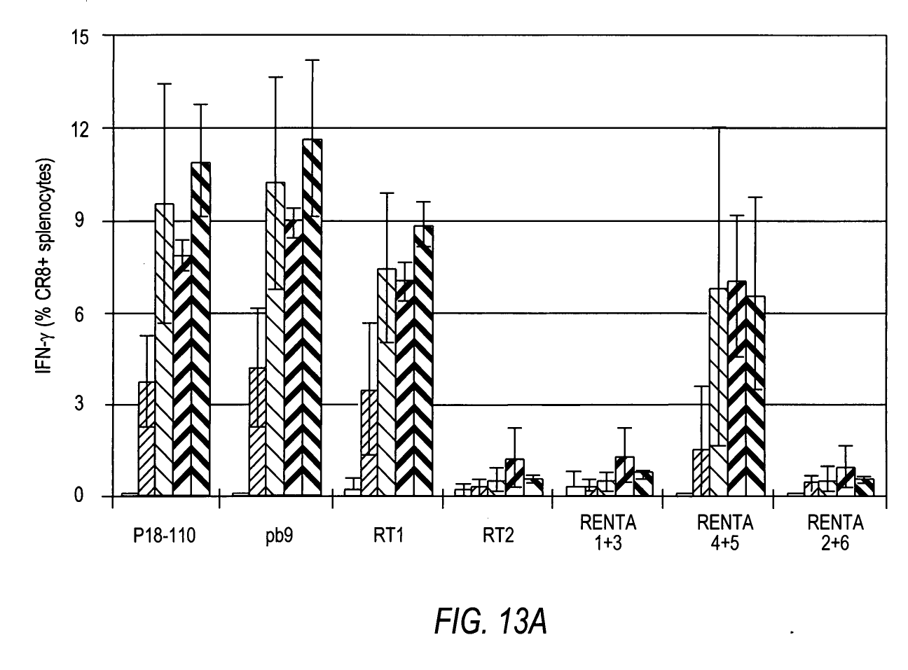

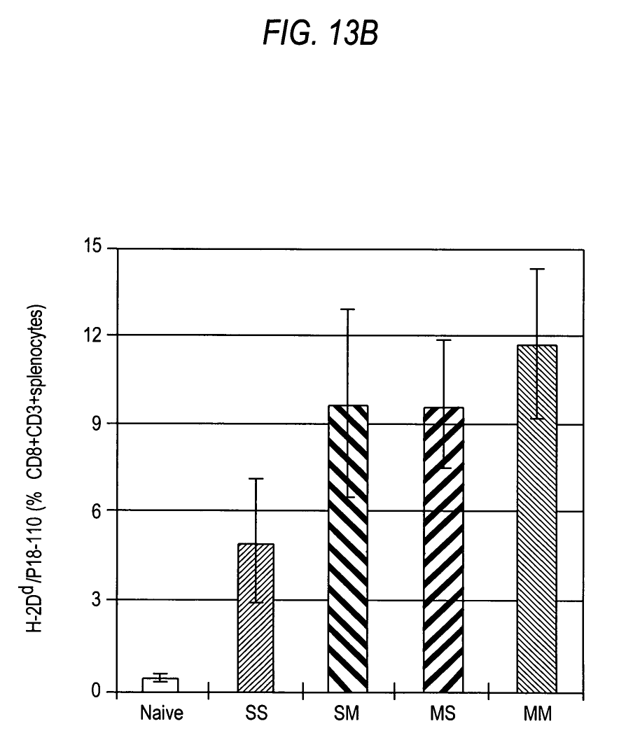

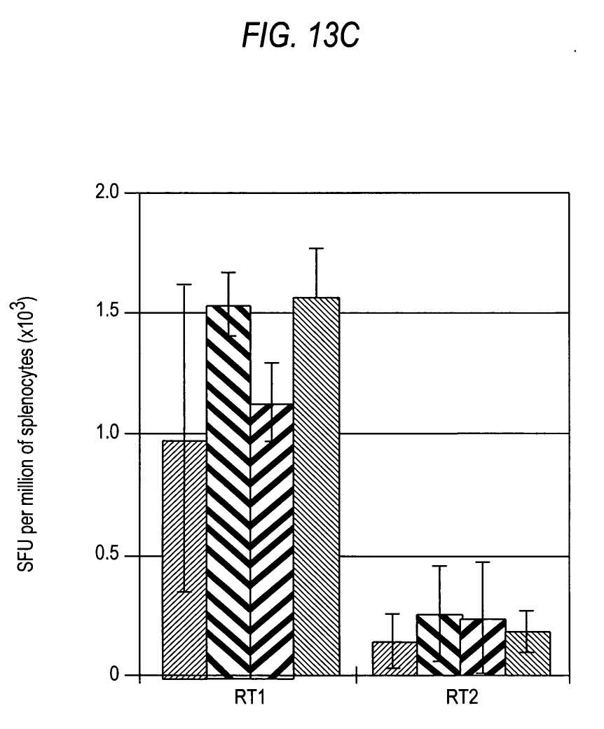

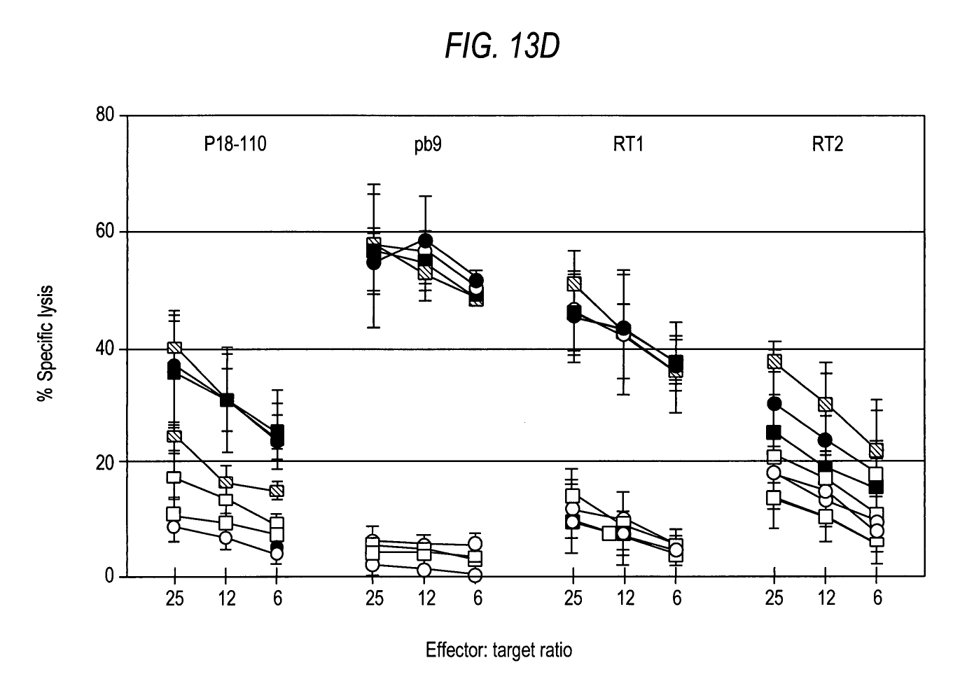

Results: The results for the four assays are shown in FIGS. 13A-13D. For each panel, the immunization regimen is depicted as naive, open box; SS, narrow upward diagonal box; SM, wide downward diagonal box; MS, wide upward diagonal box; and MM, narrow downward diagonal box. Panel A shows the percentage of CD8+ cells producing IFN-γ for the indicated peptides or peptide pools. Panel B shows the percentage of CD3+ and CD8+ cells reactive with H-2D^{d}/P18-I10 tetramers. Panel C shows relative IFN-y production as SPU in the ELISPOT assay for with the indicated peptides. Panel D shows the ⁵¹Cr-release assay using regimes SS (grey circles), SM (grey squares) MS (black circles) and MM (black squares) and target P815 cells unpulsed (open) or pulsed (solid) with peptides indicated at the top of the graphs. For all assays, splenocytes from individual mice were treated separately and the results are expressed as an arithmetic mean+standard deviation of a particular treatment group.

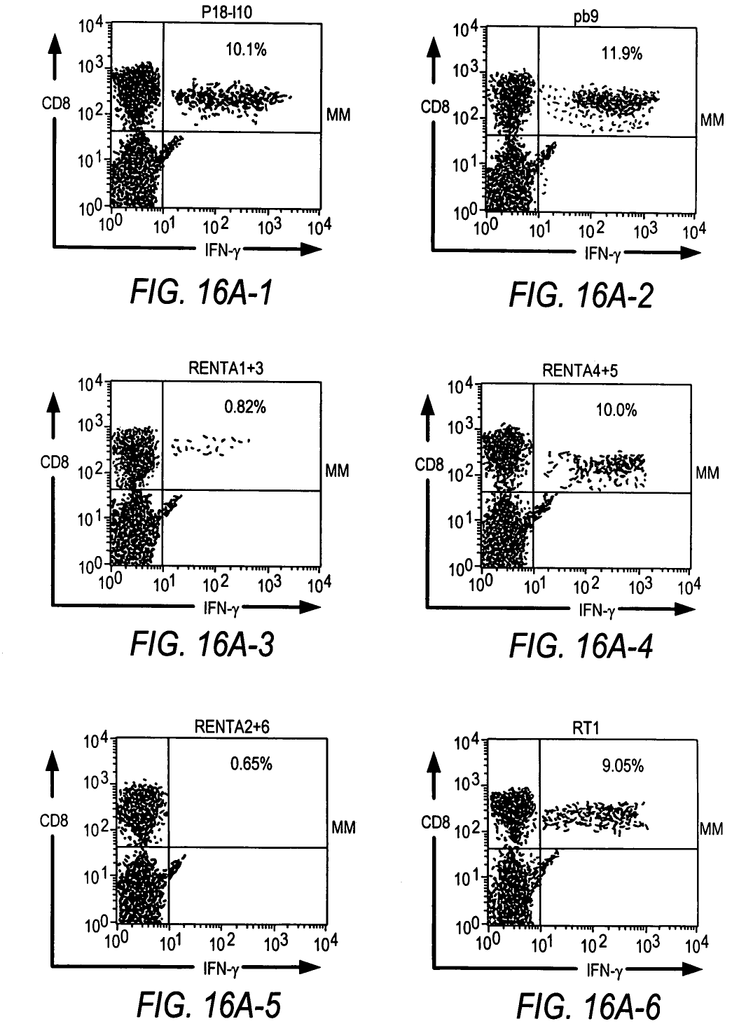

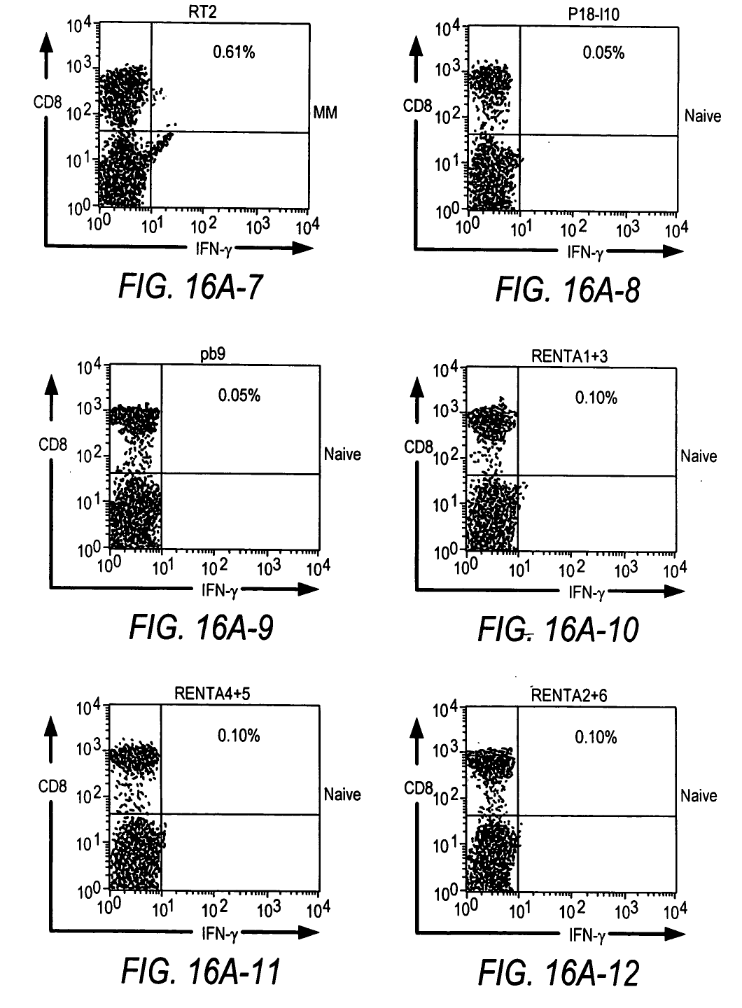

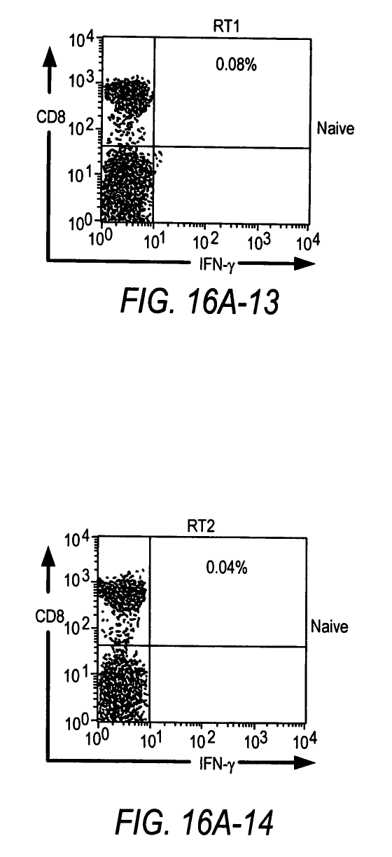

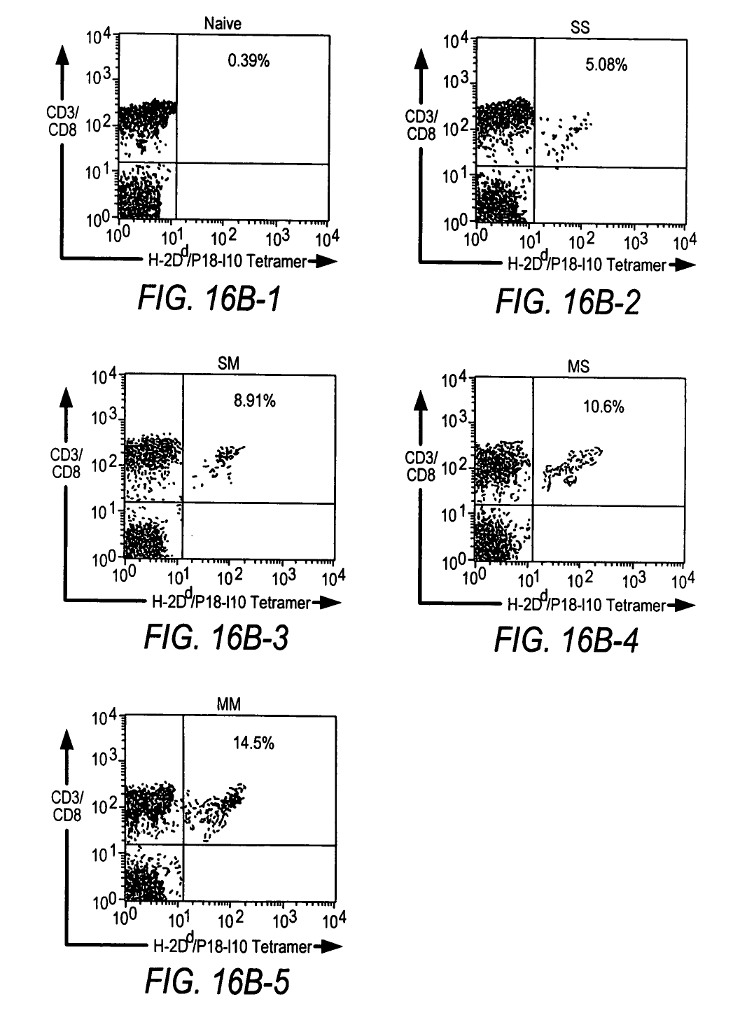

The observed frequencies of IFN-γ-producing cells upon peptide restimulation in vitro suggests that immunogen mixing provides an advantage over separate delivery with the immunogenicities ranking as SS<SM=MS<MM (FIG. 13A). This hierarchy was also seen analyzing the H-2D^{d}/P18-I10 tetramer reactivities (FIG. 13B), a similar trend was suggested by the IFN-γ ELISPOT assay (FIG. 13C), but could not be seen in the ⁵¹Cr-release assay, which, however, expands the memory cells for 5 days in vitro and might thus obscure initial cell number differences (FIG. 13D). Examples of the intracellular cytokine and tetramer staining of representative mice are shown in FIG. 16, panels (a) and (b), respectively. Thus, mice immunized using the combined DNA-MVAIHIVA-RENTA responses to at least five distinct T-cell epitopes: P18-I10 of HIVA, and pb9 and three peptide pools of RENTA.

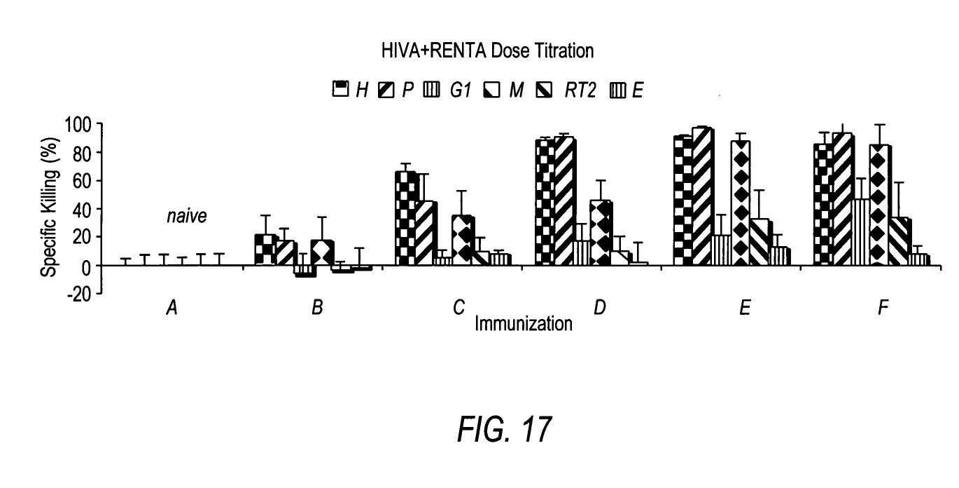

Immunogenicity of a single delivery of either the pTHr.RENTA and MVA.RENTA vaccines and their prime-boost combinations were tested in the BALB/c mice. T-cell responses were assessed in an in vivo killing assay using transferred, differentially labeled peptide-pulsed targets, which were reisolated after 12 hours and enumerated in a FACS analysis. FIG. 17 shows that immune responses induced by HIVA were broadened by co-administration of RENTA.

### Example 8

### Immunogenicity in Non-Human Primates

Rhesus macaques (Macaca mulatta) positive for the Mamu-A*01 allele of MHC class I were immunized with a DNA prime-MVA boost regimen. Three macaques (monkeys 1-3) received immunizations with plasmids pTHr.HIVA and pTHr.RENTA at weeks 0 and 4 followed by immunization with recombinant MVA.HIVA and MVA.RENTA at weeks 20 and 24. Two macaques (monkeys 4 and 5) received the same priming immunizations but were boosted with recombinant MVA.HIVA and MVA.RENTA at weeks 8 and 12. The immunizations consisted of 1 mg of each plasmid in 0.5 ml of 140 mM NaCl, 0.5 mM Tris-HCl, pH 7.7 and 0.05 mM EDTA delivered i.m. or 5×10⁷ pfu of each MVA in 0.1 ml of 140 mM NaCl and 10 mM Tris-HCl, pH 7.7 delivered intradermal (i.d.). The HIVA vaccines were delivered into the animals' arms and the RENTA vaccines into thighs. All immunizations and venipunctures were carried out under sedation with ketamine and the animals were regularly clinically examined. All procedures and care strictly conformed to the U.K. Home Office Guidelines.

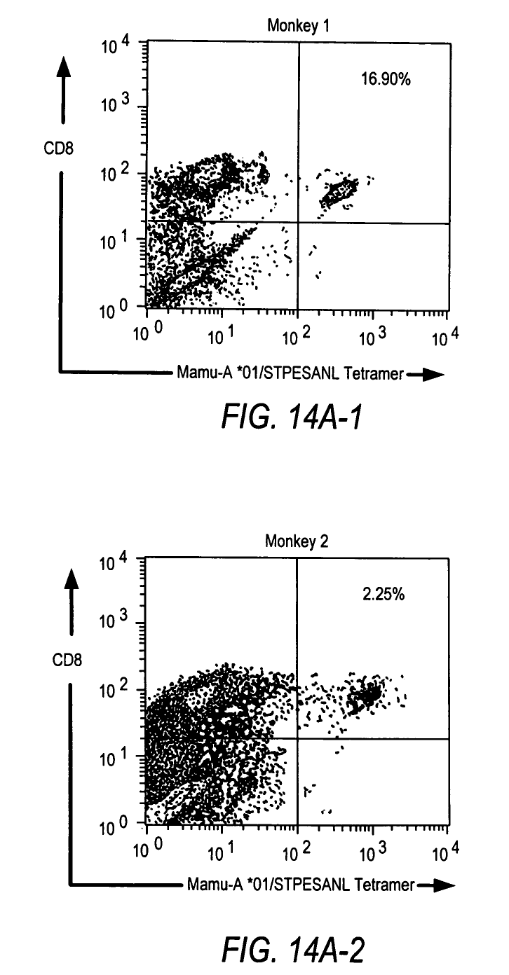

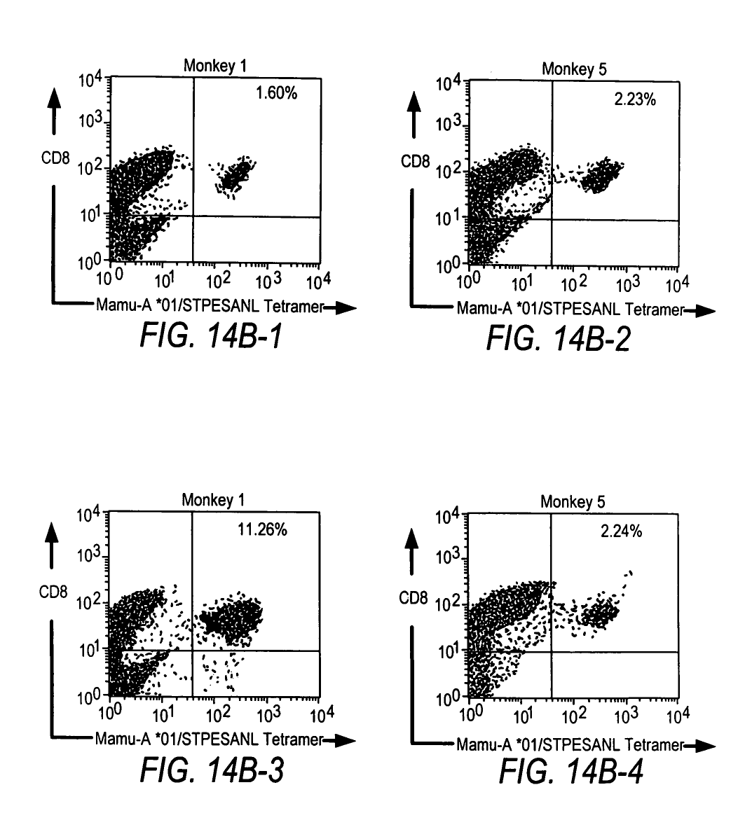

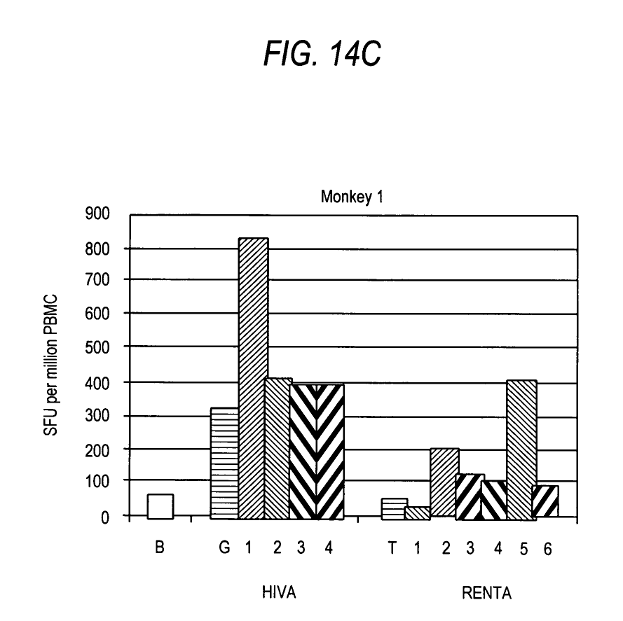

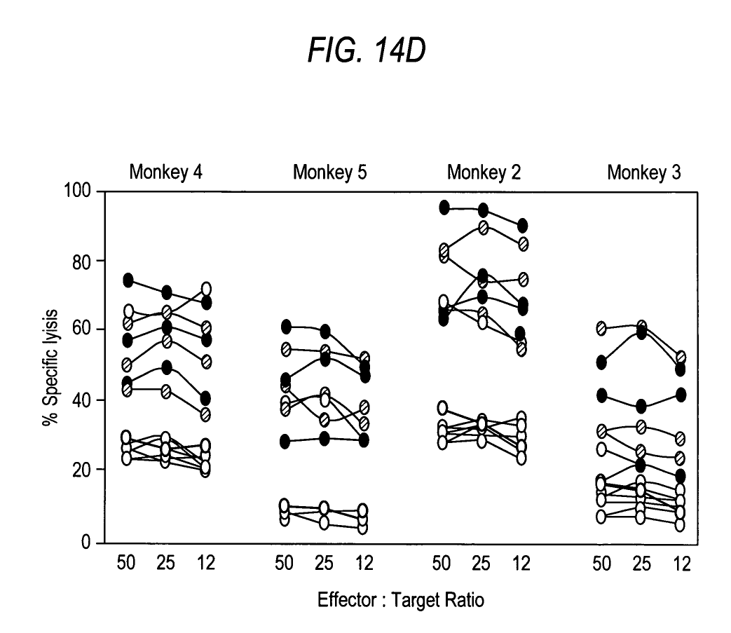

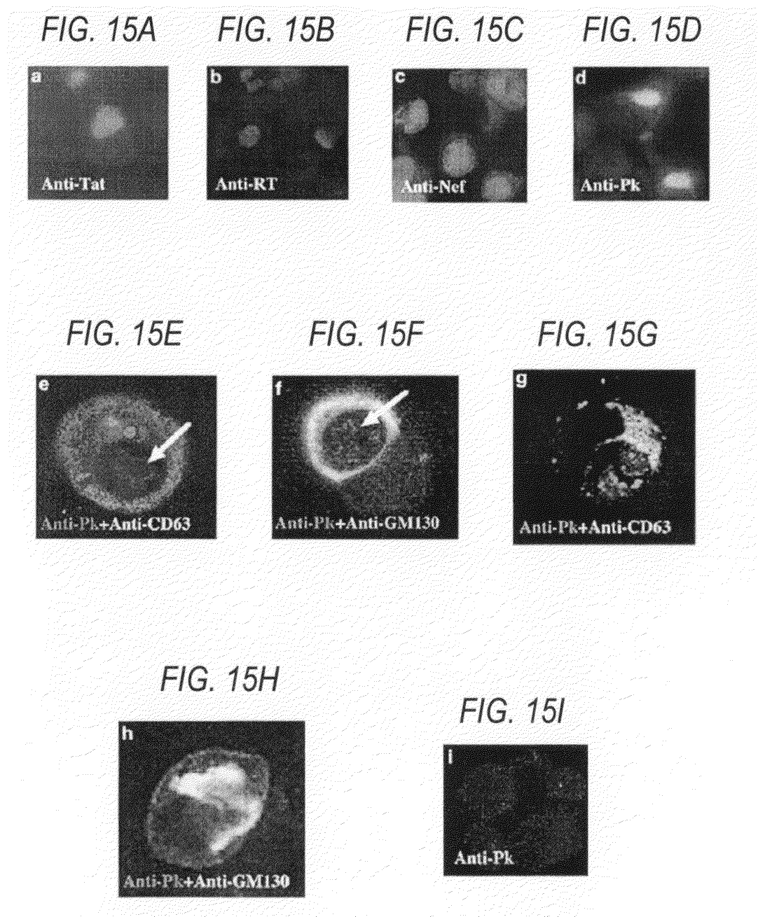

Monkey PBMC were isolated from heparinized blood using the Lymphoprep™ cushion centrifugation (Nycomed Pharma AS). PBMCs were cultured for 2 weeks with peptides derived from the SIV Gag (CTPDYNQM; HIVA) or Tat (STPESANL; RENTA) proteins for peptide-specific expansion. Tetrameric MHC/peptide complexes for Mamu-A*01/Gag or Mamu-A*01/Tat were prepared as described in Example 7. Immunogenicity was assessed using PBMCs restimulated with the Gag or Tat peptide for 2 weeks at 37° C., 5% CO₂ with an addition of huIL-2 on day 3. On the day of the assay, the cells were reacted with PE-conjugated Mamu-A*01/peptides tetrameric complexes and mouse anti-huCD8-PerCP mAb (BD PharMingen) and analyzed by FACS as described in Example 3. Examples of MHC/peptide tetramer reactivities after DNA prime alone (in blood drawn at week 16) are shown in FIG. 14A for monkeys 1 and 2. The tetramer reactivities after MVA boost are shown in FIG. 14B for monkeys 1 and 5 for blood drawn at weeks 22.

Using both the Mamu-A*01-restricted and overlapping peptides derived from the HIVA and RENTA immunogens, multi-specific responses were detected to both vaccines in an IFN-γ ELISPOT assay ex vivo (FIG. 14C). The IFN-γ ELISPOT assay was carried out on DNA primed-MVA boosted animals using freshly isolated PBMC (drawn at week 22) for both the Mamu-A*O1-restricted epitope peptides (G for Gag and T for Tat) and overlapping pools of peptides across the HIVA and RENTA proteins (numbers indicated below). The procedures and reagents of the MABTECH kit (Cat. No. 3420M-2A) were used. Briefly, PBMC were isolated on a Lymphoprep cushion and incubated at 37° C., 5% CO₂ for 24 hours with the indicated peptide or peptide pool. The released IFN-γ was captured by a mAb immobilized on the bottom of assay wells, visualized by combination of a second mAb coupled to an enzyme and a chromogenic substrate as described in Example 4. Spots were counted using an ELISPOT reader (Autoimmun Diagnostika GmbH, Germany) and expressed as spot-forming units per 10⁶ splenocytes. Only one animal is shown because of consistent high background/no-peptide signals. The HIVA Gag epitope, which is immunodominant during infection of Mamu-A*01+ animals with SIV, is not immunodominant in this setting (Wee et al., (2002) J. Gen. Virol. 83: 75-80).

For monkey bulk CTL cultures, 8×10⁶ isolated PBMC were restimulated with 10 μm peptide (or peptide pool) in 100 μl of R20 in 5% CO₂ at 37° C. for 1 hour and resuspended in total of 4 ml of R20 supplemented with 25 ng/ml of huIL-7 in two 24-well-plate wells. On day 3, Lymphocult-T (Biotest AG) was added to the final concentration of 10% (v/v). On day 8, 5×10⁶ peptide-pulsed irradiated autologous B lymphoblastoid cell lines (B-LCL) was added to the cultures followed by Lymphocult-T on day 11. Cytolytic tests were carried out on day 14.

For the ⁵¹Cr-release assay, the effector cells were diluted sequentially 2-fold in U-bottom wells 96-well plates (Costar) at effector to target ratios of 50:1, 25:1 and 12:1. Five thousand ⁵¹Cr-labelled autologous B-LCL pulsed (2 μg/ml) or unpulsed with peptide (tat or gag) or peptide pools (for HIVA or RENTA) were added to the effectors and incubated at 37° C. for 6 hours. Percent specific lysis was calculated as for the mouse lysis assays. Spontaneous release was for all samples below 20% of the total counts. The majority of animals responded to at least 7 different CTL epitopes: the Tat and Gag epitopes, HIVA epitopes from two peptide pools and RENTA epitopes from three peptide pools (FIG. 14D).

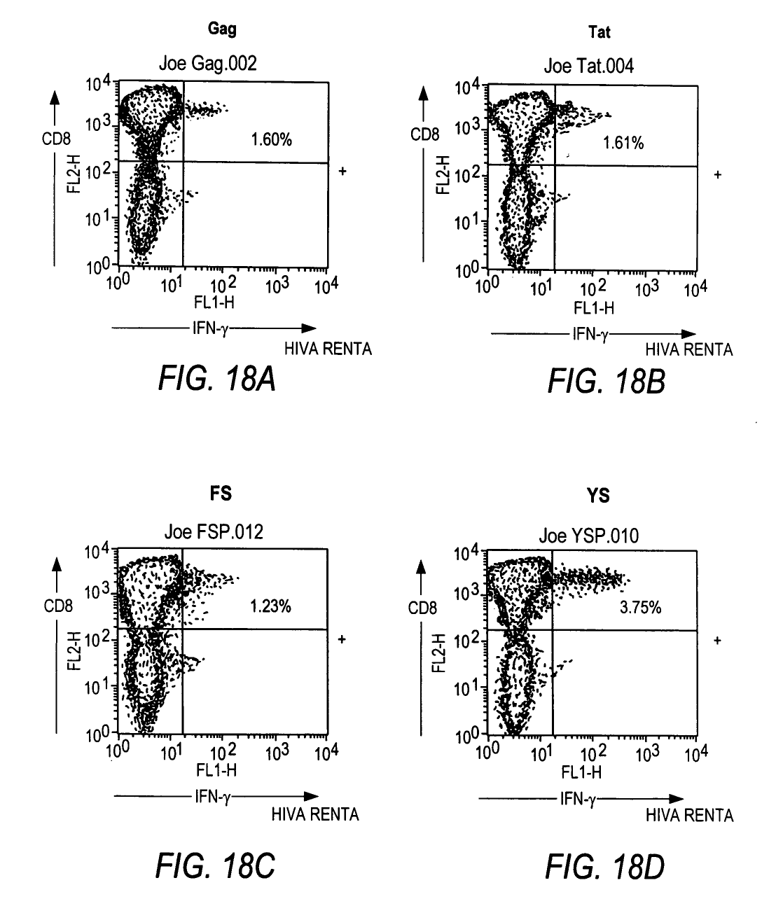

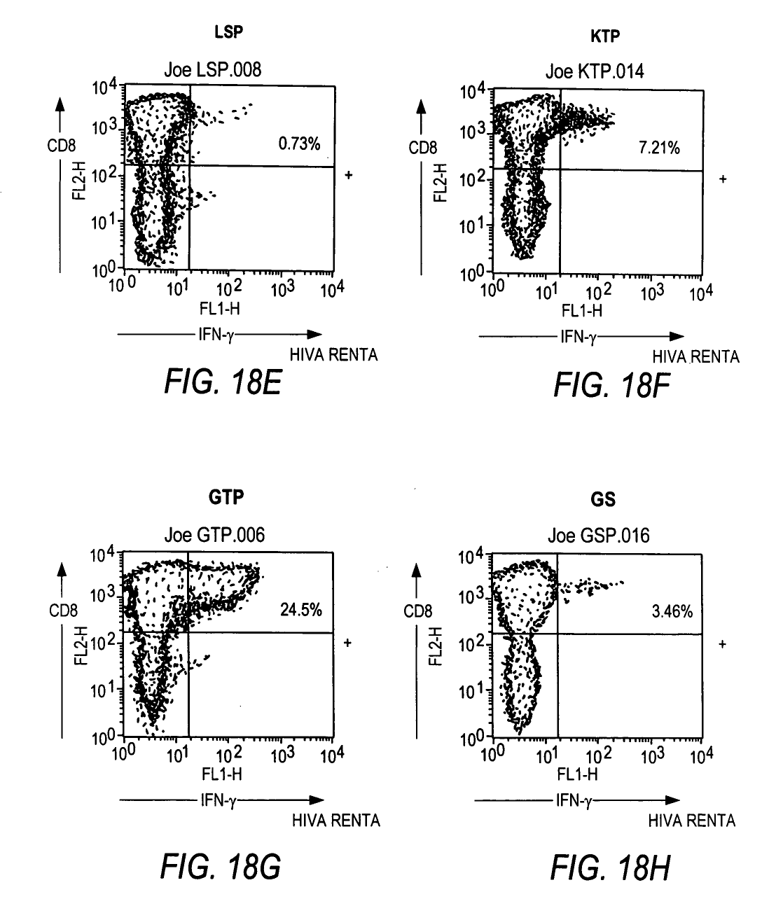

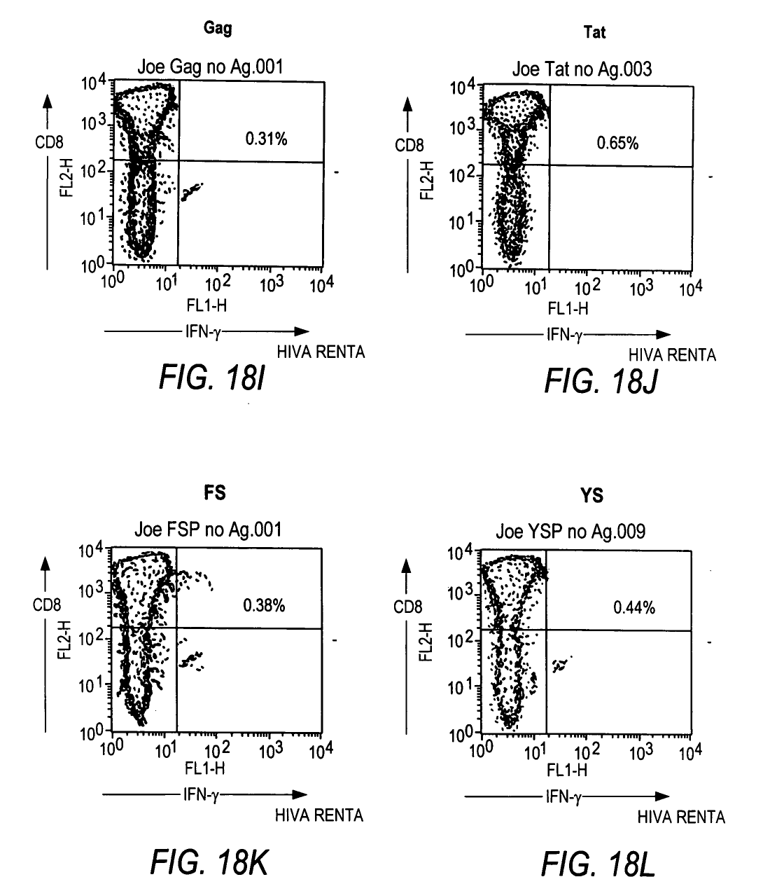

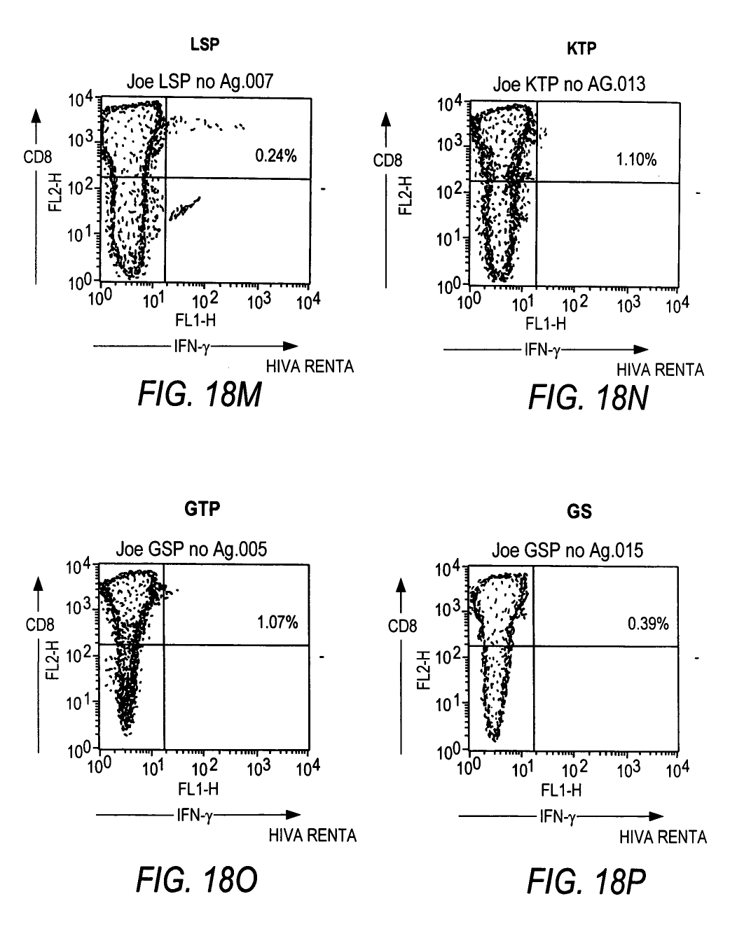

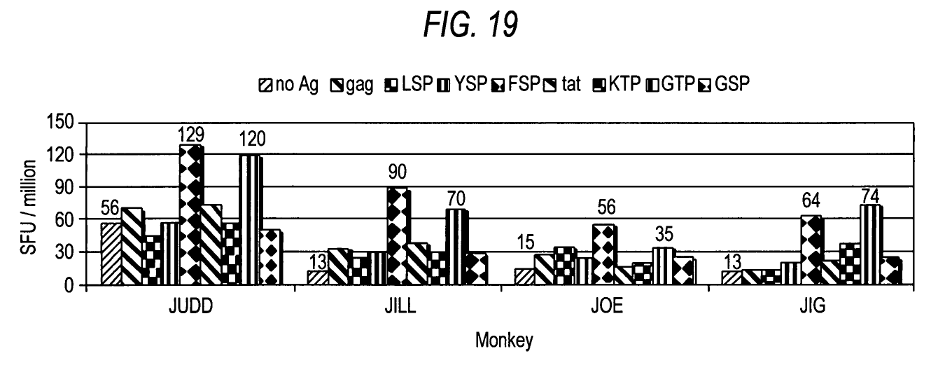

Further to the observation that RENTA and HIVA vaccines can be delivered together and induce multi-specific immune responses in rhesus macaque, frozen lymphocyte samples of week 36 were used and responses were detected in a total of 8 Mamu-A*01-restricted epitopes in one animal, 4 epitopes from HIVA, and 4 epitopes from RENTA (FIG. 18). Multi-specific HIVA- and RENTA-vaccine induced responses were still detectable one year after vaccine administration (FIG. 19).

### REFERENCES

1. Abath and Simpson (1990) Pept Res. 3(4): 167-8.
2. Abbas and Lichtman, Cellular and Molecular Immunology (2000) 4th Edition, W.B. Saunders Company, Philadelphia, Pa., p. 454.
3. Adams et al. (1988) Nucleic Acids Res. 16(10): 4287-98.
4. Adler et al. (1998) Med. Microbiol. Immunol (Berl). 187(2): 91-6.
5. Ahmad-Nejad et al. (2002) Eur. J. Immunol. 32(7): 1958-68.
6. Allcock (1998) Appl. Organometallic Chem. 12(10-11): 659-666.
7. Allen et al. (2000a) J. Immunol. 164(9): 4968-78.
8. Allen et al. (2000b) Nature 407(6802): 386-90.
9. Allen et al. (2002) J. Virol. 76(20): 10507-11.
10. Altenburger et al. (1989) Arch. Virol. 105(1-2): 15-27.
11. Amara et al. (2001) Science 292(5514): 69-74.
12. Andre et al. (1998) J. Virol. 72(2): 1497-503.
13. Barillari and Ensoli (2002) Clin Microbiol Rev. 15(2): 310-26.
14. Barouch et al. (2000) Science 290(5491): 486-92.
15. Berglund et al. (1998) Nat. Biotechnol. 16(6): 562-5.
16. Berman et al. (1990) Nature 345(6276): 622-5.
17. Boyer et al. (2002) J. Liposome Res. 12(1-2): 137-42.
18. Brake et al. (1984) Proc Natl Acad Sci USA. 81(15): 4642-6.
19. Broach et al. (1979) Gene 8(1): 121-33.
20. Chakrabarti et al. (1985) Mol. Cell. Biol. 5(12): 3403-9.
21. Chuang et al. (2002) J. Leukoc. Biol. 71(3): 538-44.
22. Clements-Mann et al. (1998) J. Infect Dis. 177(5): 1230-46.
23. Davis et al. (1993) Hum. Mol. Genet. 2(11): 1847-51.
24. Davis et al. (1994) Vaccine 12(16): 1503-9.
25. Dorrell et al. (2000) AIDS14(9): 1117-22.
26. Egan et al. (1995) J Infect Dis. 171(6): 1623-7.
27. Fultz et al. (1992) Science 256(5064): 1687-90.
28. Gaschen et al. (2002) Science 296(5577): 2354-60.
29. Goodwin and Rottman (1992) J. Biol. Chem. 267(23): 16330-4.
30. Green et al. (2003) J. Virol. 77(3): 2046-55.
31. Haigwood and Stamatatos (2003) AIDS 17 (Suppl 4): S67-71.
32. Hanke and McMichael (2000) Nat. Med. 6(9): 951-5.
33. Hanke et al. (1992) J Gen Virol. 73 (Pt 3): 653-60.
34. Hanke et al. (1998a) Vaccine 16(5): 439-45.
35. Hanke et al. (1998b) Vaccine 16(4): 426-35.
36. Hanke et al. (1998c) J. Gen. Virol. 79 (Pt 1): 83-90.
37. Hanke et al. (1999) J. Virol. 73(9): 7524-32.
38. Hanke et al. (2002a) Vaccine 20(15): 1995-8.
39. Hanke et al. (2002b) Vaccine 21(1-2): 108-14.
40. Hanke et al. (2003) J Gen Virol. 84(Pt 2): 361-8.
41. Harlow and Lane, Using Antibodies: A Laboratory Manual (1998) Cold Spring Harbor Laboratory Press, Cold Spring Harbor, N.Y.
42. Hel et al. (2001) J. Immunol. 167(12): 7180-91.
43. Hoffman et al. (1994) Vaccine 12(16): 1529-33.
44. Jin et al. (1999) J Exp Med. 189(6): 991-8.
45. Johnston and Tang (1994) Methods Cell Biol. 43 Pt A: 353-65.
46. Jung et al. (2002) Nature 418(6894): 144.
47. Kaul et al. (2001) J Clin Invest. 107(10): 1303-10.
48. Kent et al. (1998) J. Virol. 72(12): 10180-8.
49. Kotloff et al. (2000) Infect Immun. 68(3): 1034-9.
50. Kozak (1987) Nucleic Acids Res. 15(20): 812548.
51. Lifson et al. (2001) J. Virol. 75(21): 10187-99.
52. Lingnau, et al. (2002) Vaccine 20(29-30): 3498-508.
53. Lundstrom (2002) Technol Cancer Res Treat. 1(1): 83-8.
54. Matano et al. (1998) J. Virol. 72(1): 164-9.
55. Mayr et al. (1975) Infection 105: 6-14.
56. McConkey et al. (2003) Nat. Med. 9(6): 729-35.
57. McEwan and Gatherer (1998) Biotechniques 24(1): 131-6.
58. McMichael and Rowland-Jones (2001) Nature 410(6831): 980-7.
59. McSorley et al. (2002) J. Immunol. 169(7): 3914-9.
60. Meyer et al. (1991) J. Gen. Virol. 72 (Pt 5): 1031-8.
61. Morency et al. (1987) Biotechniques 5(5): 444-448.
62. Mowat et al. (2001) J. Immunol. 167(6): 3398-405.
63. Nakamura et al. (1996) Nucleic Acids Res. 24(1): 214-5.
64. Ogg et al. (1998) Science 279(5359): 2103-6.
65. Palmer et al. (1999) Arch Virol. 144(7): 1345-60.
66. Pantaleo, G. et al., Retroviral Immunology: Immune Response and Restoration (Infectious Disease) (2001) Humana Press, Totowa, N.J., pp. 1-31.
67. Pasetti et al. (1999) Clin Immunol. 92(1): 76-89.
68. Payne et al. (1995) Pharm Biotechnol. 6: 473-93.
69. Polo and Dubensky (1998) Nat. Biotechnol. 16(6): 517-8.
70. Remington, J.P. Remington's Pharmaceutical Sciences, 17th Edition, (1985) Mack Publishing Company, Easton, Pa.
71. Richardson, Baculovirus Expression Protocols: Methods in Molecular Biology, Volume 39 (1995), Humana Press, Totowa, N.J. 72. Robinson et al. (1993) Vaccine 11(9): 957-60.
73. Romero et al. (1989) Nature 341(6240): 323-6.
74. Rowland-Jones et al. (1998) J Clin Invest. 102(9): 1758-65.
75. Ruben et al. (1989) J. Virol. 63(1): 1-8.
76. Sambrook and Russell, Molecular Cloning: A Laboratory Manual, (1989) 2nd Edition, Cold Spring Harbor Laboratory Press, Cold Spring Harbor, N.Y.
77. Schellack et al. (2003) Proceedings of the 34th Annual Meeting of the German Society of Immunology, Berlin, September 24-27.
78. Schmitz et al. (1999) Science 283(5403): 857-60.
79. Schneider et al. (1998) Nat. Med. 4(4): 397-402.
80. Seed and Sheen (1988) Gene 67(2): 271-7.
81. Shata et al. (2000) Mol Med Today 6(2): 66-71.
82. Shiver et al. (2002) Nature 415(6869): 331-5.
83. Singh et al. (2002) J. Immunol. 168(1): 379-91.
84. Smerdou and Liljestrom (2000) Gene Ther. Regul. 1: 33-63.
85. Smith and Johnson (1988) Gene 67(1): 31-40.
86. Tacket et al. (1992) Vaccine 10(7): 443-6.
87. Tacket et al. (1997) Infect Immun. 65(2): 452-6.
88. Takahashi et al. (1988) Proc. Natl. Acad. Sci. USA. 85(9): 3105-9.
89. Veazey et al. (2003) J. Exp. Med. 198(10): 1551-62.
90. Voller, A. New Trends and Developments in Vaccines, (1978) University Park Press, Baltimore, Md.
91. Wang et al. (1998) Mol. Biotechnol. 10(2): 103-6.
92. Watanabe et al. (1994) Mol Reprod Dev. 38(3): 268-74.
93. Webster et al. (1994) Vaccine 12(16): 1495-8.
94. Wee et al. (2002) J. Gen. Virol. 83(Pt 1): 75-80.
95. Whittle et al. (1987) Protein Eng. 1(6): 499-505.
96. Williams et al. (1998) Nucleic Acids Res. 26(9): 2120-4.
97. Xiang et al. (1994) Virology 199(1): 132-40.

The invention is further described by the following numbered paragraphs:

1. An artificial fusion protein (AFP) comprising:

a) an HIV tat domain which lacks the nuclear localization signal, the integrin interaction domain and transactivation activity;

b) one or more HIV reverse transcriptase domains, each of which lacks polymerase activity; c) an HIV nef domain which can not be myristylated;

d) two CTL-rich domains from HIV gp41, wherein the first domain consists essentially of amino acids 699-742 of SEQ ID NO: 1 or the equivalent amino acids from gp41 of an HIV isolate or an HIV consensus sequence, and wherein the second domain consists essentially of amino acids 743-843 of SEQ ID NO: 1 or the equivalent amino acids from gp41 of an HIV isolate or an HIV consensus sequence; and

e) one or more human HIV CTL epitopes associated with long term non-progression to AIDS.

2. The AFP of Paragraph 1, wherein each of said HIV tat, reverse transcriptase, nef, and CTL-rich domains and each of said human HIV CTL epitopes are selected so that said AFP stimulates an immune response to a pre-determined HIV Clade.

3. The AFP of Paragraph 2, wherein said HIV Clade is selected from the group consisting of Clade A, A1, A2, B, C and D.

4. The AFP of Paragraph 3, wherein said HIV Clade is Clade A.

5. The AFP of Paragraph 1, wherein said the amino acid sequences for each of said HIV tat, reverse transcriptase, nef, and CTL-rich domains and each of said human HIV CTL epitopes are from an HIV consensus sequence for the same HIV Clade.

6. The AFP of Paragraph 5, wherein said HIV Clade is selected from the group consisting of Clade A, A1, A2, B, C and D.

7. The AFP of Paragraph 6, wherein said HIV Clade is Clade A.

8. The AFP of Paragraph 1, wherein said domains are present from N- to C-terminus in any order that does not recreate a naturally-occurring HIV protein.

9. The AFP of Paragraph 8, wherein said domains are joined with or without intervening sequences.

10. The AFP of Paragraph 1, wherein said domains are present from N- to C-terminus in order HIV tat domain, first HIV reverse transcriptase domain, HIV nef domain, second HIV reverse transcriptase domain, the first CTL-rich domain from HIV gp41, the second CTL-rich domain from HIV gp41 and the human HIV CTL epitope.

11. The AFP of Paragraph 10, wherein said domains are joined with or without intervening sequences.

12. The AFP of Paragraph 1, wherein said HIV tat domain comprises a sequence of amino acids from an HIV isolate or an HIV consensus sequence corresponding to amino acids 1-92 of SEQ ID NO: 1.

13. The AFP of Paragraph 12, wherein said HIV tat domain comprises amino acids 1-92 of SEQ ID NO: 1.

14. The AFP of Paragraph 1, which comprises two HIV reverse transcriptase domains.

15. The AFP of Paragraph 14, wherein one HIV reverse transcriptase domain comprises a sequence of amino acids from an HIV isolate or an HIV consensus sequence corresponding to amino acids 93-270 of SEQ ID NO: 1 and the second HIV reverse transcriptase domain comprises a sequence of amino acids from an HIV isolate or an HIV consensus sequence corresponding to amino acids 417-686 or 417-687 of SEQ ID NO: 1.

16. The AFP of Paragraph 15, wherein one HIV reverse transcriptase domain comprises amino acids 93-270 of SEQ ID NO: 1 and the other domain comprises amino acids 417-686 or 417-687 of SEQ ID NO: 1.

17. The AFP of Paragraph 1, wherein said HIV nef domain comprises a sequence of amino acids from an HIV isolate or an HIV consensus sequence corresponding to amino acids 273-416 of SEQ ID NO: 1.

18. The AFP of Paragraph 17, wherein said HIV nef domain comprises amino acids 273-416 of SEQ ID NO: 1.

19. The AFP of Paragraph 1, wherein the first CTL-rich domain from gp41 consists essentially of amino acids 699-742 of SEQ ID NO: 1, and wherein the second CTL-rich domain from gp41 consists essentially of amino acids 743-843 of SEQ ID NO: 1.

20. The AFP of Paragraph 1, wherein said one or more human HIV CTL epitopes associated with long term non-progression to AIDS has an amino acid sequence selected from the group consisting of TPGPGVRYPL (SEQ ID NO: 5), SPRTLNAWV (SEQ ID NO: 6), DTVLEDINL (SEQ ID NO: 4), ETAYFILKL (SEQ ID NO: 7), SLYNTVATL (SEQ ID NO: 8), AIFQSSMTK (SEQ ID NO: 9), YPLTFGWCF (SEQ ID NO: 10), ALKHRAYEL (SEQ ID NO: 11), LSPRTLNAW (SEQ ID NO: 12), VSFEPIPIHY (SEQ ID NO: 13), KIRLRPCGK (SEQ ID NO: 14), DLNMMLNIV (SEQ ID NO: 15), DRFWKTLRA (SEQ ID NO: 16), and ATPQDLNMML (SEQ ID NO: 17).

21. The AFP of Paragraph 1 comprising one human HIV CTL epitope associated with long term non-progression to AIDS.

22. The AFP of Paragraph 21, wherein said human HIV CTL epitope has an amino acid sequence selected from the group consisting of TPGPGVRYPL (SEQ ID NO: 5), SPRTLNAWV (SEQ ID NO: 6), DTVLEDINL (SEQ ID NO: 4), ETAYFILKL (SEQ ID NO: 7), SLYNTVATL (SEQ ID NO: 8), AIFQSSMTK (SEQ ID NO: 9), YPLTFGWCF (SEQ ID NO: 10), ALKHRAYEL (SEQ ID NO: 11), LSPRTLNAW (SEQ ID NO: 12), VSFEPIPIHY (SEQ ID NO: 13), KIRLRPCGK (SEQ ID NO: 14), DLNMMLNIV (SEQ ID NO: 15), DRFWKTLRA (SEQ ID NO: 16), and ATPQDLNMML (SEQ ID NO: 17).

23. The AFP of Paragraph 22, wherein said human HIV CTL epitope has the amino acid sequence DTVLEDINL (SEQ ID NO: 4).

24. The AFP of Paragraph 1, comprising amino acids 1-843 of SEQ ID NO: 1.

25. The AFP of any one of Paragraphs 1-24, which comprises one or more non-human CTL domains for monitoring immune responses to said AFP in a laboratory mammal.

26. The AFP of Paragraph 25, wherein said one or more additional domains is selected from the group consisting of the SIV (at CTL epitope, the pb9 epitope, the P18-I10 epitope and the SIV gag p27 epitope.

27. The AFP of Paragraph 26, wherein said additional domains are the SIV tat CTL epitope and the pb9 epitope.

28. The AFP of Paragraph 25, which comprises a marker domain.

29. The AFP of Paragraph 28, wherein said marker domain encodes an epitope for a monoclonal antibody selected from the group consisting of Pk, Flag, HA, myc, GST or H is epitopes.

30. The AFP of Paragraph 29, wherein said marker domain encodes the Pk epitope.

31. The AFP of Paragraph 1, comprising amino acids 1-871 of SEQ ID NO: 1.

32. An isolated nucleic acid having a nucleotide sequence encoding the AFP of any one of Paragraphs 1-24.

33. An isolated nucleic acid having a nucleotide sequence encoding the AFP of Paragraph 25.

34. An isolated nucleic acid having a nucleotide sequence encoding the AFP of Paragraph 35.

35. An isolated nucleic acid having a nucleotide sequence encoding the AFP of Paragraph 31.

36. An isolated nucleic acid, wherein said nucleic acid has a nucleotide sequence comprising SEQ ID NO: 2.

37. An expression vector comprising a nucleic acid having a nucleotide sequence encoding the AFP of any one of Paragraphs 1-24 operably linked to at least one nucleic acid control sequence.

38. An expression vector comprising a nucleic acid having a nucleotide sequence encoding the AFP of Paragraph 25 operably linked to at least one nucleic acid control sequence.

39. An expression vector comprising a nucleic acid having a nucleotide sequence encoding the AFP of Paragraph 28 operably linked to at least one nucleic acid control sequence.

40. An expression vector comprising a nucleic acid having a nucleotide sequence encoding the AFP of Paragraph 31 operably linked to at least one nucleic acid control sequence.

41. The expression vector of Paragraph 40, wherein said vector is a plasmid vector, a viral vector, an insect vector, a yeast vector or a bacterial vector.

42. The expression vector of Paragraph 41, wherein said plasmid vector is pTH or pTHr.

43. The expression vector of Paragraph 41, wherein said viral vector is an alphavirus replicon vector, an adeno-associated virus vector, an adenovirus vector, a retrovirus vector or a vaccinia virus vector.

44. The expression vector of Paragraph 43, wherein said vector is a vaccinia virus vector.

45. The expression vector of Paragraph 44, wherein said vaccinia virus is modified vaccinia Ankara (MVA).

46. The expression vector of Paragraph 41, wherein said bacterial vector is a live, attenuated Salmonella or a Shigella vector.

47. The expression vector of Paragraph 40, wherein said nucleic acid control sequence is a cytomegalovirus (CMV) immediate early promoter.

48. The expression vector of any one of Paragraphs 40-47, wherein the codons encoding said AFP are those of highly expressed genes for a target organism or host cell in which said AFP is to be expressed.

49. The expression vector of Paragraph 48, wherein the target organism or host cell is a human.

50. The expression vector of Paragraph 42, wherein said expression vector and nucleic acid together is pTHr.RENTA.

51. The expression vector of Paragraph 45, wherein said expression vector and nucleic acid together is MVA.RENTA.

52. A host cell comprising the expression vector of Paragraph 37.

53. A host cell comprising the expression vector of Paragraph 39.

54. A host cell comprising the expression vector of Paragraph 40.

55. A host cell comprising the expression vector of Paragraph 41.

56. A host cell comprising the expression vector of Paragraph 48.

57. A host cell comprising the expression vector of Paragraph 50.

58. A host cell comprising the expression vector of Paragraph 51.

59. A method of preparing an AFP, which comprises (a) culturing the host cell of Paragraph 52 for a time and under conditions to express said AFP; and (b) recovering said AFP.

60. A method of preparing an AFP, which comprises (a) culturing the host cell of any one of Paragraphs 54, 55 or 57 for a time and under conditions to express said AFP; and (b) recovering said AFP.

61. A method for introducing into and expressing an AFP in an animal, which comprises delivering an expression vector of Paragraph 37 into said animal and thereby obtaining expression of the AFP in said animal.

62. A method for introducing into and expressing an AFP in an animal, which comprises delivering an expression vector of Paragraph 38 into said animal and thereby obtaining expression of the AFP in said animal.

63. A method for introducing into and expressing an AFP in an animal, which comprises delivering an expression vector of Paragraph 39 into said animal and thereby obtaining expression of the AFP in said animal.

64. A method for introducing into and expressing an AFP in an animal, which comprises delivering an expression vector of any one of Paragraphs 40-47, 50 or 51 into said animal and thereby obtaining expression of the AFP in said animal.

65. A method for expressing an AFP in animal cells, which comprises (a) introducing an expression vector of Paragraph 37 into said animal cells; and (b) culturing those cells under conditions sufficient to express said AFP.

66. A method for expressing an AFP in animal cells, which comprises (a) introducing an expression vector of Paragraph 38 into said animal cells; and (b) culturing those cells under conditions sufficient to express said AFP.

67. A method for expressing an AFP in animal cells, which comprises (a) introducing an expression vector of Paragraph 39 into said animal cells; and (b) culturing those cells under conditions sufficient to express said AFP.

68. A method for expressing an AFP in animal cells, which comprises (a) introducing an expression vector of Paragraph 40-47, 50 or 51 into said animal cells; and (b) culturing those cells under conditions sufficient to express said AFP.

69. A method for inducing an immune response in an animal, which comprises delivering an expression vector of Paragraph 37 into said animal, wherein said AFP is expressed at a level sufficient to stimulate an immune response to AFP.

70. A method for inducing an immune response in an animal, which comprises delivering an expression vector of Paragraph 38 into said animal, wherein said AFP is expressed at a level sufficient to stimulate an immune response to AFP.

71. A method for inducing an immune response in an animal, which comprises delivering an expression vector of Paragraph 39 into said animal, wherein said AFP is expressed at a level sufficient to stimulate an immune response to AFP.

72. A method for inducing an immune response in an animal, which comprises delivering an expression vector of any one of Paragraphs 40-47, 50 or 51 into said animal, wherein said AFP is expressed at a level sufficient to stimulate an immune response to AFP.

73. A method for inducing an immune response in an animal, which comprises delivering an AFP of any one of Paragraphs 1-24 into said animal in an amount sufficient to stimulate an immune response to AFP.

74. A method for inducing an immune response in an animal, which comprises delivering an AFP of Paragraph 25 into said animal in an amount sufficient to stimulate an immune response to AFP.

75. A method for inducing an immune response in an animal, which comprises delivering an AFP of Paragraph 28 into said animal in an amount sufficient to stimulate an immune response to AFP.

76. A method for inducing an immune response in an animal, which comprises delivering an AFP of Paragraph 31 into said animal in an amount sufficient to stimulate an immune response to AFP.

77. A method of stimulating an immune response against HIV in a human subject, which comprises administering an immunogen one or more times to a subject, wherein said immunogen is selected from the group consisting of (i) an AFP of any one of Paragraphs 1-24 or 31, (ii) a nucleic encoding said AFP, and (iii) an expression vector encoding said AFP; and wherein said AFP is administered in an amount or expressed at a level sufficient to stimulate an HIV-specific CTL immune response in said subject.

78. The method of Paragraph 77, wherein said subject receives at least two administrations of said immunogen at intervals of at least two weeks or at least four weeks.

79. The method of Paragraph 78, wherein another HIV immunogen is administered at the same time or at different times as part of an overall immunization regime.

80. A method of stimulating an immune response against HIV in a human subject, which comprises administering to said subject at least one priming dose of an HIV immunogen and at least one boosting dose of an HIV immunogen, wherein said immunogen in each dose can be the same or different, provided that at least one of said immunogens is an AFP of any one of Paragraphs 1-24 or 31 or is a nucleic acid or an expression vector encoding said AFP, wherein said immunogens are administered in an amount or expressed at a level sufficient to stimulate an HIV-specific T-cell immune response in said subject.

81. The method of Paragraph 80, wherein the interval between each dose is at least two weeks or at least four weeks.

82. The method of Paragraph 80, wherein pTHr.RENTA is administered one or more times as a priming dose.

83. The method of Paragraph 80, wherein MVA.RENTA is administered one or more times as a boosting dose.

84. The method of Paragraph 82, wherein MVA.RENTA is administered one or more times as a boosting dose.

85. The method of Paragraph 80, wherein the HIV immunogen for at least one priming dose is pTHr.HIVA and the HIV immunogen for at least one boosting dose is MVA.RENTA.

86. The method of Paragraph 80, wherein the HIV immunogen for at least one priming dose is pTHr.HIVA and the HIV immunogen for at least one boosting dose is a mixture of MVA.RENTA and MVA.HIVA.

87. The method of Paragraph 80, wherein the HIV immunogen for at least one priming dose is pTHr.RENTA and the HIV immunogen for at least one boosting dose is MVA.HIVA.

88. The method of Paragraph 80, wherein the HIV immunogen for at least one priming dose is pTHr.RENTA and the HIV immunogen for at least one boosting dose is a mixture of MVA.RENTA and MVA.HIVA.

89. The method of Paragraph 80, wherein the HIV immunogen for at least one priming dose is a mixture of pTHr.HIVA and pTHr.RENTA and the HIV immunogen for at least one boosting dose is MVA.HIVA.

90. The method of Paragraph 80, wherein the HIV immunogen for at least one priming dose is a mixture of pTHr.HIVA and pTHr.RENTA and the HIV immunogen for at least one boosting dose is MVA.RENTA.

91. The method of Paragraph 80, wherein the HIV immunogen for at least one priming dose is a mixture of pTHr.HIVA and pTHr.RENTA and the HIV immunogen for at least one boosting dose is a mixture of MVA.RENTA and MVA.HIVA.

92. The method of Paragraph 80, which comprises administering two priming doses and administering two boosting doses, wherein the immunogen used for the priming doses is a plasmid vector and the immunogen used for the boosting doses is a viral vector.

93. The method of Paragraph 82, wherein said viral vector is an MVA vector.

94. The method of Paragraph 92, wherein each of said priming doses is a mixture of pTHr.HIVA and pTHr.RENTA and each of said boosting doses is a mixture of MVA.RENTA and MVA.HIVA.

95. An immunogenic composition comprising an AFP of any one of Paragraphs 1-24 or 31, a nucleic acid encoding said AFP or an expression vector encoding said AFP; and a pharmaceutically acceptable carrier.

96. An immunogenic composition comprising an AFP of Paragraph 25 or a nucleic acid encoding said AFP or an expression vector encoding said AFP; and a pharmaceutically acceptable carrier.

97. An immunogenic composition comprising an AFP of Paragraph 28, a nucleic acid encoding said AFP or an expression vector encoding said AFP; and a pharmaceutically acceptable carrier.

98. An immunogenic composition comprising an expression vector of any one of Paragraphs 40-47, 50 or 51; and a pharmaceutically acceptable carrier.

99. The composition of Paragraph 95, which further comprises an adjuvant.

100. The composition of Paragraph 96, which further comprises an adjuvant.

101. The composition of Paragraph 97, which further comprises an adjuvant.

102. The composition of Paragraph 98, which further comprises in adjuvant.

103. The composition of Paragraph 102, wherein said adjuvant is selected from the group consisting of mineral salts, polynucleotides, polyarginines, ISCOMs, saponins, monophosphoryl lipid A, imiquimod, CCR-5 inhibitors, toxins, polyphosphazenes, cytokines, immunoregulatory proteins, immunostimulatory fusion proteins, co-stimulatory molecules, and combinations thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An artificial fusion protein (AFP) comprising:
a) an HIV tat domain which lacks the nuclear localization signal, the integrin interaction domain and transactivation activity;
b) one or more HIV reverse transcriptase domains, each of which lacks polymerase activity;
c) an HIV nef domain which can not be myristylated;
d) two CTL-rich domains from HIV gp41, wherein the first domain consists essentially of amino acids 699-742 of SEQ ID NO: 1 or the equivalent amino acids from gp41 of an HIV isolate or an HIV consensus sequence, and wherein the second domain consists essentially of amino acids 743-843 of SEQ ID NO: 1 or the equivalent amino acids from gp41 of an HIV isolate or an HIV consensus sequence; and
e) one or more human HIV CTL epitopes associated with long term non-progression to AIDS.

2. The AFP of claim 1, wherein each of said HIV tat, reverse transcriptase, nef, and CTL-rich domains and each of said human HIV CTL epitopes are selected so that said AFP stimulates an immune response to a pre-determined HIV Clade.

3. The AFP of claim 2, wherein said HIV Clade is selected from the group consisting of Clade A, A1, A2, B, C and D.

4. The AFP of claim 3, wherein said HIV Clade is Clade A.

5. The AFP of claim 1, wherein said the amino acid sequences for each of said HIV tat, reverse transcriptase, nef, and CTL-rich domains and each of said human HIV CTL epitopes are from an HIV consensus sequence for the same HIV Clade.

6. The AFP of claim 5, wherein said HIV Clade is selected from the group consisting of Clade A, A1, A2, B, C and D.

7. The AFP of claim 6, wherein said HIV Clade is Clade A.

8. The AFP of claim 1, wherein said domains are present from N— to C-terminus in any order that does not recreate a naturally-occurring HIV protein.

9. The AFP of claim 8, wherein said domains are joined with or without intervening sequences.

10. The AFP of claim 1, wherein said domains are present from N— to C-terminus in order HIV tat domain, first HIV reverse transcriptase domain, HIV nef domain, second HIV reverse transcriptase domain, the first CTL-rich domain from HIV gp41, the second CTL-rich domain from HIV gp41 and the human HIV CTL epitope.

11. The AFP of claim 10, wherein said domains are joined with or without intervening sequences.

12. The AFP of claim 1, wherein said HIV tat domain comprises a sequence of amino acids from an HIV isolate or an HIV consensus sequence corresponding to amino acids 1-92 of SEQ ID NO: 1.

13. The AFP of claim 12, wherein said HIV tat domain comprises amino acids 1-92 of SEQ ID NO: 1.

14. The AFP of claim 1, which comprises two HIV reverse transcriptase domains.

15. The AFP of claim 14, wherein one HIV reverse transcriptase domain comprises a sequence of amino acids from an HIV isolate or an HIV consensus sequence corresponding to amino acids 93-270 of SEQ ID NO: 1 and the second HIV reverse transcriptase domain comprises a sequence of amino acids from an HIV isolate or an HIV consensus sequence corresponding to amino acids 417-686 or 417-687 of SEQ ID NO: 1.

16. The AFP of claim 15, wherein one HIV reverse transcriptase domain comprises amino acids 93-270 of SEQ ID NO: 1 and the other domain comprises amino acids 417-686 or 417-687 of SEQ ID NO: 1.

17. The AFP of claim 1, wherein said HIV nef domain comprises a sequence of amino acids from an HIV isolate or an HIV consensus sequence corresponding to amino acids 273-416 of SEQ ID NO: 1.

18. The AFP of claim 17, wherein said HIV nef domain comprises amino acids 273-416 of SEQ ID NO: 1.

19. The AFP of claim 1, wherein the first CTL-rich domain from gp41 consists essentially of amino acids 699-742 of SEQ ID NO: 1, and wherein the second CTL-rich domain from gp41 consists essentially of amino acids 743-843 of SEQ ID NO: 1.

20. The AFP of claim 1, wherein said one or more human HIV CTL epitopes associated with long term non-progression to AIDS has an amino acid sequence selected from the group consisting of TPGPGVRYPL (SEQ ID NO: 5), SPRTLNAWV (SEQ ID NO: 6), DTVLEDINL (SEQ ID NO: 4), ETAYFILKL (SEQ ID NO: 7), SLYNTVATL (SEQ ID NO: 8), AIFQSSMTK (SEQ ID NO: 9), YPLTFGWCF (SEQ ID NO: 10), ALKHRAYEL (SEQ ID NO: 11), LSPRTLNAW (SEQ ID NO: 12), VSFEPIPIHY (SEQ ID NO: 13), KIRLRPCGK (SEQ ID NO: 14), DLNMMLNIV (SEQ ID NO: 15), DRFWKTLRA (SEQ ID NO: 16), and ATPQDLNMML (SEQ ID NO: 17).

21. The AFP of claim 1 comprising one human HIV CTL epitope associated with long term non-progression to AIDS.

22. The AFP of claim 21, wherein said human HIV CTL epitope has an amino acid sequence selected from the group consisting of TPGPGVRYPL (SEQ ID NO: 5), SPRTLNAWV (SEQ ID NO: 6), DTVLEDINL (SEQ ID NO: 4), ETAYFILKL (SEQ ID NO: 7), SLYNTVATL (SEQ ID NO: 8), AIFQSSMTK (SEQ ID NO: 9), YPLTFGWCF (SEQ ID NO: 10), ALKHRAYEL (SEQ ID NO: 11), LSPRTLNAW (SEQ ID NO: 12), VSFEPIPIHY (SEQ ID NO: 13), KIRLRPCGK (SEQ ID NO: 14), DLNMMLNIV (SEQ ID NO: 15), DRFWKTLRA (SEQ ID NO: 16), and ATPQDLNMML (SEQ ID NO: 17).

23. The AFP of claim 22, wherein said human HIV CTL epitope has the amino acid sequence DTVLEDINL (SEQ ID NO: 4).

24. The AFP of claim 1, comprising amino acids 1-843 of SEQ ID NO: 1.

25. The AFP of claim 1, which comprises one or more non-human CTL domains for monitoring immune responses to said AFP in a laboratory mammal.

26. The AFP of claim 25, wherein said one or more additional domains is selected from the group consisting of the SIV (at CTL epitope, the pb9 epitope, the P18-h10 epitope and the SIV gag p27 epitope.

27. The AFP of claim 26, wherein said additional domains are the SIV tat CTL epitope and the pb9 epitope.

28. The AFP of claim 25, which comprises a marker domain.

29. The AFP of claim 28, wherein said marker domain encodes an epitope for a monoclonal antibody selected from the group consisting of Pk, Flag, HA, myc, GST or H is epitopes.

30. The AFP of claim 29, wherein said marker domain encodes the Pk epitope.

31. The AFP of claim 1, comprising amino acids 1-871 of SEQ ID NO: 1.
